(19) United States
(12) Patent Application Publication
(10) Pub. No.: US 2026/0007747 A1
(43) Pub. Date: Jan. 8, 2026

(54) Engineered PD-L1-Targeting Gamma Delta T Cell Receptors

(71) Applicant: Vivasor, Inc., San Diego, CA (US)

(72) Inventors: Runqiang Chen, San Diego, CA (US); Changyou Lin, San Diego, CA (US); Xiaomei Yuan, San Diego, CA (US); Hong Pei, San Diego, CA (US); Zhao Chen, San Diego, CA (US); Jiqing Xu, San Diego, CA (US); Henry Hongjun Ji, Rancho Santa Fe, CA (US)

(73) Assignee: Vivasor, Inc., San Diego, CA (US)

(21) Appl. No.: 18/727,012

(22) PCT Filed: Jan. 6, 2023
(86) PCT No.: PCT/US2023/010354

§ 371 Date: Jul. 5, 2024

Related U.S. Application Data
(60) Provisional application No. 63/297,619, filed on Jan. 7, 2022.

(51) Int. Cl. A61K 40/32; A61K 40/11; A61K 40/42; A61P 35/00; C07K 14/725; C07K 16/28

(52) U.S. Cl. CPC A61K 40/32; A61K 40/11; A61K 40/4211; A61K 40/4224; A61P 35/00; C07K 14/7051; C07K 16/2803; C07K 16/2827; C07K 2317/622

(57) ABSTRACT

The present disclosure provides engineered chimeric T cell receptors and subunit polypeptides that include a single chain antibody (ScFv) that binds a target antigen fused to at least a portion of a constant region of a TCRγ chain or at least a portion of a constant region of a TCRδ chain. Also provided are nucleic acid molecules that encode the engineered T cell receptors and subunit polypeptides, transgenic cells expressing the engineered T cell receptors and polypeptides, and methods of using the transgenic cells for the treatment of cancer.

### CROSS-REFERENCE TO RELATED APPLICATIONS

This application claims priority to U.S. Provisional Application No. 63/297,619, filed Jan. 7, 2022, the disclosure of which is hereby incorporated by reference in its entirety.

### TECHNICAL FIELD

The present disclosure provides engineered T cell receptors and subunit polypeptides that include a single chain antibody (ScFv) that binds a target antigen fused to at least a portion of a constant region of a TCRγ chain or at least a portion of a constant region of a TCRδ chain. Also included are nucleic acid molecules encoding the engineered TCR polypeptides, host cells that express the engineered polypeptides and TCRs, and methods of use in treating cancer.

### BACKGROUND

T cells are able to attack and destroy diseased or malignant cells as observed in viral infections and rare spontaneous remissions of cancer. T cells are easily tolerized to self or tumor antigens however, such that tumors are able to avoid or escape immune surveillance. Strategies have been developed to provide target specificity and affinity to a patient's T cells by engineering the T cells to express recombinant receptors such as chimeric antigen receptors (CARs). CARs provide antibody-mediated cancer cell marker recognition and intracellular signaling capability to direct the host T cells to kill malignant cells based on their expression of an antigen recognized by the CAR.

Adoptive immunotherapy by infusion of T cells engineered with chimeric antigen receptors (CAR-T cells) can provide a potentially highly specific and effective treatment of cancer (sec. e.g., Sadelain et al. (2013) Cancer Discovery 3:388-398). Treatment with CAR-T cells however can result in cytokine release syndrome (CRS) in the patient which can be severe and potentially life-threatening (Frey & Porter (2019) Biol Blood Marrow Transplant 25:e123-e127). Cytokines found to be elevated in CAR-T patients exhibiting CRS include interferon gamma, the soluble αIL-2 receptor, IL-6, and IL-10. Recent studies by Sacheva et al. (2019, J. Biol. Chem. 294:5430-37) indicate that eliminating GM-CSF expression by engineered T cells may reduce the secretion of other cytokines implicated in CRS.

Tumor cells can escape destruction by the immune system by engaging inhibitory immune checkpoint pathways (Pardoll (2012) Nature Reviews Cancer Vol. 12:252-264; Darvin et al. (2018) Exp Mol Med 50:1-11). Inhibitory immune checkpoint pathways, such as those mediated by interactions of immune checkpoint proteins PD-1. PD-L1, CTLA-4. LAG-3, TIM3, and TIGIT counteract immune system activation to prevent autoimmune responses. Tumor cells can take advantage of these inhibitory pathways by expressing immune checkpoint proteins that interact with their counterparts on T cells, resulting in de-activation of the T cells and shutting down of the anti-tumor immune response. Immune checkpoint proteins inhibit the activation or function of T-cells to regulate the intensity and duration of immune responses and maintain self-tolerance. Numerous immune checkpoint proteins are known, such as PD-1 (Programmed Death 1) with its ligands PD-L1 and PD-L2, CTLA-4 (Cytotoxic T-Lymphocyte-Associated protein 4) and its ligands CD80 and CD86; TIM-3 (T-cell Immunoglobulin domain and Mucin domain 3), LAG-3 (Lymphocyte Activation Gene-3), TIGIT (T cell immunoreceptor with Ig and ITIM domains), BTLA (CD272 or B and T Lymphocyte Attenuator), and VISTA (V-domain immunoglobulin suppressor of T-cell activation) (Pardoll (2012) Nature Reviews Cancer 12:252-264; Borcherding et al. (2018) J Mol Biol 430:2014-2029).

### SUMMARY

The disclosure provides engineered single chain antibody T Cell Receptors (scFv-TCRs) comprising an engineered T cell receptor (TCR) subunit that includes an scFv that binds a target antigen. In various embodiments disclosed herein the scFv-TCRs are based on the structure of gamma delta T cell receptors (TCRγδs). In particular embodiments, recombinant receptors are described in which an scFv (for example an scFv that can specifically bind a tumor associated antigen or checkpoint protein) is linked to a portion of the TCRγ subunit, for example, linked to at least the transmembrane domain and intracellular domain of the TCRγ subunit or polypeptide sequences homologous thereto, where T cells expressing the recombinant scFv-TCRγ receptors can demonstrate rapid tumor-dependent expansion and potent anti-tumor activity. Particular illustrative embodiments provided herein include anti-PD-L1 scFv-TCRγ receptors and anti-CD19 scFv-TCRγ receptors.

In some aspects the disclosure provides host cells that include nucleic acid constructs for expressing the novel engineered TCR subunit receptors, such as nucleic acid molecules that encode an engineered polypeptide having an scFv linked to sequences derived from the TCRγ chain. Also included are host cells that include the nucleic acid constructs that encode the scFv-TCRγ polypeptides. The host cells can be, for example, T cells, and in various embodiments are T cells that do not express an alpha beta T cell receptor (TCRαβ). In various embodiments the T cells that do not express TCRαβ receptors can express the engineered scFv-TCRγ polypeptide in the absence of expression of a TCRδ chain or an engineered receptor polypeptide based on a TCRδ chain. As demonstrated in the Examples, engineered T cells expressing a single TCR polypeptide, for example, an engineered scFv-TCRγ polypeptide that includes an extracellular scFv as a targeting domain and transmembrane and intracellular domain sequences derived from the TCRγ chain, surprisingly are able to expand on target cells, exhibit target-specific activation, and kill target tumor cells in vitro and in vivo.

For example, engineered TCRγδs and engineered TCRγ and TCRδ subunits are provided herein. In any of the embodiments disclosed herein, the host cells, methods, and nucleic acid molecules may further include the following features, which may be combined with one another in any combinations unless clearly mutually exclusive:

The embodiments provided herein encompass the following:

A first embodiment is a host cell, or population of host cells, comprising an exogenous nucleic acid sequence encoding a chimeric single chain antibody TCR gamma subunit (scFv-TCRγ), wherein the scFv-TCRγ subunit comprises, from the N-terminus to the C-terminus:
an scFv that binds a target antigen; and either (1) a combined TCRγ connecting peptide, TCRγ transmembrane domain, and TCRγ intracellular domain sequence having at least 95% identity to SEQ ID NO:17; or (2) a TCRγ connecting peptide having at least 95% identity to SEQ ID NO:9,
a TCRγ transmembrane domain having at least 95% identity to SEQ ID NO:10, and
a TCRγ intracellular domain having at least 95% identity to SEQ ID NO:11; in which the host cell or population of host cells does not include an exogenous nucleic acid sequence encoding a TCR delta (TCRδ) subunit or an engineered polypeptide that includes an amino acid sequence having at least 95% identity to the connecting peptide of the TCRδ subunit (e.g., SEQ ID NO:14) and/or includes an amino acid sequence having at least 95% identity to the connecting peptide of the TCRδ subunit (e.g., SEQ ID NO:15). In various embodiments host cells do not express a native TCRδ subunit and do not express an engineered TCRδ subunit, i.e., a polypeptide having either or both of a connecting peptide or transmembrane domain of a TCRδ subunit, or amino acid sequences having at least 95% identity to either or both of a TCRδ connecting peptide (e.g., SEQ ID NO:14) or TCRδ transmembrane domain (e.g., SEQ ID NO:15).

In any of the above embodiments or those disclosed below that include a cell or cells that include a construct encoding an scFv-TCRγ polypeptide, the TCRγ connecting peptide may comprise, consist essentially of, or consist of a sequence having at least 95% identity to SEQ ID NO:9, the TCRγ transmembrane domain may comprise, consist essentially of, or consist of a sequence having at least 95% identity to SEQ ID NO:10, and/or the TCRγ intracellular domain may comprise, consist essentially of, or consist of a sequence having at least 95% identity to SEQ ID NO:11. In any of the above embodiments or those disclosed below that include a cell or cells that include a construct encoding an scFv-TCRγ polypeptide, the TCRγ connecting peptide may comprise, consist essentially of, or consist of SEQ ID NO:9, the TCRγ transmembrane domain may comprise, consist essentially of, or consist of SEQ ID NO:10, and/or the TCRγ intracellular domain may comprise, consist essentially of, or consist of a sequence having at least 95% identity to SEQ ID NO:11. In any of the above embodiments or those disclosed below that include a cell or cells including a construct encoding an scFv-TCRγ polypeptide, the scFv-TCRγ subunit can comprise, consist essentially of, or consist of a sequence having at least 95% identity to SEQ ID NO:17. In any of the above embodiments or those disclosed below that include a cell or cells that include a construct encoding an scFv-TCRγ polypeptide, the scFv-TCRγ subunit can comprise, consist essentially of, or consist of SEQ ID NO:17.

The target antigen can be any tumor associated antigen or can be a checkpoint inhibitor protein. In various embodiments the target antigen may be any of PD-L1, B7H3, BCMA, CD19, CD20, CD22, CD38, CD123, Claudin 18.2, EGFRVIII, GPC3, mesothelin, MUC1, or PSMA.

In some embodiments a host cell, or population of host cells, comprises an exogenous nucleic acid sequence encoding a chimeric single chain antibody TCR gamma subunit (scFv-TCRγ), in which the scFv-TCRγ subunit comprises an scFv that specifically binds PD-L1.

In some embodiment a host cell, or population of host cells, include an scFv-TCRγ subunit comprising an scFv having a heavy chain variable region having at least 95% identity to SEQ ID NO:1 and a light chain variable region having at least 95% identity to SEQ ID NO:2. The heavy chain variable region can be N-terminal to the light chain variable region or alternatively, the light chain variable region can be N-terminal to the heavy chain variable region. In some embodiments, the host cell, or population of host cells, comprises an exogenous nucleic acid sequence encoding a chimeric single chain antibody TCR gamma subunit (scFv-TCRγ), wherein the scFv-TCRγ subunit comprises a combined scFv, TCRγ connecting peptide, TCRγ transmembrane domain, and TCRγ intracellular domain sequence having at least 95% identity to SEQ ID NO:25. In some embodiments, the host cell or cells provided herein can include a nucleic acid construct encoding a PD-L1 targeting scFv-TCRγ, where the scFv comprises a heavy chain variable region comprising a heavy chain complementarity determining region (HCDR1) comprising SEQ ID NO:46, an HCDR2 comprising SEQ ID NO:47, and an HCDR3 comprising SEQ ID NO:48, and a light chain variable region comprising a light chain complementarity determining region (LCDR1) comprising SEQ ID NO:49, an LCDR2 comprising SEQ ID NO 50, and an LCDR3 comprising SEQ ID NO:51.

In some embodiments, which may be combined with any of the embodiments of a PD-L1 targeting scFv-TCRγ above, the host cell or cells can include a nucleic acid construct encoding a PD-L1 targeting scFv-TCRγ, where the scFv comprises a heavy chain variable region having at least 95% identity to SEQ ID NO:1 and a light chain variable region having at least 95% identity to SEQ ID NO:2, optionally wherein the heavy chain variable region comprises the sequence of SEQ ID NO:1 and the light chain variable region comprises the sequence of SEQ ID NO:2. In some embodiments, the host cell or population of host cells of any one of the preceding embodiments can include an exogenous nucleic acid sequence encoding an scFv-TCRγ where the scFv comprises an amino acid sequence having at least 95% identity to SEQ ID NO:4, optionally wherein the scFv comprises the sequence of SEQ ID NO:4.

In various embodiments of the host cell or population of host cells described herein, the target antigen bound by the scFv of the scFv-TCRγ is a tumor associated antigen.

In some embodiments of the host cell or population of host cells described herein, the target antigen bound by the scFv of the scFv-TCRγ is CD19. In some embodiments, a host cell or population of host cells that includes a nucleic acid sequence encoding an scFv-TCRγ as set forth in Embodiment 1, where the scFv-TCRγ subunit comprises a combined scFv, TCRγ connecting peptide, TCRγ transmembrane domain, and TCRγ intracellular domain sequence having at least 95% identity to SEQ ID NO:62. In some embodiments, the host cell or cells provided herein can include a nucleic acid construct encoding a CD19 targeting scFv-TCRγ, where the scFv comprises a heavy chain variable region having at least 95% identity to SEQ ID NO:58 and a light chain variable region having at least 95% identity to SEQ ID NO:59, optionally wherein the heavy chain variable region comprises the sequence of SEQ ID NO:58 and the light chain variable region comprises the sequence of SEQ ID NO:59. In some embodiments, the host cell or population of host cells of any one of the preceding embodiments can include an exogenous nucleic acid sequence encoding an scFv-TCRγ where the scFv comprises an amino acid sequence having at least 95% identity to SEQ ID NO:60, optionally wherein the scFv comprises the sequence of SEQ ID NO:60.

In any of the foregoing embodiments, the encoded scFv-TCRγ subunit can further comprise a signal peptide (signal sequence) at the N-terminus of the scFv-TCRγ subunit. Numerous signal peptides are known in the art, some of which are disclosed herein (e.g., SEQ ID NO:8, SEQ ID NO:13, SEQ ID NO:22, SEQ ID NO:23, SEQ ID NO:55, SEQ ID NO:56, SEQ ID NO:57).

In various embodiments a population of host cells according to any of the above-disclosed embodiments, wherein the exogenous nucleic acid sequences encoding the scFv-TCRγ polypeptide are inserted into a TCRα or TCRβ gene, where the TCRα or TCRβ subunit gene is inactivated.

In any of the embodiments the host cell or population of host cells disclosed herein, the host cell(s) can comprise T lymphocytes, natural killer (NK) cells, macrophages, dendritic cells, mast cells, eosinophils, B lymphocytes, or monocytes. In some embodiments, the host cell(s) comprise natural killer (NK) cells. In some embodiments, the host cell(s) comprise primary NK cells, which may optionally be derived from placental or cord blood. In some embodiments, the host cell(s) comprise NK cells derived from embryonic stem cells (ESCs) or induced pluripotent stem cells (iPSCs).

In any of the embodiments of a host cell or population of host cells provided herein, the host cell(s) may comprise T lymphocytes, for example, the a host cell or population of host cells may comprise primary human T cells.

Further embodiments include a population of host cells comprising a host cell of any of the previous embodiments, wherein at least 10%, at least 20%, at least 30%, at least 40%, at least 50%, at least 55%, at least 60%, at least 65%, at least 70%, at least 75%, or at least 80%, of the population of host cells expresses the chimeric scFv-TCRγ. In various embodiments the population is transfected or transduced with a construct encoding the chimeric scFv-TCRγ. In various embodiments at least 70%, at least 80%, at least 85%, at least 90%, or at least 95% of the host cell population as disclosed herein in any of the previous embodiments or description that expresses the chimeric scFv-TCRγ does not express an alpha beta T cell receptor (TCRαβ).

Additional embodiments are the host cell or population of host cells of any one of the previously disclosed embodiments for use in therapy.

Additional embodiments are the host cell or population of host cells of any one of the previously disclosed embodiments for the manufacture of a medicament for treating cancer.

Also provided herein is a pharmaceutical composition for use in treating a subject with cancer, wherein the treating comprises administering a composition comprising a host cell or a population of host cells according to of the above provided embodiments to a patient having cancer. In various embodiments, wherein said administering is performed more than once.

Embodiments include a pharmaceutical composition comprising a host cell or a population of host cells according to any of the above-described embodiments or described herein. The host cell(s) can be T cells, and can be primary T cells, such as primary human T cells. The pharmaceutical composition can include a buffer, e.g., a physiologically compatible buffer for maintaining live cells, and may include, for example, PBS, HBSS, Tyrode's solution, or Ringer's solution, or modified versions thereof. The composition can optionally include a cryoprotectant such as glycerol or DMSO, and the pharmaceutical composition can optionally be provided as a frozen composition.

Additional embodiments are the use, host cell, or population of host cells for use, or pharmaceutical composition of any one of the previously disclosed embodiments, where the medicament, host cell, population of host cells, or pharmaceutical composition is for administration by injection or infusion. In various embodiments, wherein said administering is performed more than once.

Additional embodiments are the use, host cell, or population of host cells for use, or pharmaceutical composition of any one of the embodiments above or described herein, wherein 10⁴ to 10¹¹ cells are administered to the subject.

In any of the afore-mentioned embodiments on the use of host cells, or on the host cell, or population of host cells for use, or on pharmaceutical compositions or their use, the host cells or populations of host cells may be allogeneic with respect to the subject.

Further provided are methods for treating a subject with cancer, where the treating comprises administering a composition comprising a host cell or a population of host cells according to any provided herein to a subject with cancer. Administration of cells can be by injection or infusion, for example, where from 10⁴ to 10¹¹ cells can be administered to the subject in a single dosing. A subject can receive more than one treatment over a period of days, weeks, months, or years. In various embodiments, the cells delivered to the patient are allogeneic with respect to the subject. The cancer can be any cancer, including, without limitation, a hematological cancer, bladder cancer, breast cancer, a chondrosarcoma, colorectal cancer, esophageal cancer, gastric cancer, a glioma, a glioblastoma, head and neck cancer, kidney cancer, a leiomyoma, a leiomyosarcoma, liver cancer, lung cancer, melanoma, mesothelioma, a neurocytoma, an osteosarcoma, ovarian cancer, pancreatic cancer, prostate cancer, rhabdosarcoma, renal cancer, testicular cancer, or uterine cancer.

In any of the afore-mentioned embodiments on the use of host cells, or on the host cell, or population of host cells for use, or on pharmaceutical compositions or their use, the cancer to be treated may be a hematological cancer, bladder cancer, breast cancer, a chondrosarcoma, colorectal cancer, esophageal cancer, gastric cancer, a glioma, a glioblastoma, head and neck cancer, kidney cancer, a leiomyoma, a leiomyosarcoma, liver cancer, lung cancer, melanoma, mesothelioma, a neurocytoma, an osteosarcoma, ovarian cancer, pancreatic cancer, prostate cancer, rhabdosarcoma, renal cancer, testicular cancer, or uterine cancer.

Provided herein is a method of treating a subject with cancer, comprising administering a pharmaceutical composition according to any disclosed herein to a subject with cancer. The host cell or population of host cells may optionally be allogeneic with respect to the subject. In various embodiments 10⁴ to 10¹¹ cells are administered to the subject. In various embodiments the cells are administered by injection or infusion and in various embodiments the cells are administered to the subject more than once.

In any of the treatments, uses, or pharmaceutical compositions disclosed herein, the cells may be allogeneic with respect to the subject. The cancer is a hematological cancer, bladder cancer, breast cancer, a chondrosarcoma, colorectal cancer, esophageal cancer, gastric cancer, a glioma, a glioblastoma, head and neck cancer, kidney cancer, a leiomyoma, a leiomyosarcoma, liver cancer, lung cancer, melanoma, mesothelioma, a neurocytoma, an osteosarcoma, ovarian cancer, pancreatic cancer, prostate cancer, rhabdosarcoma, renal cancer, testicular cancer, or uterine cancer.

A further aspect provides recombinant nucleic acid molecules. In various embodiments, a nucleic acid molecule encoding a chimeric scFv-TCRγ is provided, where the scFv-TCRγ subunit comprises, from the N-terminus to the C-terminus:
an scFv that binds a target antigen; and
(1) a combined connecting peptide, transmembrane domain, and intracellular domain sequence having at least 95% identity to SEQ ID NO:17; or
(2) a TCRγ connecting peptide having at least 95% identity to SEQ ID NO:9;
a TCRγ transmembrane domain having at least 95% identity to SEQ ID NO:10; and
a TCRγ intracellular domain having at least 95% identity to SEQ ID NO:11.

In any of the above embodiments of a nucleic acid molecule, sequence, or construct encoding an scFv-TCRγ polypeptide, the TCRγ connecting peptide may comprise, consist essentially of, or consist of a sequence having at least 95% identity to SEQ ID NO:9, the TCRγ transmembrane domain may comprise, consist essentially of, or consist of a sequence having at least 95% identity to SEQ ID NO:10, and/or the TCRγ intracellular domain may comprise, consist essentially of, or consist of a sequence having at least 95% identity to SEQ ID NO:11. In any of the above embodiments or those disclosed below of a nucleic acid molecule, sequence, or construct encoding an scFv-TCRγ polypeptide, the TCRγ connecting peptide may comprise, consist essentially of, or consist of SEQ ID NO:9, the TCRγ transmembrane domain may comprise, consist essentially of, or consist of SEQ ID NO:10, and/or the TCRγ intracellular domain may comprise, consist essentially of, or consist of a sequence having at least 95% identity to SEQ ID NO:11. In any of the above embodiments or those disclosed below of a nucleic acid molecule, sequence, or construct encoding an scFv-TCRγ polypeptide, the scFv-TCRγ subunit can comprise, consist essentially of, or consist of a sequence having at least 95% identity to SEQ ID NO:17. In any of the above embodiments or those disclosed below of a nucleic acid molecule, sequence, or construct encoding an scFv-TCRγ polypeptide, the scFv-TCRγ subunit can comprise, consist essentially of, or consist of SEQ ID NO:17.

In some embodiments of a recombinant nucleic acid molecule of chimeric scFv-TCRγ, wherein the target antigen is PD-L1. For example, in some embodiments of a recombinant nucleic acid molecule encoding a chimeric scFv-TCRγ, the chimeric scFv-TCRγ comprises a combined scFv, TCRγ connecting peptide, TCRγ transmembrane domain, and TCRγ intracellular domain sequence having at least 95% identity to SEQ ID NO:25.

In some embodiments of a recombinant nucleic acid molecule encoding an scFv-TCRγ that binds PDL1, the encoded scFv-TCRγ comprises an scFv with a heavy chain variable region comprising a heavy chain complementarity determining region (HCDR1) comprising SEQ ID NO:46, an HCDR2 comprising SEQ ID NO:47, and an HCDR3 comprising SEQ ID NO:48, and a light chain variable region comprising a light chain complementarity determining region (LCDR1) comprising SEQ ID NO:49, an LCDR2 comprising SEQ ID NO:50, and an LCDR3 comprising SEQ ID NO:51.

In some embodiments of a recombinant nucleic acid molecule encoding an scFv-TCRγ that binds PDL1, the encoded scFv-TCRγ comprises a heavy chain variable region having at least 95% identity to SEQ ID NO:1 and a light chain variable region having at least 95% identity to SEQ ID NO:2, optionally wherein the heavy chain variable region comprises the sequence of SEQ ID NO:1 and the light chain variable region comprises the sequence of SEQ ID NO:2.

In some embodiments of a recombinant nucleic acid molecule encoding an scFv-TCRγ that binds PDL1, the encoded scFv comprises an amino acid sequence having at least 95% identity to SEQ ID NO:4, optionally wherein the scFv comprises the sequence of SEQ ID NO:4.

In further embodiments of a nucleic acid molecule encoding a chimeric scFv-TCRγ as provided herein, the target antigen is a tumor associated antigen.

In some embodiments of the host cell or population of host cells described herein, the target antigen bound by the scFv of the scFv-TCRγ is CD19. In some embodiments, a host cell or population of host cells that includes a nucleic acid sequence encoding an scFv-TCRγ as set forth in Embodiment 1, where the scFv-TCRγ subunit comprises a combined scFv. TCRγ connecting peptide, TCRγ transmembrane domain, and TCRγ intracellular domain sequence having at least 95% identity to SEQ ID NO:62. In some embodiments, the nucleic acid molecule encodes a CD19 targeting scFv-TCRγ, where the scFv comprises a heavy chain variable region having at least 95% identity to SEQ ID NO:58 and a light chain variable region having at least 95% identity to SEQ ID NO:59, optionally wherein the heavy chain variable region comprises the sequence of SEQ ID NO:58 and the light chain variable region comprises the sequence of SEQ ID NO:59. In some embodiments, the nucleic acid molecule encodes an scFv-TCRγ comprising an amino acid sequence having at least 95% identity to SEQ ID NO:60, and optionally comprising the sequence of SEQ ID NO:60.

The recombinant nucleic acid molecule of any of the embodiments above or described herein can comprise a vector. The recombinant nucleic acid molecule of any of the embodiments described herein can be a linear DNA molecule.

In various embodiments a nucleic acid molecule as disclosed herein that encodes an scFv-TCRγ further includes a promoter operably linked to the sequence encoding the scFv-TCRγ subunit.

A recombinant nucleic acid molecule as provided herein in some embodiments comprises homology arms for insertion into a locus of the human genome. For example, the recombinant nucleic acid molecule can comprise homology arms for insertion into the TRAC or TRBC locus.

### BRIEF DESCRIPTION OF THE DRAWINGS

FIGS. 1A-B provide schematic illustrations of engineered scFv-TCRγδ receptors situated in a cell membrane. FIG. 1A depicts an engineered scFv-δ-TCRγδ and a CAR that includes the CD3 signaling domain and a 4-1BB co-signaling domain. FIG. 1B depicts an engineered scFv-δ-TCRγδ (scFv on the 8 polypeptide) and an engineered scFv-γ-TCRγδ (scFv on the γ polypeptide).

FIGS. 2A-B provide diagrams of constructs that encode PD-L1 scFv-TCRγδs: FIG. 2A: αPDL1-δ-TCRγδ receptor construct GD102 that encodes an N-terminally truncated TCRγ (NT-TCRγ) polypeptide and an αPDL1-TCRδ polypeptide linked via a T2A sequence, where each subunit polypeptide includes an N-terminal signal sequence; FIG. 2B: αPDL1-γ-TCRγδ receptor construct GD109 that encodes an αPDL1-TCRγ polypeptide and an N-terminally truncated TCRδ (NT-TCRδ) polypeptide linked via a T2A sequence, where each subunit polypeptide includes an N-terminal signal sequence. CP, connecting peptide; TMD, transmembrane domain; ICD, intracellular domain. Diagrams are not to scale.

FIGS. 3A-3D provide the results of flow cytometry of T cells 14 days after transfection with engineered TCR constructs GD102 (αPDL1-δ-TCRγδ construct), and GD109 (αPDL1-γ-TCRγδ construct) and of non-transfected activated T cells (ATC). Also shown are TRAC knockout cells (KO) that like the GD102 and GD109 cells have a disrupted TCRα gene but were not transfected with a nucleic acid construct encoding an engineered receptor. FIG. 3A: staining with antibodies recognizing αβTCR (x axis) and CD3ε (y axis); FIG. 3B: staining with antibodies recognizing the variable domain of a γ (x axis) and δ (y axis) TCR subunit; FIG. 3C: staining with antibodies recognizing CD8 (x axis) and CD4 (y axis); FIG. 3D: staining with PD-L1-Fc (x axis).

FIGS. 4A and 4B provide the results of flow cytometry of T cells 10 days after transfection of non-transfected activated T cells (ATC), TRAC knockout cells that were not transfected with a nucleic acid construct (TRAC KO), T cells transfected with a PDL1 CAR construct, T cells transfected with the GD102 construct (αPDL1-δ-TCRγδ), and T cells transfected with the GD109 construct (αPDL1-γ-TCRγδ), as described in Example 5. FIG. 4A: flow cytometry analysis of untransfected (UT), TRAC knockout (TRAC KO), PDL1 CAR-transfected, and GD102 (αPDL1-δ-TCRγδ)-transfected cells. Upper panels, staining with PDL1-Fc; Lower panels, staining with a CD3 antibody (y axis) and a TCR antibody (x axis). FIG. 4B: flow cytometry analysis of TRAC knockout (TRAC KO), GD102 (αPDL1-δ-TCRγδ)-transfected, and GD109 (αPDL1-γ-TCRγδ)-transfected cells. Upper panels, staining with PDL1-Fc; Lower panels, staining with a CD3 antibody (y axis) and a TCR antibody (x axis).

FIG. 5 provides the results of flow cytometry of αPDL1 CAR cells and αPDL1-δ-TCRγδ cells stained to detect expression of CD45A, CCR7, and CD62L after gating for expression of CD4 (right panels) or CD8 (right panels) thirteen days after transfection.

FIG. 6A shows the results of flow cytometry to analyze expression of degranulation marker CD107a and Granzyme B expression in response to co-culturing with target cells. PDL1 CAR-T cells or αPDL1-δ-TCRδδ⁻T cells were cultured alone or cocultured with either wild type A549 cells (549 WT) or A549 PDL1-knockout cells (A549 KO) in the presence of Brefeldin A. Representative CD107a and granzyme B expression gated on CD8⁺ cells are shown. Shifted curves illustrate the CD107a or Granzyme B expression after A549 cell stimulation. FIG. 6B is a graph showing representative cytotoxic activity of PDL1 CAR and δ-TCRγδ-T cells. CAR or δ-TCRγδ expressing T cells at indicated E:T ratios were added to the A549 tumor cell culture. Cytotoxicity (percentage of lysis) was examined after 4 h of co-culturing by staining with fixable viability dye and annexin V. FIG. 6C: secreted IFN-γ levels. FIG. 6D: GM-CSF levels by CAR- or δ-TCRγδ-T cells. CAR or δ-TCRγδ expressing T cells were incubated with A549 WT or A549 KO cells at 1:1 ratio and supernatant was collected after overnight incubation. The cytokine levels were measured by ELISA. All representative data shown are from cells of different donors and experiments were repeated using cells of at least 3 different donors. All data are mean±SEM.

FIG. 7A shows the results of flow cytometry to analyze expression of degranulation marker CD107a and Granzyme B expression by FACS. TRAC knockout, PDL1 CAR-T cells or αPDL1-δ-TCRγδ T cells were cultured alone or cocultured with SK-MEL-5 cells in the presence of Brefeldin A. Representative CD107a and granzyme B expression gated on CD8⁺ cells are shown. Shifted curves illustrate the CD107a or Granzyme B expression after SK-MEL-5 cells stimulation. FIG. 7B shows the results of representative cytotoxicity assays with PDL1 CAR-T cells and αPDL1-δ-TCRδδ⁻T cells added to SK-MEL-5 cell cultures at the indicated ratios. Cytotoxicity was examined after 4 h of co-culturing by staining with fixable viability dye and annexin V. Representative data are shown using cells of different donors. All data are mean±SEM.

FIG. 8A-F provides the results of in vivo studies using αPDL1-δ-TCRδδ⁻T cells and PDL1 CAR-T cells. FIGS. 8A and 8B: A549 WT-bearing NSG mice were treated with 1×10⁷ TRAC-CAR (n=10) or TRAC-δ-TCRγδ T cells (n=10), as well as equal cell number of TRAC-KO T cells as a control group (n=10). Tumor size (A) was measured at indicated time points and analyzed through a 46-day period. Peripheral blood was drawn at indicated time points and numbers of CD45⁺ cells (B) were calculated. FIGS. 8C and 8D: SK-MEL-5 bearing NSG mice were received 1×10⁷ TRAC-KO (n=8), or TRAC-δ-TCRγδ T cells (n=8). Tumor size (C) was measured at indicated time points and analyzed through a 40-day period. Peripheral blood was drawn at indicated time points and numbers of CD45⁺ cells (D) were calculated. FIGS. 8E and 8F: MDA-MB-231 bearing NSG mice were received 1×10⁷ TRAC-KO (n=8), or TRAC-δ-TCRγδ T cells (n=8). Tumor size (E) was measured at indicated time points and analyzed through a 43-day period. Peripheral blood was drawn at indicated time points and numbers of CD45+ cells (F) were calculated. All experiments were repeated in cells from 2 different donors. All data are mean±SEM.

FIG. 9A shows degranulation marker CD107a expression and FIG. 9B shows granzyme B expression by αPDL1-δ-TCRγδ-T cells and αPDL1-γ-TCRγδ-T cells co-cultured with the indicated tumor cells (A549 PDL1-knockout, A549 wild type, MDA-MB231, and SK-MEL-5) in the presence of Brefeldin A. CD107a and granzyme B were examined by FACS. Representative CD107a and granzyme B expression gated on CD8+ cells are shown.

FIGS. 10A-B provide graphs showing expansion of T cells expressing a PDL1 CAR, αPDL1-δ-TCRγδ, and αPDL1-γ-TCRγδ on wild type A549 (A549WT) tumor cells (right portion of graphs) but not on A549 KO (PD-L1 Null) tumor cells (left portion of graphs). 10A) A549 KO and A549 WT stimulation with an effector to target ratio (E:T) of 1:2. 10B) A549 KO and A549 WT stimulation with an E:T of 1:4

FIGS. 11A-E show the results of real time impedance-based cytotoxicity assays using A549 wild type (WT) cells as targets. FIG. 11A: ATC, activated (nontransformed) T cells as effectors; FIG. 11B: KO, T cells having a disrupted TRAC gene but lacking an engineered receptor construct as effectors; FIG. 11C: T cells engineered to express the PD-L1 CAR construct as effectors; FIG. 11D: T cells engineered to express the GD102 (αPDL1-δ-TCRγδ) construct as effectors; FIG. 11E: T cells engineered to express the GD109 (αPDL1-γ-TCRγδ) construct as effectors.

FIGS. 12A-E provide graphs of cell index based on real time impedance-based cytotoxicity assays using A549 knockout (KO) cells as targets. FIG. 12A: ATC, activated (nontransformed) T cells as effectors; FIG. 12B: KO, T cells having a disrupted TRAC gene but lacking an engineered receptor construct as effectors; FIG. 12C: T cells engineered to express the PD-L1 CAR construct as effectors; FIG. 12D: T cells engineered to express the GD102 (αPDL1-δ-TCRγδ) construct as effectors; FIG. 12E: T cells engineered to express the GD109 (αPDL1-γ-TCRγδ) construct as effectors.

FIGS. 13A-E shows the results of real time impedance-based cytotoxicity assays using MB231 wild type (WT) cells as targets. FIG. 13A: ATC, activated (nontransformed) T cells as effectors; FIG. 13B: KO, T cells having a disrupted TRAC gene but lacking an engineered receptor construct as effectors; FIG. 13C: T cells engineered to express the PD-L1 CAR construct as effectors; FIG. 13D: T cells engineered to express the GD102 (αPDL1-δ-TCRγδ) construct as effectors; FIG. 13E: T cells engineered to express the GD109 (αPDL1-γ-TCRγδ) construct as effectors.

FIGS. 14A-D show production of cytokines by T cells co-cultured with wild type and PDL1 knockout (KO) A549 tumor cells. FIGS. 14A and 14C provide graphs showing the amount of interferon gamma (IFNγ) secreted by T cells expressing the PD-L1 CAR, GD102 (scFv-δ-TCRγδ) construct, and GD109 (scFv-γ-TCRγδ) construct after overnight co-culturing with A549 PDL1 knockout and wild type cells at various effector to target ratios (E:T). FIGS. 14B and 14D provide graphs showing the amount of GM-CSF secreted by T cells expressing the PD-L1 CAR, GD102 (scFv-δ-TCRγδ) construct, and GD109 (scFv-γ-TCRγδ) construct after overnight co-culturing with A549 PDL1 knockout and wild type cells at various effector: target ratios (Example 11). Cytokine release into the supernatant was evaluated by ELISA.

FIGS. 15A and 15B provide graphs showing the amount of IFNγ (15A) and GM-CSF (15B) secreted by T cells expressing the PD-L1 CAR, T cells expressing the GD102 (αPDL1-δ-TCRγδ) construct, and T cells expressing the GD109 (αPDL1-γ-TCRγδ) construct. MDA-MB231 cells were co-incubated with T cells overnight at a 1:1 ratio and cytokine release into the supernatant was evaluated by ELISA.

FIGS. 16A-D provide tumor volumes and transferred CD45+ cell counts over time for A549 WT tumor-bearing NSG mice that received different numbers of αPDL1-δ-TCRγδ⁻T cells (GD102, n=10 per dosage group) or αPDL1-γ-TCRγδ⁻T cells (GD109, n=10 per dosage group), as well as equal number of TRAC-KO T cells (n=10) as a control group. Tumor size (A and B) was measured at indicated time points and analyzed through a 68-day period. Peripheral blood was analyzed at indicated time points and numbers of CD45⁺ cells (C and D) were calculated. For in vitro assays, all representative data shown are from different donors and experiments were repeated using cells from at least 3 different donors. For in vivo studies, all experiments were repeated in cells from 2 different donors.

FIG. 17A-B provide the results of a tumor infiltration study. A549 WT tumor-bearing NSG mice received 1×10⁷ of αPDL1-γ-TCRγδ⁻T cells (n=3) or TRAC-KO T cells (n=3) as control. On indicated days, subcutaneous tumors were removed to analyze the T cells within the tumors. Numbers of CD45⁺ T cells in the tumor site evaluated by (A) flow cytometry and (B) representative in situ staining of CD45⁺ T cells (Brown staining) within the tumors.

FIG. 18A provides a schematic diagram of rechallenge of A549 WT cells in NSG mice in which A549 WT-bearing NSG mice were initially treated with 1×10⁷ γ-TCRγδ T cells (n=10). FIGS. 18B-C: After tumor elimination, mice were rechallenged with A549 WT cells, peripheral blood was drawn at indicated time points and the number of CD45⁺ cells (B) was calculated. Tumor size (C) was measured at indicated time points after rechallenging and the % of CD45RA−, CCR7− cells were analyzed.

FIG. 19 provides the results of an in vivo tissue infiltration study. A549 WT tumor-bearing NSG mice received 1×10⁷ of αPDL1-γ-TCRγδ⁻T cells (n=3) or TRAC-KO T cells (n=3) as control. On indicated days, various tissues were removed to analyze the T cells. Numbers of CD45⁺ T cells in the tissues were evaluated by flow cytometry.

FIGS. 20A-B show the open reading frames of nucleic acid molecules encoding engineered PD-L1 scFv-TCR polypeptides. FIG. 20A: αPD-L1 scFv-TCRγ polypeptide precursor that includes an N-terminal signal sequence. FIG. 20B: αPD-L1 scFv-TCRδ polypeptide precursor that includes an N-terminal signal sequence. CP, connecting peptide; TMD, transmembrane domain; ICD, intracellular (cytoplasmic) domain. Diagrams are not to scale.

FIG. 21A provides schematics of ScFv-TCRγδ receptors and ScFv-TCRγ and ScFv-TCRδ chains in the membrane of a cell. The leftmost diagram shows an ScFv-γ-TCRγδ receptor and a ScFv-δ-TCRγδ receptor. To the right of the arrow is an ScFv-TCRγ chain and an ScFv-TCRδ chain envisioned in the absence of partner TCR core subunits. FIG. 21B provides the results of flow cytometry with labeled PDL1 to detect engineered receptors expressed by cell populations transfected with a construct encoding an αPDL1-TCRγ (left) and an αPDL1-TCRδ (right).

FIGS. 22A-B show the results of real time impedance-based cytotoxicity assays using A549 cells as targets and cells expressing the αPDL1-TCRγ polypeptide as effectors as described in Example 15. A) Real time cytotoxicity assay using A549 PDL1 knockout (KO) cells as targets; B) Real time cytotoxicity assay using A549 wild type cells as targets. The earliest descending curve is the assay at a 5:1 ratio, followed by the 1:1 ratio curve and then the 1:5 ratio curve descends last.

FIGS. 23A-B show the results of real time impedance-based cytotoxicity assays using A549 wild type cells as targets and either A) αPDL1-γ-TCRγδ-T cells or B) αPDL1-TCRγ-T cells as effectors as described in Example 15.

FIGS. 24A-B show the tumor size and circulating transferred T cells after A549 WT tumor-bearing NSG mice were treated with 1×10⁷ TRAC-KO (n=6) or αPDL1-TCRγ T cells (n=6). Tumor size (A) was measured at indicated time points and analyzed through a 15-day period. Peripheral blood was drawn at indicated time points and numbers of CD45⁺ cells (B) were calculated. All representative data shown are using cells from different donors and all in vitro experiments were repeated in cells from at least 3 different donors.

FIG. 25 shows the results of an expansion assay based on dilution of the CTV dye. αCD19-TCRγ-T cells, CD19 CAR-T cells, and TCR knockout (KO) T cells from two different donors that had been loaded with CTV dye were co-cultured with Nalm-6 tumor cells expressing the GFP gene in the absence of IL-2. The figure shows the results of flow cytometry detecting CTV in GFP negative cells.

FIG. 26 provides the results of cytotoxicity assays where the effectors were αCD19 scFv-TCRγ-T cells (effector cells), CD19 CAR-T cells, or TRAC knockout (KO) T cells and the target cells were CD19-expressing K562 cells. Annexin V was detected as a marker of cyolysis. Left panels show viable cells and right panels show percentage of lysed cells after four hours (upper graphs) or overnight (lower graphs) incubation.

FIG. 27 provides the results of cytotoxicity assays where the effectors were αCD19 scFv-TCRγ-T cells (effector cells), CD19 CAR-T cells, or TRAC knockout (KO) T cells and the target cells were Nalm6 cells. Annexin V was detected as a marker of cyolysis. Left panels show viable cells and right panels show percentage of lysed cells after four hours (upper graphs) or overnight (lower graphs) incubation.

FIG. 28 provides the results of cytokine detection assays on cocultures of αCD19-TCRγ-T cells, CD19 CAR-T cells, and TRAC knockout (KO) T cells with target cells, 1×10⁵ effector cells were cocultured with 1×10⁵ an equal number of Nalm6 tumor cells in 96-well plate at 37° C. overnight. Supernatants were collected and cytokines were measured using IFN-γ and GM-CSF ELISA kits (Thermo Fisher) and read by Cytation5 imaging reader. FIG. 28 shows that the αCD19-TCRγ-T cells produced somewhat less interleukin 2 (IL-2) and interferon gamma (IFN-g) and slightly more tumor necrosis factor alpha (TNF-α) than was produced by CD19 CAR-T cells. Notably, when co-cultured with Nalm6 target cells, the amount of GM-CSF produced by the αCD19-TCRγ-T cells was much lower than that produced by CAR-T cells.

FIG. 29 provides in vivo images of mice inoculated with Nalm6 CD19+ tumor cells for four weeks after treatment with αCD19-TCRγ-T cells and CD19 CAR-T cells as described in Example 20.

FIGS. 30A and B provide graphs of tumor size and body weight for mice inoculated with Nalm6 CD19+ tumor cells for four weeks after treatment with αCD19-TCRγ-T cells and CD19 CAR-T cells as described in Example 20.

FIG. 31 provides graphs of IFN-γ and GM-CSF in the blood of mice inoculated with Nalm6 CD19+ tumor cells in the first 11 days after treatment with αCD19-TCRγ-T cells and CD19 CAR-T cells as described in Example 20. Cytokines were detected in diluted serum collected from mice by ELISA.

### DETAILED DESCRIPTION

Molecular biology and biochemical techniques employed herein are generally performed according to conventional procedures well known in the art and as described in various general and more specific references that are cited and discussed herein unless otherwise indicated. Sec, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992). A number of basic texts describe standard antibody production processes, including, Borrebacck (ed) Antibody Engineering, 2nd Edition Freeman and Company, NY, 1995; McCafferty et al. Antibody Engineering, A Practical Approach IRL at Oxford Press, Oxford, England, 1996; and Paul (1995) Antibody Engineering Protocols Humana Press, Towata, N.J., 1995; Paul (ed.), Fundamental Immunology, Raven Press, N.Y, 1993; Coligan (1991) Current Protocols in Immunology Wiley/Greene, NY; Harlow and Lane (1989) Antibodies: A Laboratory Manual Cold Spring Harbor Press, NY; Stites et al. (eds.) Basic and Clinical Immunology (4th ed.) Lange Medical Publications, Los Altos, Calif., and references cited therein; Coding Monoclonal Antibodies: Principles and Practice (2nd ed.) Academic Press, New York, N. Y., 1986, and Kohler and Milstein Nature 256:495-497, 1975.

Enzymatic reactions and enrichment/purification techniques are also well known and are performed according to manufacturer's specifications, as commonly accomplished in the art or as described herein. Standard techniques can be used for chemical syntheses, chemical analyses, pharmaceutical preparation, formulation, and delivery, and treatment of patients.

Throughout this application various publications, patents, and/or patent applications are referenced. The disclosures of each of the publications, patents and/or patent applications cited herein is hereby incorporated by reference in its entirety into this application in order to more fully describe the state of the art to which this disclosure pertains. In the event that any document or other material incorporated by reference contradicts any explicit content of this specification, including definitions, this specification controls.

The headings provided herein are not limitations of the various aspects of the disclosure, which aspects can be understood by reference to the specification as a whole.

### Definitions

Unless defined otherwise, technical and scientific terms used herein have meanings that are commonly understood by those of ordinary skill in the art unless defined otherwise. Generally, terminologies pertaining to techniques of cell and tissue culture, molecular biology, immunology, microbiology, genetics, transgenic cell production, protein chemistry and nucleic acid chemistry and hybridization described herein are well known and commonly used in the art. The terminology used in connection with, and the laboratory procedures and techniques of, analytical chemistry, synthetic organic chemistry, and medicinal and pharmaceutical chemistry described herein are also well known and commonly used in the art.

Unless otherwise required by context herein, singular terms shall include pluralities and plural terms shall include the singular. Singular forms “a”, “an” and “the”, and singular use of any word, include plural referents unless expressly and unequivocally limited on one referent.

It is understood the use of the alternative (e.g., “or”) herein is taken to mean either one or both or any combination of the alternatives.

The term “and/or” used herein is to be taken mean specific disclosure of each of the specified features or components with or without the other. For example, the term “and/or” as used in a phrase such as “A and/or B” herein is intended to include “A and B”, “A or B”, “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, terms “comprising”, “including”, “having” and “containing”, and their grammatical variants, are intended to be non-limiting so that one item or multiple items in a list do not exclude other items that can be added to the listed items. It is understood that wherever aspects or embodiments are described herein with the language “comprising,” otherwise analogous aspects or embodiments described in terms of “consisting of” and/or “consisting essentially of” are also provided. That is, the term “comprising” also encompasses “consisting essentially of” and “consisting of”. “Consisting essentially of” limits the scope of a claim to the specified materials or steps “and those that do not materially affect the basic and novel characteristic(s)” of the claimed invention. [MPEP 2111.03]. In a specific example, the description of engineered receptor polypeptides herein, “consisting essentially of” should not be construed as excluding the possibility of linkers that may connect described regions of the engineered receptor polypeptides. As nonlimiting examples, a peptide linker may be positioned between an ScFv and a connecting peptide or may be positioned between a connecting peptide and a transmembrane domain.

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “approximately” can mean within one or more than one standard deviation per the practice in the art. Alternatively, “about” or “approximately” can mean a range of up to 10% (i.e., ±10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “approximately” should be assumed to be within an acceptable error range for that particular value or composition.

As used herein “T cell receptor” or “TCR”, unless otherwise specifying an alternative composition, refers to a T cell receptor that includes core subunit polypeptides (either TCRα and TCRβ or TCRγ and TCRδ). Analogously, an “scFv-TCR” includes core subunits of a scFv-TCR as disclosed herein (for example, a chimeric scFv-TCR& polypeptide and an N-terminally truncated TCRγ or a chimeric scFv-TCRγ polypeptide and an N-terminally truncated TCRδ). A “T cell receptor complex” or “TCR complex” is used to refer to the core TCR polypeptides (e.g., a TCRα/TCRβ dimer, an scFv-TCRγ/NT-TCRδ dimer, etc.) in association with a CD3ζζ dimer, a CD3γε dimer, and a CD3δε dimer. The terms “TCR” and “TCR complex” may be used to refer to endogenous T cell receptors and T cell receptor complexes or to T cell receptors and T cell receptor complexes that incorporate subunit polypeptides based on engineered TCR subunits (e.g., scFv-TCRγ, NT-TCRδ, scFv-TCRδ, NT-TCRγ). In general, native (unmodified) TCR subunits may be referred to herein as TCR chains or simply by the chain designation (e.g., “a TCRα chain” or “TCRα”), and engineered TCR subunits may be referred to as TCR polypeptides or simply by the polypeptide designation (e.g., “an scFv-TCRγ polypeptide” or “scFv-TCRγ”).

The term “native” is used herein to mean a biomolecule (e.g., a polypeptide or nucleic acid sequence) that has the same sequence or chemical structure as the naturally occurring biomolecule, and more particularly is used to describe a polypeptide having the same amino acid sequence as a naturally-occurring polypeptide or to describe a nucleic acid sequence having the same nucleotide sequence as a naturally-occurring nucleic acid sequence.

The term “endogenous” is used herein to refer a naturally occurring biomolecule or other component of an organism or tissue. For example, an endogenous polypeptide is a polypeptide produced by a cell from a naturally-occurring gene that has not been modified by technical intervention (e.g., gene editing).

An “exogenous” nucleic acid molecule or gene is one that does not occur naturally in a cell but has been introduced into the cell or a progenitor cell, for example by transfection or transduction. An exogenous nucleic acid sequence, molecule, or gene may also be referred to as a “transgene” or as an “introduced” or “non-native” nucleic acid sequence, molecule, or gene.

A nucleic acid or polypeptide (amino acid) sequence is “derived from” another sequence, such as a native nucleic acid or polypeptide sequence, when it exhibits at least 60%, at least 65%, at least 70%, least 75%, at least 80%, least 85%, at least 90%, at least 95%, at least 96%, at least 97%, at least 98%, or at least 99% identity to the reference sequence. Alternatively, a nucleic acid or polypeptide (amino acid) sequence that is “derived from” another sequence may have one or more deletions or insertions with respect to the reference sequence, for example, may be an polypeptide domain derived from a reference polypeptide or may be a truncated or tagged polypeptide derived from a reference polypeptide.

As used herein, a first sequence is considered to “have at least X % identity to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine as a complement). Similarly, for polypeptides, post-translational modification of amino acid residues does not detract from sequence identity. Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server are generally appropriate.

The terms “peptide”. “polypeptide”, “polypeptide chain” and “protein” and other related terms used herein are used interchangeably and refer to a polymer of amino acids and are not limited to any particular length. Polypeptides may comprise natural and non-natural amino acids. Polypeptides include recombinant or chemically-synthesized forms. Polypeptides also include precursor molecules and mature molecules. Precursor molecules include those that have not yet been subjected to post-translation modification such as proteolytic cleavage (including cleavage of a signal peptide directing secretion or membrane insertion of a polypeptide), cleavage due to ribosomal skipping (e.g., mediated by a self-cleaving cleaving sequence such as for example T2A, P2A, E2A or F2A; Donelly et al. (2001) J. Gen. Virol. 82:1013-25; Sharma et al. (2012) Nucl. Acids Res. 40:3143-51), hydroxylation, methylation, lipidation, acetylation, SUMOylation, ubiquitination, glycosylation, fucosylation, phosphorylation, disulfide bond formation, processing of a secretory signal peptide or non-enzymatic cleavage at certain amino acid residues. Polypeptides include mature molecules that have undergone any one or any combination of the post-translation modifications described above. These terms encompass native proteins, recombinant proteins and artificial proteins, protein fragments and polypeptide analogs (such as muteins, variants, chimeric proteins and fusion proteins) of a protein sequence as well as post-translationally, or otherwise covalently or non-covalently, modified proteins. Two or more polypeptides (e.g., 2-6 or more polypeptide chains) can associate with each other, via covalent and/or non-covalent association, to form a polypeptide complex. Association of the polypeptide chains can also include peptide folding. Thus, a polypeptide complex can be dimeric, trimeric, tetrameric, or higher order complexes depending on the number of polypeptide chains that form the complex.

The terms “nucleic acid”, “polynucleotide” and “oligonucleotide” and other related terms used herein are used interchangeably and refer to polymers of nucleotides and are not limited to any particular length. The length of a nucleic acid may be referred to in base pairs or nucleotides, which may be used interchangeably regardless of whether a nucleic acid is single-stranded or double-stranded. Nucleic acids include recombinant and chemically-synthesized forms. Nucleic acids include DNA molecules (cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. Nucleic acid molecule can be single-stranded or double-stranded. In one embodiment, the nucleic acid molecules of the disclosure comprise a contiguous open reading frame encoding at least one engineered TCR polypeptide, or a fragment, derivative, mutein, or variant thereof. In one embodiment, nucleic acids comprise one type of polynucleotide or a mixture of two or more different types of polynucleotides. Nucleic acids encoding engineered T cell receptors (e.g., scFv-TCRs) and their component subunits, such as scFv-TCR polypeptides are described herein. With respect to embodiments involving a first nucleic acid (e.g., encoding a first polypeptide) and a second nucleic acid (e.g., encoding a second polypeptide), the first nucleic acid and second nucleic acid may be provided either as separate molecules or within the same continuous molecule (e.g., a plasmid or other construct containing first and second coding sequences).

The term “recover” or “recovery” or “recovering”, and other related terms, refers to obtaining a protein, including a protein complex (e.g., an scFv-TCR), a subunit thereof, from host cell culture medium or from host cell lysate or from the host cell membrane. In one embodiment, the protein is expressed by the host cell as a recombinant protein fused to a secretion signal peptide (leader peptide sequence) sequence which mediates secretion of the expressed protein from a host cell (e.g., from a mammalian host cell). The secreted protein can be recovered from the host cell medium. In one embodiment, the protein is expressed by the host cell as a recombinant protein that lacks a secretion signal peptide sequence which can be recovered from the host cell lysate. In one embodiment, the protein is expressed by the host cell as a membrane-bound protein which can be recovered using a detergent to release the expressed protein from the host cell membrane. In one embodiment, irrespective of the method used to recover the protein, the protein can be subjected to procedures that remove cellular debris from the recovered protein. For example, the recovered protein can be subjected to chromatography, gel electrophoresis and/or dialysis. In one embodiment, the chromatography comprises any one or any combination or two or more procedures including affinity chromatography, hydroxyapatite chromatography, ion-exchange chromatography, reverse phase chromatography and/or chromatography on silica.

The term “isolated” refers to a protein or protein complex (e.g., an scFv-TCR, a subunit thereof, or a precursor polypeptide thereof) or polynucleotide that is substantially free of other cellular material. A protein may be rendered substantially free of naturally associated components (or components associated with a cellular expression system or chemical synthesis methods used to produce the polypeptide or complex) by isolation, using protein purification techniques well known in the art. The term isolated also refers in some embodiment to protein or polynucleotides that are substantially free of other molecules of the same species, for example other protein or polynucleotides having different amino acid or nucleotide sequences, respectively. The purity of homogeneity of the desired molecule can be assayed using techniques well known in the art, including low resolution methods such as gel electrophoresis and high resolution methods such as HPLC or mass spectrometry. An isolated protein, protein complex, or nucleic acid molecule is also considered isolated when it is removed from its cellular milieu and substantially free of naturally associated components (or components associated with enzymatic or chemical synthesis methods used to produce the nucleic acid molecule) by isolation, using nucleic acid purification techniques well known in the art. In various embodiments, the disclosure provides isolated precursor polypeptides, scFv-TCR subunits, scFv-TCRs and scFv-TCR complexes, and nucleic acid molecules encoding any of the foregoing.

The term “precursor polypeptide(s)” or related terms, may be used herein to refer to a precursor polypeptide that can be processed to become a first and/or second polypeptide chain that associates/assembles to form an scFv-TCR. In any of the precursor polypeptide embodiments described herein that comprise a “self-cleaving” sequence, the self-cleaving sequence may be a 2A sequence, such as a T2A, P2A, E2A, or F2A sequence (SEQ ID NOs: 12, 52, 53, and 54, respectively). The precursor polypeptide can be processed by producing first and second polypeptide chains that may be inserted into the cell membrane A precursor polypeptide can alternatively or in addition include one or more signal peptides that can be cleaved on insertion of a first polypeptide into the cell membrane and/or secretion of a second polypeptide from the cell. The first and second scFv-TCR polypeptides can assemble together, for example can associate via a disulfide bond between extracellular regions of the first and second scFv-TCR polypeptides, in an scFv-TCR complex.

The term “signal peptide”, “secretion signal peptide”, “leader sequence”, “leader peptide”, or “peptide signal sequence” or refers to a peptide sequence that is located at the N-terminus of a polypeptide. A signal peptide directs a polypeptide chain to a cellular secretory pathway and can direct integration and anchoring of the polypeptide into the lipid bilayer of the cellular membrane. Typically, a signal peptide is about 10-50 amino acids in length. A signal peptide can direct transport of a precursor polypeptide to the endoplasmic reticulum, for example as part of the biosynthetic pathway of a membrane protein or secreted protein. Any of various signal peptides can be incorporated into an scFv-TCR polypeptide precursor to direct the polypeptide to the cell membrane. In some embodiments, a signal peptide can comprise CD8α, CD28, or CD16 leader sequences. In some embodiments, the signal sequence comprises a mammalian sequence, including for example mouse or human Ig gamma secretion signal peptide. In some embodiments of the constructs described herein that encode precursors of receptor polypeptides such as scFv-TCR polypeptide precursors, a leader sequence comprises a mouse Ig gamma leader peptide sequence (SEQ ID NO:22), or a signal peptide provided herein as SEQ ID NO:8, SEQ ID NO:13, SEQ ID NO:23, SEQ ID NO:55, SEQ ID NO:56, or SEQ ID NO:57, as nonlimiting examples.

An “antigen binding protein” and related terms used herein refers to a protein comprising a portion that binds to an antigen, e.g., specifically binds an antigen, and, optionally, a scaffold or framework portion that allows the antigen binding portion to adopt a conformation that promotes binding of the antigen binding protein to the antigen. Examples of antigen binding proteins include chimeric antigen receptors (CARs), the scFv-TCRγ polypeptides disclosed herein, antibodies, including single chain antibodies (scFvs) and antibody fragments (e.g., an antigen binding portion of an antibody), antibody derivatives, and antibody analogs. The antigen binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding protein as well as wholly synthetic scaffolds comprising, for example, a biocompatible polymer. See, for example, Korndorfer et al., 2003, Proteins: Structure, Function, and Bioinformatics, Volume 53, Issue 1:121-129; Roque et al., 2004, Biotechnol. Prog. 20:639-654. In addition, peptide antibody mimetics (“PAMs”) can be used, as well as scaffolds based on antibody mimetics utilizing fibronection components as a scaffold.

An antigen binding protein can have, for example, the structure of an immunoglobulin. In one embodiment, an “immunoglobulin” refers to a tetrameric molecule composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa or lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The heavy and/or light chains may or may not include a leader sequence (signal sequence) for secretion. The variable regions of each light/heavy chain pair form the antibody binding site such that an intact immunoglobulin has two antigen binding sites. In one embodiment, an antigen binding protein can be a synthetic molecule having a structure that differs from a tetrameric immunoglobulin molecule but still binds a target antigen or binds two or more target antigens. For example, a synthetic antigen binding protein can comprise antibody fragments, 1-6 or more polypeptide chains, asymmetrical assemblies of polypeptides, or other synthetic molecules.

The variable regions of immunoglobulin chains exhibit the same general structure of relatively conserved framework regions (FR) joined by three hypervariable regions, also called complementarity determining regions or CDRs. From N-terminus to C-terminus, both light and heavy chains comprise the segments FR1, CDR1, FR2, CDR2, FR3, CDR3 and FR4.

One or more CDRs may be incorporated into a molecule either covalently or noncovalently to make it an antigen binding protein. An antigen binding protein may incorporate the CDR(s) as part of a larger polypeptide chain, may covalently link the CDR(s) to another polypeptide chain, or may incorporate the CDR(s) noncovalently. The CDRs permit the antigen binding protein to specifically bind to a particular antigen of interest.

The assignment of amino acids to each domain is in accordance with the definitions of Kabat et al. in Sequences of Proteins of Immunological Interest, 5th Ed., US Dept. of Health and Human Services, PHS, NIH, NIH Publication no. 91-3242, 1991 (“Kabat numbering”). Other numbering systems for the amino acids in immunoglobulin chains include IMGT® (international ImMunoGeneTics information system; Lefranc et al, Dev. Comp. Immunol. 29:185-203; 2005) and AHo (Honegger and Pluckthun, J. Mol. Biol. 309(3):657-670; 2001); Chothia (Al-Lazikani et al., 1997 Journal of Molecular Biology 273:927-948; Contact (Maccallum et al., 1996 Journal of Molecular Biology 262:732-745, and Aho (Honegger and Pluckthun 2001 Journal of Molecular Biology 309:657-670.

An “antibody” and “antibodies” and related terms used herein refers to an intact immunoglobulin or to an antigen binding portion thereof that binds specifically to an antigen. Antigen binding portions may be produced by recombinant DNA techniques or by enzymatic or chemical cleavage of intact antibodies. Antigen binding portions include, inter alia, Fab, Fab′, F(ab′)₂, Fv, domain antibodies (dAbs), and complementarity determining region (CDR) fragments, single-chain antibodies (scFvs), chimeric antibodies, diabodies, triabodies, tetrabodies, nanobodies, and polypeptides that contain at least a portion of an immunoglobulin that is sufficient to confer specific antigen binding to the polypeptide.

Antibodies include recombinantly produced antibodies and antigen binding portions. Antibodies include non-human, chimeric, humanized and fully human antibodies. Antibodies include monospecific, multispecific (e.g., bispecific, trispecific and higher order specificities). Antibodies include tetrameric antibodies, light chain monomers, heavy chain monomers, light chain dimers, heavy chain dimers. Antibodies include F(ab′)₂ fragments, Fab′ fragments and Fab fragments. Antibodies include single domain antibodies (nanobodies), monovalent antibodies, single chain antibodies, single chain variable fragment (“single chain”) antibodies (also referred to as scFv), camelized antibodies, affibodies, disulfide-linked Fvs (sdFv), anti-idiotypic antibodies (anti-Id), and minibodies. Antibodies include monoclonal and polyclonal populations.

An “antigen binding domain,” “antigen binding region,” or “antigen binding site” and other related terms used herein refer to a portion of an antigen binding protein that contains amino acid residues (or other moieties) that interact with an antigen and contribute to the antigen binding protein's specificity and affinity for the antigen. For an antibody that specifically binds to its antigen, this will include at least part of at least one of its CDR domains.

The terms “specific binding”, “specifically binds” or “specifically binding” and other related terms, as used herein in the context of an antibody or antigen binding protein or antibody fragment, refer to non-covalent or covalent preferential binding to an antigen relative to other molecules or moieties (e.g., an antibody specifically binds to a particular antigen relative to other available antigens). In one embodiment, an antibody specifically binds to a target antigen if it binds to the antigen with a dissociation constant K_{D} of 10⁻⁵ M or less, or 10⁻⁶ M or less, or 10⁻⁷ M or less, or 10⁻⁸ M or less, or 10⁻⁹ M or less, or 10⁻¹⁰ M or less, or 10⁻¹¹ M or less.

In one embodiment, binding specificity of an antibody or antigen binding protein or antibody fragment can be measure by ELISA, radioimmune assay (RIA), enzyme immune assay (EIA), electrochemiluminescence assays (ECL), immunoradiometric assay (IRMA), or surface plasmon resonance (SPR) assay.

For example, in various embodiments a dissociation constant (K_{D}) can be measured using a BIACORE surface plasmon resonance assay. Surface plasmon resonance (SPR) refers to an optical phenomenon that allows for the analysis of real-time interactions by detection of alterations in protein concentrations within a biosensor matrix, for example using the BIACORE system (Biacore Life Sciences division of GE Healthcare, Piscataway, NJ).

An “epitope” and related terms as used herein refers to a portion of an antigen that is bound by an antigen binding protein (e.g., by an antibody or an antigen binding portion thereof). An epitope can comprise portions of two or more antigens that are bound by an antigen binding protein. An epitope can comprise non-contiguous portions of an antigen or of two or more antigens (e.g., amino acid residues that are not contiguous in an antigen's primary sequence but that, in the context of the antigen's tertiary and quaternary structure, are near enough to each other to be bound by an antigen binding protein). Generally, the variable regions, particularly the CDRs, of an antibody interact with the epitope. Unless otherwise indicated, the term “antibody” includes, in addition to antibodies comprising full-length heavy chains and full-length light chains, derivatives, variants, fragments, and muteins thereof, examples of which are described below.

An “antibody fragment”, “antibody portion”, “antigen-binding fragment of an antibody”, or “antigen-binding portion of an antibody” and other related terms used herein refer to a molecule other than an intact antibody that comprises a portion of an intact antibody that binds the antigen to which the intact antibody binds. Examples of antibody fragments include, but are not limited to, single-chain antibody molecules (e.g. scFvs), Fv, Fab, Fab′, Fab′-SH, F(ab′)₂; Fd; and Fv fragments, as well as dAb; diabodies; linear antibodies; polypeptides that contain at least a portion of an antibody that is sufficient to confer specific antigen binding to the polypeptide. Antigen binding portions of an antibody may be produced by recombinant DNA techniques or by enzymatic or chemical cleavage of intact antibodies. Antigen binding portions include, inter alia, scFvs, Fab, Fab′, F(ab′)₂, Fv, domain antibodies (dAbs), and complementarity determining region (CDR) fragments, chimeric antibodies, diabodies, triabodies, tetrabodies, and polypeptides that contain at least a portion of an immunoglobulin that is sufficient to confer antigen binding properties to the antibody fragment. In one embodiment, dimeric antigen receptors comprising a Fab fragment joined to a hinge, transmembrane and intracellular regions are described herein.

The terms “Fab”, “Fab fragment” and other related terms refers to a monovalent fragment comprising a variable light chain region (V_{L}), constant light chain region (C_{L}), variable heavy chain region (V_{H}), and first constant region (C_{H1}). A Fab is capable of binding an antigen. An F(ab′)₂ fragment is a bivalent fragment comprising two Fab fragments linked by a disulfide bridge at the hinge region. A F(Ab′)₂ has antigen binding capability. An Fd fragment comprises V_{H} and C_{H1} regions. An Fv fragment comprises V_{L} and V_{H} regions. An Fv can bind an antigen. A dAb fragment has a V_{H} domain, a V_{L} domain, or an antigen-binding fragment of a V_{H} or VL domain (U.S. Pat. Nos. 6,846,634 and 6,696,245; U.S. published Application Nos. 2002/02512, 2004/0202995, 2004/0038291, 2004/0009507, 2003/0039958; and Ward et al., Nature 341:544-546, 1989).

The term “human antibody” refers to antibodies that have one or more variable and constant regions derived from human immunoglobulin sequences. In one embodiment, all of the variable and constant domains of a human antibody are derived from human immunoglobulin sequences (e.g., a fully human antibody). These antibodies may be prepared in a variety of ways, examples of which are described below, including through recombinant methodologies or through immunization with an antigen of interest of a mouse that is genetically modified to express antibodies derived from human heavy and/or light chain-encoding genes.

A “humanized” antibody refers to an antibody having a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies may be found in U.S. Pat. Nos. 6,054,297, 5,886,152 and 5,877,293.

The term “chimeric antibody” and related terms used herein refers to an antibody that contains one or more regions from a first antibody and one or more regions from one or more other antibodies. In one embodiment, one or more of the CDRs are derived from a human antibody. In another embodiment, all of the CDRs are derived from a human antibody. In another embodiment, the CDRs from more than one human antibody are mixed and matched in a chimeric antibody. For instance, a chimeric antibody may comprise a CDR1 from the light chain of a first human antibody, a CDR2 and a CDR3 from the light chain of a second human antibody, and the CDRs from the heavy chain from a third antibody. In another example, the CDRs originate from different species such as human and mouse, or human and rabbit, or human and goat. One skilled in the art will appreciate that other combinations are possible.

Further, the framework regions may be derived from one of the same antibodies, from one or more different antibodies, such as a human antibody, or from a humanized antibody. In one example of a chimeric antibody, a portion of the heavy and/or light chain is identical with, homologous to, or derived from an antibody from a particular species or belonging to a particular antibody class or subclass, while the remainder of the chain(s) is/are identical with, homologous to, or derived from an antibody (-ies) from another species or belonging to another antibody class or subclass. Also included are fragments of such antibodies that exhibit the desired biological activity (i.e., the ability to specifically bind a target antigen).

As used herein, the term “variant” polypeptides and “variants” of polypeptides refers to a polypeptide comprising an amino acid sequence with one or more amino acid residues inserted into, deleted from and/or substituted into the amino acid sequence relative to a reference polypeptide sequence. Polypeptide variants include fusion proteins. In the same manner, a variant polynucleotide comprises a nucleotide sequence with one or more nucleotides inserted into, deleted from and/or substituted into the nucleotide sequence relative to another polynucleotide sequence. Polynucleotide variants include fusion polynucleotides.

As used herein, the term “derivative” of a polypeptide is a polypeptide (e.g., an antibody) that has been chemically modified, e.g., via phosphorylation, glycosylation, or conjugation to another chemical moiety such as, for example, polyethylene glycol, albumin (e.g., human serum albumin).

The term “hinge” refers to an amino acid segment that is generally found between two domains of a protein and may allow for flexibility of the overall construct and movement of one or both of the domains relative to one another. Structurally, a hinge region comprises from about 10 to about 100 amino acids, e.g., from about 15 to about 75 amino acids, from about 20 to about 50 amino acids, or from about 30 to about 60 amino acids. In some embodiments, a hinge region in a polypeptide is about 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 amino acids in length. A hinge region can be derived from a hinge region of a naturally-occurring protein, such as a CD8 hinge region or a fragment thereof, a CD28 hinge region, or a fragment thereof, or a hinge region of an antibody (e.g., IgG, IgA, IgM, IgE, or IgD antibodies), such as a hinge region that joins the constant domains CH1 and CH2 of an antibody. A hinge region used in an engineered polypeptide can incorporate hinge regions (or fragments thereof) from more than one naturally-occurring protein. The hinge region can be derived from an antibody and may or may not comprise one or more constant regions of the antibody, or the hinge region comprises the hinge region of an antibody and the CH3 constant region of the antibody, or the hinge region comprises the hinge region of an antibody and the CH2 and CH3 constant regions of the antibody, or the hinge region is a non-naturally occurring peptide, or the hinge region is disposed between the C-terminus of the scFv and the N-terminus of the transmembrane domain. In some embodiments, the hinge region comprises any one or any combination of two or more regions comprising an upper, core or lower hinge sequences from an IgG1, IgG2, IgG3 or IgG4 immunoglobulin molecule. In some embodiments, the hinge region comprises one, two, three or more cysteines that can form at least one, two, three or more interchain disulfide bonds.

The term “Fc” or “Fc region” as used herein refers to the portion of an antibody heavy chain constant region beginning in or after the hinge region and ending at the C-terminus of the heavy chain. The Fc region comprises at least a portion of the CH2 and CH3 regions, and may or may not include a portion of the hinge region. An Fc region can bind Fc cell surface receptors and some proteins of the immune complement system. An Fc region exhibits effector function, including any one or any combination of two or more activities including complement-dependent cytotoxicity (CDC), antibody-dependent cell-mediated cytotoxicity (ADCC), antibody-dependent phagocytosis (ADP), opsonization and/or cell binding.

The term “labeled” or related terms as used herein with respect to a polypeptide refers to joinder thereof to a detectable label or moiety for detection. Exemplary detectable labels or moieties include radioactive, colorimetric, antigenic, or enzymatic labels/moieties, a detectable bead (such as a magnetic or electrodense (e.g., gold) bead), biotin, streptavidin or protein A. A variety of labels can be employed, including, but not limited to, radionuclides, fluorescers, enzymes, enzyme substrates, enzyme cofactors, enzyme inhibitors and ligands (e.g., biotin, haptens).

The term “Chimeric Antigen Receptor” or “CAR” refers to a single chain fusion protein comprising an extracellular antigen-binding protein that is fused to an intracellular domain. The CAR extracellular antigen-binding domain can be a single chain variable fragment (scFv or sFv) derived from fusing the variable heavy and light regions of a monoclonal antibody, such as a human monoclonal antibody. In one embodiment, a CAR comprises (i) an antigen binding protein comprising a heavy chain variable (VH) domain and a light chain variable (VL) domain wherein the VH and VL domains are joined together by a peptide linker; (ii) a hinge domain, (iii) a transmembrane domain; and (iv) an intracellular domain comprising an intracellular signaling sequence.

As used herein, “tumor specific antigen” refers to an antigen expressed by a tumor cell and not expressed by normal cells. Tumor specific antigens may be, for example, antigens resulting from the production of a variant protein due to mutation (including point mutations, translocations, viral insertions, as nonlimiting examples) or aberrant splicing, for example.

A “tumor associated antigen” refers to an antigen expressed by a tumor cell that is also expressed by some normal cells. Tumor specific antigens may be overexpressed on tumor cells with respect to the level of expression on normal cells of the same type.

A “checkpoint protein” or “checkpoint molecule” is a cell surface molecule that acts as a negative regulator of immune responses. Checkpoint proteins can be expressed on the surface of tumor cells, leading to downregulation of immune responses by T cells. Nonlimiting examples of checkpoint proteins include PD-L1 and PD-L2.

A “vector” and related terms used herein refers to a nucleic acid molecule (e.g., DNA or RNA) which can be operably linked to foreign genetic material (e.g., nucleic acid transgene) and include at least one of: one or more recombination sequences (which may be, for example, homology arms for insertion of adjacent sequences into another nucleic acid molecule), one or more origins of replication or autonomous replication sequences, and one or more selectable or detectable markers. Vectors can be used as a vehicle to introduce foreign genetic material into a cell (e.g., a host cell). Vectors can include at least one restriction endonuclease recognition sequence for insertion of the transgene into the vector and/or can include recombination sites for recombinational cloning of a nucleic acid sequence or gene into the vector. Vectors can include one or more promoters that can be used to express a gene inserted into the vector in a host cell of interest. Vectors can include at least one gene sequence that confers antibiotic resistance or a selectable characteristic to aid in selection of host cells that harbor a vector-transgene construct. Vectors can be single-stranded or double-stranded nucleic acid molecules and can be linear or circular nucleic acid molecules. One type of vector is a “plasmid,” which refers to a linear or circular double stranded extrachromosomal DNA molecule which can be linked to a transgene, and is capable of replicating in a host cell, and may be configured for transcribing the transgene (for example, includes a promoter and optionally other gene regulatory sequences for expression of an operably linked transgene). A viral vector typically contains viral RNA or DNA backbone sequences which can be linked to the transgene. The viral backbone sequences can be modified to disable infection but retain insertion of the viral backbone and the co-linked transgene into a host cell genome. Examples of viral vectors include retroviral, lentiviral, adenoviral, adeno-associated viral, baculoviral, papovaviral, vaccinia viral, herpes simplex viral and Epstein Barr viral vectors. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome.

An “expression vector” is a type of vector that can contain one or more regulatory sequences, such as inducible and/or constitutive promoters and enhancers. Expression vectors can include ribosomal binding sites and/or polyadenylation sites. Expression vectors can optionally include one or more origin of replication sequence. Regulatory sequences direct transcription, or transcription and translation, of a transgene linked to the expression vector which is transduced into a host cell. The regulatory sequence(s) can control the level, timing and/or location of expression of the transgene. The regulatory sequence can, for example, exert its effects directly on the transgene, or through the action of one or more other molecules (e.g., polypeptides that bind to the regulatory sequence and/or the nucleic acid). Regulatory sequences can be part of a vector. Further examples of regulatory sequences are described in, for example, Goeddel, 1990, Gene Expression Technology: Methods in Enzymology 185, Academic Press, San Diego, Calif. And Baron et al., 1995, Nucleic Acids Res. 23:3605-3606. An expression vector can comprise nucleic acids that encode at least a portion of any of the dimeric antigen receptors (DAR) or antigen-binding portions thereof that are described herein.

A transgene is “operably linked” to a promoter when there is linkage between the transgene and the promoter to permit expression of the transgene sequences.

The terms “transfected” or “transformed” or “transduced” or other related terms used herein refer to a process by which exogenous nucleic acid (e.g., transgene) is transferred or introduced into a host cell. A “transfected” or “transformed” or “transduced” host cell is one into which an exogenous nucleic acid (for example, including a transgene) has been introduced. “Transduced” is typically used to indicate gene transfer by means of a virus (e.g., a retrovirus or lentivirus). The term host cell includes the primary subject cell and its progeny. Exogenous nucleic acids encoding at least a portion of any of the engineered scFv-TCRs or subunits thereof, such as any of the engineered TCR polypeptides e.g., scFv-TCR subunit polypeptides and N-terminally truncated TCR subunit polypeptides that are described herein can be introduced into a host cell. For example, expression vectors or DNA fragments comprising at least a portion of any of the scFv-TCRs or subunits thereof that are described herein can be introduced into a host cell, and the host cell can express polypeptides comprising at least a portion of the scFv-TCR or a subunit thereof, e.g., an scFv-TCR subunit polypeptide or N-terminally truncated TCR polypeptide, that are described herein.

In various embodiments, a host cell can be transfected or transduced with at least one expression vector or nucleic acid fragment in which a promoter is operably linked to a nucleic acid sequence encoding an scFv-TCR polypeptide to generate a transfected/transformed host cell that can be cultured under conditions suitable for expression of the scFv-TCR polypeptide by the transfected/transformed host cell.

Typically, a host cell is a cultured cell that can be transformed or transfected with a polypeptide-encoding nucleic acid, which can then be expressed in the host cell. The phrase “transgenic host cell” or “recombinant host cell” can be used to denote a host cell that has been transduced, transformed, or transfected with a nucleic acid to be expressed. A host cell also can be a cell that comprises the nucleic acid but does not express it at a desired level unless a regulatory sequence is introduced into the host cell such that it becomes operably linked with the nucleic acid. It is understood that the term host cell refers not only to the particular subject cell but also to the progeny or potential progeny of such a cell. Because certain modifications may occur in succeeding generations due to, e.g., mutation or environmental influence, such progeny may not, in fact, be identical to the parent cell, but are still included within the scope of the term as used herein. A host cell, or a population of host cells, harboring a vector (e.g., an expression vector) operably linked to at least one nucleic acid encoding one or more of the scFv-TCR polypeptides that are described herein.

A foreign nucleic acid introduced into cells can comprise an expression vector having a promoter operably linked to a transgene, and the host cell can be used to express the nucleic acid and/or polypeptide encoded by the foreign nucleic acid (transgene). A host cell (or a population thereof) can be a cultured cell or can be extracted from a subject. A cultured cell can be a cell of a cell line or a primary cell. The host cell (or a population thereof) includes the primary subject cell and its progeny without regard for the number of passages. The host cell (or a population thereof) includes immortalized cell lines. Host cells encompass progeny cells. Progeny cells may or may not harbor identical genetic material compared to the parent cell. In one embodiment, a host cell describes any cell (including its progeny) that has been modified, transfected, transduced, transformed, and/or manipulated in any way to express an engineered TCR polypeptide or scFv-TCR as disclosed herein. In one example, the host cell (or population thereof) can be transfected or transduced with an expression vector that includes a nucleic acid encoding the polypeptide(s) described herein. Host cells and populations thereof can harbor an expression cassette or expression vector, including a retroviral or lentiviral vector or portion thereof, that is stably integrated into the host's genome, or can harbor an extrachromosomal expression vector. In various embodiments, host cells and populations thereof can harbor one or more expression cassettes that include transgenes for expressing the first and second engineered TCR polypeptides of a scFv-TCR that are integrated into the host genome.

The terms “host cell” or “population of host cells” or related terms as used herein refer to a cell (or a population of cells) into which foreign (exogenous or transgene) nucleic acids have been introduced. The term “population of host cells” can refer to a population of cells, particularly primary cells, that has been transfected or transduced with an exogenous nucleic acid sequence encoding, for example, an scFv-TCR polypeptide, where scFv-TCR polypeptide-expressing cells may represent less than 100% of the population. For example, a population of host cells transfected with at least one nucleic acid molecule that encodes an scFv-TCR polypeptide may comprise at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, cells that express the scFv-TCR. The percentage of cells of the host cell population that expresses a gene of interest, such as a gene encoding an engineered TCR subunit, can optionally be increased, for example, by cell-sorting flow cytometry, selective capture of scFv-TCR-positive cells, or by expansion on target cells or molecules (e.g., cells expressing an antigen specifically bound by the scFv-TCR polypeptide).

Engineered polypeptides of the present disclosure (e.g., scFv-TCR polypeptides and/or truncated TCR polypeptides, which may be incorporated into ScFvTCRs) can be produced using any method known in the art. In one example, the polypeptides are produced by recombinant nucleic acid methods by inserting a nucleic acid sequence (e.g., DNA) encoding the polypeptide into a recombinant expression vector which is introduced into a host cell and expressed by the host cell under conditions promoting expression.

General techniques for recombinant nucleic acid manipulations are described for example in Sambrook et al., in Molecular Cloning: A Laboratory Manual, Vols. 1-3, Cold Spring Harbor Laboratory Press, 2 ed., 1989, or F. Ausubel et al., in Current Protocols in Molecular Biology (Green Publishing and Wiley-Interscience: New York, 1987) and periodic updates, herein incorporated by reference in their entireties. The nucleic acid (e.g., DNA) encoding the polypeptide is operably linked to an expression vector carrying one or more suitable transcriptional or translational regulatory elements derived from mammalian, viral, or insect genes. Such regulatory elements include a transcriptional promoter, an optional operator sequence to control transcription, a sequence encoding suitable mRNA ribosomal binding sites, and sequences that control the termination of transcription and translation. The expression vector can include an origin or replication that confers replication capabilities in the host cell. The expression vector can include a gene that confers selection to facilitate recognition of transgenic host cells (e.g., transformants).

The recombinant DNA can also encode any type of protein tag sequence that may be useful for purifying the protein. Examples of protein tags include but are not limited to a histidine tag, a FLAG tag, a myc tag, an HA tag, or a GST tag. Appropriate cloning and expression vectors for use with bacterial, fungal, yeast, and mammalian cellular hosts can be found in Cloning Vectors: A Laboratory Manual, (Elsevier, N.Y., 1985).

The expression vector construct can be introduced into the host cell using a method appropriate for the host cell. A variety of methods for introducing nucleic acids into host cells are known in the art, including, but not limited to, electroporation; transfection employing calcium chloride, rubidium chloride, calcium phosphate, DEAE-dextran, or other substances; viral transfection; non-viral transfection; microprojectile bombardment; lipofection; and infection (e.g., where the vector is an infectious agent). Suitable host cells include prokaryotes, yeast, mammalian cells, or bacterial cells.

A host cell can be a prokaryote, for example, E. coli, or it can be a eukaryote, for example, a single-celled eukaryote (e.g., a yeast or other fungus), a plant cell (e.g., a tobacco or tomato plant cell), a mammalian cell (e.g., a human cell, a monkey cell, a hamster cell, a rat cell, a mouse cell, or an insect cell) or a hybridoma. In various embodiments, host cells comprise non-human cells including CHO, BHK, NS0, SP2/0, and YB2/0. In other embodiments, host cells comprise human cells including HEK293, HT-1080, Huh-7 and PER.C6. In some embodiments, a host cell is a mammalian host cell, but is not a human host cell. Examples of host cells include the COS-7 line of monkey kidney cells (ATCC CRL 1651) (see Gluzman et al., 1981, Cell 23:175), L cells, C127 cells, 3T3 cells (ATCC CCL 163), Chinese hamster ovary (CHO) cells or their derivatives such as Veggie CHO and related cell lines which grow in serum-free media (see Rasmussen et al., 1998, Cytotechnology 28:31) or CHO strain DX-B 11, which is deficient in DHFR (see Urlaub et al., 1980, Proc. Natl. Acad. Sci. USA 77:4216-20), HeLa cells, BHK (ATCC CRL 10) cell lines, the CV1/EBNA cell line derived from the African green monkey kidney cell line CV1 (ATCC CCL 70) (see McMahan et al., 1991, EMBO J. 10:2821), human embryonic kidney cells such as 293, 293 EBNA or MSR 293, human epidermal A431 cells, human Colo 205 cells, other transformed primate cell lines, normal diploid cells, cell strains derived from in vitro culture of primary tissue, primary explants, HL-60, U937, HaK or Jurkat cells. In one embodiment, host cells include lymphoid cells such as Y0, NS0 or Sp20.

In various embodiments disclosed herein, host cells are immunological cells such as T lymphocytes (e.g., T cells, regulatory T cells, gamma-delta T cells, and cytotoxic T cells), NK (natural killer) cells, macrophages, dendritic cells, mast cells, eosinophils, B lymphocytes, or monocytes. In some embodiments, the NK cells comprise cord blood-derived NK cells, or placental derived NK cells. In various embodiments a population of host cells can comprise human T lymphocytes or human NK cells, for example, can be primary human T cells or primary human NK cells.

Nucleic acids encoding any of the various polypeptides disclosed herein may be synthesized chemically. Codon usage may be selected so as to improve expression in a cell. Such codon usage will depend on the cell type selected. Specialized codon usage patterns have been developed for E. coli and other bacteria, as well as mammalian cells, plant cells, yeast cells and insect cells. See for example: Mayfield et al., Proc. Natl. Acad. Sci. USA. 2003 100(2):438-42; Sinclair et al. Protein Expr. Purif. 2002 (1):96-105; Connell N D. Curr. Opin. Biotechnol. 2001 12(5):446-9; Makrides et al. Microbiol. Rev. 1996 60(3):512-38; and Sharp et al. Yeast. 1991 7(7):657-78.

In certain embodiments, the polypeptides described herein (e.g., scFv-TCR polypeptides) can further comprise post-translational modifications. Exemplary post-translational protein modifications include phosphorylation, acetylation, methylation, ADP-ribosylation, ubiquitination, glycosylation, afucosylation, carbonylation, sumoylation, biotinylation or addition of a polypeptide side chain or of a hydrophobic group. As a result, the polypeptides may contain non-amino acid elements, such as lipids, poly- or mono-saccharide, and phosphates.

The present disclosure provides therapeutic compositions comprising any of the cells or cell populations described herein that express a scFv-TCR as described herein in an admixture with a pharmaceutically-acceptable excipient. Excipients encompass, for example, physiologically-compatible and osmotically balanced buffers such as PBS, HBSS, Ringer's solution or Tyrode's solution, or variations thereof. A cryoprotectant, such as but not limited to glycerol, DMSO, an alcohol, a sugar alcohol, or a polyol, can be included in the composition. In addition, a pharmaceutical composition can include buffering agents, stabilizing agents, preservatives, non-ionic detergents, anti-oxidants and isotonifiers. Carriers, stabilizers, diluents or fillers (e.g., sucrose and sorbitol), lubricating agents, glidants, and anti-adhesives (e.g., magnesium stearate, zinc stearate, stearic acid, silicas, hydrogenated vegetable oils, or talc) may be included. The pharmaceutically-acceptable excipients will be chosen so as not to interfere with the viability or activity of the cells.

Therapeutic compositions and methods for preparing them are well known in the art and are found, for example, in “Remington: The Science and Practice of Pharmacy” (20^{th} ed., ed. A. R. Gennaro A R., 2000, Lippincott Williams & Wilkins, Philadelphia, Pa.). Therapeutic compositions can be formulated for parenteral administration may, and can for example, contain excipients, sterile water, saline, polyalkylene glycols such as polyethylene glycol, oils of vegetable origin, or hydrogenated napthalenes. A pharmaceutical composition for injection or infusion, including injection or infusion of cells, can comprise, for example, PBS, HBSS, Ringer's solution, Tyrode's solution, or a related solution, for example having one or more substituted, added, or removed components. Biocompatible, biodegradable lactide polymer, lactide/glycolide copolymer, or polyoxyethylene-polyoxypropylene copolymers or nanoparticulate formulations (e.g., biodegradable nanoparticles, solid lipid nanoparticles, liposomes) may be included. Other potentially useful parenteral delivery systems include ethylene-vinyl acetate copolymer particles, osmotic pumps, implantable infusion systems, and liposomes. The concentration of cells in the formulation varies depending upon a number of factors, including the body weight of the subject, stage or size of the tumor being treated, and the route of administration.

Addition salts that may be used in a formulation include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid phosphoric acid, or the like. Metal complexes that may be in a pharmaceutical formulation include zinc, iron, and the like.

The term “subject” as used herein refers to human and non-human animals, including vertebrates, mammals and non-mammals. In one embodiment, the subject can be human, non-human primates, simian, ape, murine (e.g., mice and rats), bovine, porcine, equine, canine, feline, caprine, lupine, ranine or piscine.

The term “administering”, “administered” and grammatical variants refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In one embodiment, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods. Cells expressing any of the scFv-TCRs or engineered polypeptides thereof described herein can be administered to a subject using art-known methods and delivery routes.

The terms “effective amount”, “therapeutically effective amount” or “effective dose” or related terms may be used interchangeably and for scFv-TCR-expressing cells refer the number of cells expressing an scFv-TCR, e.g., scFv-TCR-T cells, as described herein that when administered to a subject, is sufficient to effect a measurable improvement or prevention of a disease or disorder associated with tumor or cancer antigen expression. Therapeutically effective amounts of scFv-TCR-expressing cells as provided herein, when used alone or in combination, will vary depending upon the relative effectiveness of the cells (e.g., in inhibiting tumor growth) and depending upon the subject and disease condition being treated, the weight and age and sex of the subject, the severity of the disease condition in the subject, the manner of administration and the like, which can readily be determined by one of ordinary skill in the art.

For example, a therapeutically effective amount can comprise a dose of about 10³-10¹² transgenic host cells, such as between about 10⁴ and about 10¹¹ cells, or between about 10⁵ and about 10¹⁰ cells administered to the subject. The transgenic host cells can harbor one or more nucleic acids that encode the engineered TCR polypeptide subunits of any of the scFv-TCRs described herein. The therapeutically effective amount can be determined by considering the subject to receive the therapeutically effective amount and the disease/disorder to be treated which may be ascertained by one skilled in the art using known techniques. The therapeutically effective amount may consider factors pertaining to the subject such as age, body weight, general health, sex, diet, time of administration, drug interaction, and the severity of the disease/disorder. The therapeutically effective amount may consider the purity of the transgenic host cells and the percentage of scFv-TCR-expressing cells within the population, which can be about 8%-98% or higher levels of purity. The therapeutically effective amount of the transgenic host cells can be administered to the subject at least once, or twice, three times, 4 times, 5 times, or more over a period of time. The period of time can be per day, per week, per month, or per year. The therapeutically effective amount of the transgenic cells administered to the subject can be the same each time or can be increased or decreased at each administration event. In some embodiments, the therapeutically effective amount of the transgenic cells may be administered to the subject until the tumor size or number of cancer cells is reduced by 5%-90% or more, compared to the tumor size or number of cancer cells prior to administration of the transgenic host cells.

The present disclosure provides methods for treating a subject having a disease/disorder associated with expression or over-expression of one or more tumor-associated antigens. The disease comprises cancer or tumor cells expressing the tumor-associated antigens, or checkpoint proteins, for example, PD-L1. In various embodiments, the cancer or tumor includes cancer of the prostate, breast, ovary, head and neck, bladder, skin, anus, rectum, pancreas, lung (including non-small cell lung and small cell lung cancers), bone, leiomyoma, brain (including glioma and glioblastoma), esophagus, liver, kidney, stomach, colon, cervix, uterus, endometrium, vulva, larynx, vagina, bone, nasal cavity, paranasal sinus, nasopharynx, oral cavity, oropharynx, hypolarynx, salivary glands, ureter, urethra, penis and testis. In further embodiments, the cancer comprises a hematological cancer, including leukemias, lymphomas, myelomas, and B cell lymphomas.

### Single Chain Antibody Gamma Delta T Cell Receptors (ScFv γδTCRs) and Polypeptides

Engineered scFv T cell receptors (scFv-TCRs) are provided that comprise a polypeptide that combines a targeting single chain antibody (scFv) with domains of a native T cell receptor subunit. The term “T cell receptor,” or “TCR,” refers to a heterodimeric receptor composed of αβ or γδ chains that pair on the surface of a T cell. Each α, β, γ, and δ chain is composed of two Ig-like domains: a variable domain (V) that confers antigen recognition through the complementarity determining regions (CDR), followed by a constant domain that includes a constant extracellular domain that includes a connecting peptide, and a transmembrane (TM) region. The TM regions of TCR chains associate with the invariant subunits of the CD3 signaling apparatus, forming a TCR complex.

TCR chains include an extracellular region that includes a variable immunoglobulin-like (V) domain, a constant immunoglobulin-like (C) domain, and a connecting peptide. The connecting peptide is followed by a transmembrane domain and then a short intracellular domain. The N-terminal variable domains of the core TCR subunits (α, β, γ, and delta), which are unique to each gene product, form the antigen-binding domain of the TCR. Following the N-terminal variable domain, each TCR core subunit polypeptide has an extracellular constant domain that has the same sequence for each type of polypeptide chain (α, β, γ, and δ) and includes an immunoglobulin-like domain followed by a region (the “connecting peptide”) closest to the membrane and having residues for interchain binding via one or more disulfide bonds. The extracellular constant region is followed by a transmembrane domain, and then, for the TCRβ and TCRγ subunits, by a short intracellular domain. The TCRα chain also has two amino acids (e.g., “Ser-Ser”) C-terminal to the transmembrane domain that may be intracellular or associated with the inner leaflet of the cell membrane, and TCRδ has a C-terminal amino acid (Leu) that may extend beyond a strictly defined transmembrane domain but are considered part of the transmembrane domain in the description herein. The extracellular Ig-like C domain, connecting peptide, transmembrane domain, and, for TCRβ and TCRγ subunits, intracellular domain are together referred to as the constant region (or complete constant region) of the TCR subunits, and the extracellular Ig-like C domain and connecting peptide together may be referred to as the extracellular constant region. Assembly of the TCR complex occurs in the endoplasmic reticulum and Golgi apparatus. While dimers of the TCRα and TCRβ. TCRγ and TCRδ, CD3δε, and CD3γε associate with one another largely via their extracellular domains, CD3ζ dimers are bound via intermembrane noncovalent linkages. Core complex TCR chains assemble with CD3 dimers largely through intramembrane interactions relying on acidic transmembrane residues in the CD3 polypeptides and basic residues in the TCR core polypeptide transmembrane domains. The failure of core polypeptides or CD3 dimer polypeptides to assemble in a TCR complex results in exposed charged residues that serve as a signal for degradation of the unassembled subunits (Wucherpfennig et al. (2009) Cold Spring Harb Perspect Biol 2:a005140).

The engineered scFv-TCRs provided herein comprise a polypeptide that combines an scFv with a portion of a TCR subunit that includes at least a portion of a connecting peptide, a transmembrane domain, and optionally an intracellular domain, of a native TCR subunit. The scFv-TCR polypeptides are chimeric, where the scFv, which can specifically bind a tumor associated antigen or a checkpoint protein, for example, substitutes for the variable extracellular domain of a native TCR. In various embodiments the chimeric scFv-TCR polypeptides provided herein include domains of TCRγδ subunits or sequences having homology thereto, for example, an scFv-TCR polypeptide may be an scFv-TCRδ polypeptide or an scFv-TCRγ polypeptide.

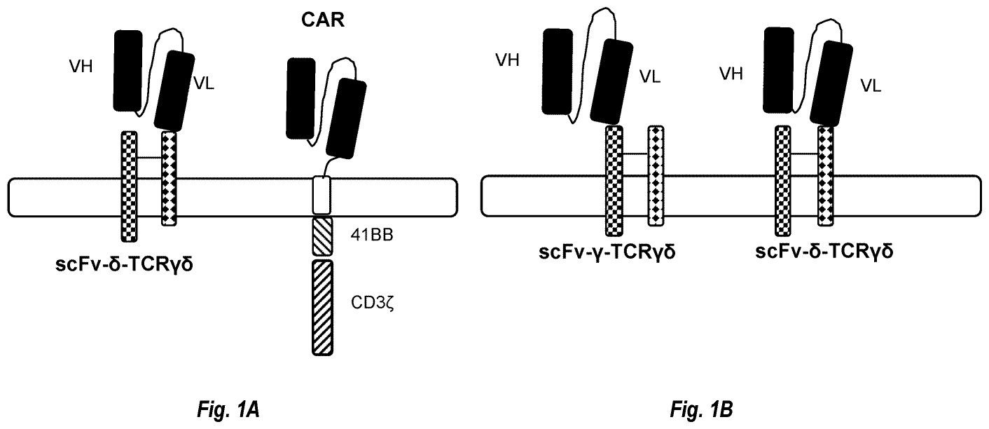

In some embodiments, a chimeric scFv-TCR polypeptide and an engineered N-terminally truncated TCR polypeptide are produced by an engineered host cell. For example, an engineered host cell may include a nucleic acid sequence encoding an scFv-TCRδ polypeptide and a nucleic acid sequence encoding an N-terminally truncated TCRγ polypeptide (NT-TCRγ), where the host cell expresses an scFv-δ-TCRγ (TCR having a first polypeptide in which the scFv substitutes for the variable domain of the TCRδ chain and a second polypeptide that comprises an N-terminally truncated TCRγ chain lacking an extracellular variable domain). In another example, an engineered host cell may include a nucleic acid sequence encoding an scFv-TCRγ polypeptide and a nucleic acid sequence encoding an N-terminally truncated TCRδ polypeptide (NT-TCRδ), where the host cell expresses an scFv-γ-TCRγδ (TCR having a first polypeptide in which the scFv substitutes for the variable domain of the TCRγ chain and a second polypeptide that comprises an N-terminally truncated TCRδ chain lacking an extracellular variable domain (NT-TCRδ). (See, FIG. 1B).

In some embodiments, a chimeric scFv-TCR polypeptide produced by an engineered host cell may be expressed in the absence of expression of another engineered TCR polypeptide. For example, an scFv-TCRγ may be expressed by a host cell that does not a non-native nucleic acid sequence encoding a second TCR subunit, such as a TCR subunit that includes domains of a TCRδ subunit.

Experiments disclosed herein demonstrate that an engineered scFv-TCRγ subunit, when expressed in T cells in the absence of a partner TCR subunit, i.e., in the absence of expression of a native or engineered TCR subunit, is able to expand and activate in response to target tumor cells and demonstrates cytotoxicity against target tumor cells in vitro and in vivo. In addition, T cells expressing a tumor-targeting scFv-TCRγ secrete less GM-CSF than is secreted by CAR-T cells targeting the same tumor cells, raising the possibility that scFv-TCRγ-T cells might have a better safety profile than analogous CAR-T cells, resulting in fewer occurrences of cytokine release syndrome (See, for example, Sacheva et al. (2019) J. Biol. Chem. 294:5430-37 and U.S. Pat. No. 10,870,730, incorporated herein by reference).

In a first aspect, provided herein is an scFv-TCRγ polypeptide that comprises an scFv fused to a portion of the constant region of a TCRγ subunit or to a polypeptide sequence derived therefrom. In some embodiments, the scFv-TCRγ polypeptide does not comprise a TCRγ variable domain or a complete or substantially complete TCRγ subunit constant region. In some embodiments of this aspect, the scFv-TCRγ subunit comprises, from the N-terminus to C-terminus: (1) an scFv that binds a target antigen; and (2) (i) a combined TCRγ connecting peptide, TCRγ transmembrane domain, and TCRγ intracellular domain sequence; or (2) (ii) a TCRγ connecting peptide, a TCRγ transmembrane domain, and a TCRγ intracellular domain.

In some embodiments, the scFv-TCRγ subunit comprises an scFv that binds a target antigen and a combined TCRγ connecting peptide, TCRγ transmembrane domain, and TCRγ intracellular domain sequence, where the combined TCRγ connecting peptide, TCRγ transmembrane domain, and TCRγ intracellular domain sequence comprises a sequence that has at least 95%, at least 96%, at least 97%, at least 98%, or at least 99% identity to SEQ ID NO: 17. In some embodiments, the combined TCRγ connecting peptide, TCRγ transmembrane domain, and TCRγ intracellular domain sequence comprises the sequence of SEQ ID NO:17. In some embodiments the first engineered TCR subunit consists essentially of an scFv that binds a target antigen and the sequence of SEQ ID NO:17 or an amino acid sequence having at least 95% identity thereto. In some embodiments, the scFv-TCRγ further comprises a signal peptide at the N-terminus thereof.

In some embodiments, the first engineered TCR subunit comprises, from the N-terminus to the C-terminus of the polypeptide: an scFv that binds a target antigen; the connecting peptide of a TCRγ subunit (SEQ ID NO:9) or a peptide having at least 95% identity (for example, at least 95%, at least 96%, at least 97%, at least 98%, or at least 99% identity) thereto; the transmembrane domain of the TCRγ subunit (SEQ ID NO:10) or a transmembrane domain having at least 95% identity (for example, at least 95%, at least 96%, at least 97%, at least 98%, or at least 99% identity) thereto; and the intracellular domain of the TCRγ subunit (SEQ ID NO:11) or an intracellular domain having at least 95% identity (for example, at least 95%, at least 96%, at least 97%, at least 98%, or at least 99% identity) thereto. In some embodiments the first engineered TCR subunit consists essentially of, from the N-terminus to the C-terminus of the polypeptide: an scFv that binds a target antigen; the connecting peptide of a TCRγ subunit (SEQ ID NO:9) or a peptide having at least 95% identity thereto; the transmembrane domain of the TCRγ subunit (SEQ ID NO:10) or a transmembrane domain having at least 95% identity thereto; and the intracellular domain of the TCRγ subunit (SEQ ID NO:11) or an intracellular domain having at least 95% identity thereto. In some embodiments, the scFv-TCRγ further comprises a signal peptide at the N-terminus thereof.

In preferred embodiments of this aspect the first engineered subunit (e.g., the scFv-TCRγ subunit) does not comprise a TCR subunit variable domain (such as a TCRγ variable domain) or a portion thereof. In preferred embodiments the first engineered subunit does not comprise a complete or substantially complete TCR subunit constant region (such as a complete or substantially complete TCRγ constant region). In some preferred embodiments, the extracellular moiety of the first engineered subunit comprises an scFv that specifically binds a target antigen fused to the connecting peptide of a TCRγ subunit (SEQ ID NO:9) or an amino acid sequence having at least 95% identity thereto. In some embodiments, the extracellular moiety of the first engineered subunit consists essentially of an scFv that specifically binds a target antigen fused to the connecting peptide of a TCRγ subunit (SEQ ID NO:9) or an amino acid sequence having at least 95% identity thereto. In some embodiments, the extracellular moiety of the first engineered subunit comprises or consists essentially of an scFv that specifically binds a target antigen connected by a linker of from one to about eighty, from one to about sixty, from one to about forty, from one to about twenty, or from one to about five amino acids to the connecting peptide of a TCRγ subunit (SEQ ID NO:9) or an amino acid sequence having at least 95% identity thereto.

The scFv of the scFv-TCRγ can specifically bind a tumor associated antigen or an immune checkpoint protein. In some embodiments, the scFv-TCRγ comprises an ScFv that specifically binds PD-L1 and comprises the amino acid sequence of SEQ ID NO:25 or an amino acid sequence having at least 95% identity (e.g., at least 96%, at least 97%, at least 98%, or at least 99% identity) to SEQ ID NO:25. In some embodiments, the scFv-TCRγ comprises an ScFv that specifically binds CD19 and comprises the amino acid sequence of SEQ ID NO:60 or an amino acid sequence having at least 95% identity (e.g., at least 96%, at least 97%, at least 98%, or at least 99% identity) to SEQ ID NO:60.

In another aspect, the disclosure provides an ScFv TCRγδ that includes first and second engineered TCR subunits, where the first engineered TCR subunit (scFv-TCRδ) comprises an scFv fused to a portion of the constant region of a TCRδ subunit or to a polypeptide sequence derived therefrom, and the second engineered TCR subunit comprises an N-terminally truncated TCRγ subunit (NT-TCRγ), or a polypeptide sequence derived therefrom. In some embodiments, the scFv-TCRδ subunit does not comprise a TCRδ variable domain or a substantially complete TCRδ subunit constant region. In some embodiments, the NT-TCRγ subunit does not comprise a TCRγ variable domain or a substantially complete TCRγ subunit constant region.

In some embodiments of this aspect, the scFv-TCRδ subunit comprises, from the N-terminus to C-terminus: (1) an scFv that binds a target antigen; and (2) (i) a combined TCRδ connecting peptide and TCRδ transmembrane domain sequence; or (2) (ii) a TCRδ connecting peptide and a TCRδ transmembrane domain.

In some embodiments, the scFv-TCRδ subunit comprises an scFv that binds a target antigen and a combined TCRδ connecting peptide and TCRδ transmembrane domain sequence, where the combined TCRδ connecting peptide and TCRδ transmembrane domain sequence comprises a sequence that has at least 95%, at least 96%, at least 97%, at least 98%, or at least 99% identity to the sequence of SEQ ID NO:27. In some embodiments, the combined TCRδ connecting peptide and TCRδ transmembrane domain sequence comprises the sequence of SEQ ID NO:27. In some embodiments the first engineered TCR subunit (scFv-TCRδ) consists essentially of an scFv that binds a target antigen and the sequence of SEQ ID NO:27 or an amino acid having at least 95% identity thereto. In some embodiments, the scFv-TCRδ further comprises a signal peptide at the N-terminus thereof.

In some embodiments, the first engineered TCR subunit (scFv-TCRδ) comprises, from the N-terminus to C-terminus of the polypeptide: an scFv that binds a target antigen; the connecting peptide of a TCRδ subunit (SEQ ID NO:14) or a peptide having at least at least 95% identity (for example, at least 95%, at least 96%, at least 97%, at least 98%, or at least 99% identity) thereto; and the transmembrane domain of a TCRδ subunit (SEQ ID NO:15) or a transmembrane domain having at least at least 95% identity (for example, at least 95%, at least 96%, at least 97%, at least 98%, or at least 99% identity) thereto. In some embodiments the first engineered TCR subunit consists essentially of, from the N-terminus to the C-terminus of the polypeptide: an scFv that binds a target antigen; the connecting peptide of a TCRδ subunit (SEQ ID NO:14) or a peptide having at least 95% identity thereto and the transmembrane domain of the TCRδ subunit (SEQ ID NO:15) or a transmembrane domain having at least 95% identity thereto. In some embodiments, the scFv-TCRδ further comprises a signal peptide at the N-terminus thereof.

The scFv of the scFv-TCRδ subunit can specifically bind a tumor associated antigen or an immune checkpoint protein. In some embodiments, the scFv-TCRδ subunit comprises an ScFv that specifically binds PD-L1 and comprises the amino acid sequence of SEQ ID NO:25 or an amino acid sequence having at least 95% identity (e.g., at least 96%, at least 97%, at least 98%, or at least 99% identity) to SEQ ID NO:25. In some embodiments, the scFv-TCRδ subunit comprises an ScFv that specifically binds CD19 and comprises the amino acid sequence of SEQ ID NO:60 or an amino acid sequence having at least 95% identity (e.g., at least 96%, at least 97%, at least 98%, or at least 99% identity) to SEQ ID NO:60.

In some embodiments the second engineered TCRγδ subunit can comprise, from the N-terminus to the C-terminus of the polypeptide: a connecting peptide of a TCRγ subunit (SEQ ID NO:9) or a peptide having at least 95% identity (for example, at least 95%, at least 96%, at least 97%, at least 98%, or at least 99% identity) thereto; the transmembrane domain of the TCRγ subunit (SEQ ID NO:10) or a transmembrane domain having at least 95% identity (for example, at least 95%, at least 96%, at least 97%, at least 98%, or at least 99% identity) thereto; and the intracellular domain of the TCRγ subunit (SEQ ID NO:11) or an intracellular domain having at least 95% identity (for example, at least 95%, at least 96%, at least 97%, at least 98%, or at least 99% identity) thereto. In some embodiments the second engineered γδTCR subunit consists essentially of, from the N-terminus to the C-terminus of the polypeptide: the connecting peptide of a TCRγ subunit (SEQ ID NO:9) or a peptide having at least 95% identity thereto; the transmembrane domain of the TCRγ subunit (SEQ ID NO:10) or a transmembrane domain having at least 95% identity thereto; and the intracellular domain of the TCRγ subunit (SEQ ID NO:11) or an intracellular domain having at least 95% identity thereto. In some embodiments, the NT-TCRγ further comprises a signal peptide at the N-terminus thereof.

In some embodiments, the second engineered subunit is an N-terminally truncated TCRγ subunit (NT-TCRγ), which comprises a sequence having at least 95% identity (for example, at least 95%, at least 96%, at least 97%, at least 98%, or at least 99% identity) to SEQ ID NO:17. In some embodiments, the NT-TCRγ subunit, which comprises the combined TCRγ connecting peptide, TCRγ transmembrane domain, and TCRγ intracellular domain sequence comprises the sequence of SEQ ID NO:17. In some embodiments, the NT-TCRγ subunit consists essentially of SEQ ID NO:17. In some embodiments, the NT-TCRγ subunit comprises, from the N-terminus to the C-terminus: (1) a TCRγ connecting peptide comprising the sequence of SEQ ID NO:19; (2) a TCRγ transmembrane domain comprising the sequence of SEQ ID NO:10, and (3) a TCRγ intracellular domain comprising the sequence of SEQ ID NO:11. In some embodiments, the NT-TCRγ subunit further comprises a signal peptide at the N-terminus thereof.

In preferred embodiments of this aspect the first engineered subunit (the scFv-TCRδ subunit) does not comprise a TCR subunit variable domain (such as a TCRδ variable domain) or a portion thereof. In preferred embodiments the first engineered subunit does not comprise a complete or substantially complete TCRδ subunit constant region. In some preferred embodiments, the extracellular moiety of the first engineered subunit comprises an scFv that specifically binds a target antigen fused to the connecting peptide of a TCRδ subunit (SEQ ID NO:14) or an amino acid sequence having at least 95% identity thereto. In some embodiments, the extracellular moiety of the first engineered subunit consists essentially of an scFv that specifically binds a target antigen fused to the connecting peptide of a TCRδ subunit (SEQ ID NO:14) or an amino acid sequence having at least 95% identity thereto.

In preferred embodiments the second engineered TCRγδ subunit (e.g., the NT-TCRγ polypeptide) does not comprise a TCR subunit variable region, e.g., does not include a TCRγ variable region. In preferred embodiments the second engineered TCRγδ subunit does not comprise a complete or substantially complete TCR subunit constant region, e.g., does not include a complete or substantially complete TCRγ subunit constant region. In preferred embodiments the second engineered TCR subunit consists essentially of the connecting peptide of a TCRγ subunit (SEQ ID NO:9) or an amino acid sequence having at least 95% identity thereto, followed by the transmembrane domain of the TCRγ subunit (SEQ ID NO:10) or a transmembrane domain having at least 95% identity thereto, and the intracellular domain of the TCRγ subunit (SEQ ID NO:11) or an intracellular domain having at least 95% identity thereto.

In some exemplary embodiments an ScFv TCRγδ as provided herein includes first and second engineered TCRγδ subunits, where the first engineered TCRγδ subunit is an scFv-TCRδ subunit comprising the amino acid sequence of SEQ ID NO:19 or an amino acid sequence having at least 95% identity (for example, at least 95%, at least 96%, at least 97%, at least 98%, or at least 99% identity) thereto and the second engineered TCRγδ subunit is an NT-TCRγ subunit comprising the amino acid sequence of SEQ ID NO:17 or an amino acid sequence having at least 95% identity (for example, at least 95%, at least 96%, at least 97%, at least 98%, or at least 99% identity) thereto.

In some exemplary embodiments an ScFv TCRγδ as provided herein includes first and second engineered TCRγδ subunits, where the first engineered TCRγδ subunit is an scFv-TCRγ subunit comprising the amino acid sequence of SEQ ID NO:25 or an amino acid sequence having at least 95% identity (for example, at least 95%, at least 96%, at least 97%, at least 98%, or at least 99% identity) thereto and the second engineered TCRγδ subunit is an NT-TCRδ subunit comprising the amino acid sequence of SEQ ID NO:26 or an amino acid sequence having at least 95% identity (for example, at least 95%, at least 96%, at least 97%, at least 98%, or at least 99% identity) thereto.

The engineered scFv-TCRs and ScFv-TCR polypeptides (e.g., scFv-TCRγ polypeptides and scFv-TCRδ polypeptides) as provided herein can be isolated scFv-TCRs and can be situated in the membrane of a host cell, e.g., a host cell engineered to express the TCRs and TCR polypeptides as provided herein. Specifically are engineered scFv-TCRγ polypeptides such as the scFv-TCRγ polypeptides described above, that may not be expressed as components of an engineered multi-subunit TCR.

In various embodiments, any of the scFv-TCR polypeptides described herein may have a peptide linker inserted between one or more domains. Peptide linkers are known in the art, and can range in size from two amino acids to about 100 amino acids, but are more typically between about four and about 50 amino acids in length. For example, a peptide linker can be from about two to about eighty amino acids, from about two to about sixty amino acids, from about two to about forty amino acids, from about two to about thirty amino acids, or from two to about For example, a peptide linker may be included between the extracellular constant sequences derived from a TCR chain and the scFv of an scFv-TCR. A peptide linker can also optionally be included between the extracellular constant sequences derived from a TCR chain and the transmembrane domain. In some embodiments, a hinge region may be included between the extracellular constant domain and transmembrane domain. Hinge regions are known in the art and may be derived from immunoglobulin family members or immunoglobulin proteins. Nonlimiting examples of hinge regions include the CD8 hinge sequence, the CD28 hinge sequence, and a hybrid or combination of the two. In other embodiments, the scFv-TCR as provided herein may not include any added peptide linker or hinge sequences.

The amino acid sequence of a polypeptide such as an scFv-TCR polypeptide, may be similar but not necessarily identical to any of the amino acid sequences of the polypeptides described herein. Encompassed in the disclosure herein are polypeptides can be at least 95%, at least 96% identical, at least 97% identical, at least 98% identical, or at least 99% identical, to any of the polypeptides that make up an engineered scFv-TCR that are disclosed herein. In various embodiments, polypeptides can contain amino acid substitutions within a heavy and/or light chain variable region of the scFv moiety of an scFv-TCR polypeptide, or can include amino acid substitutions with a TCR chain constant domain sequence of an scFv-TCR polypeptide or truncated TCR subunit (e.g., the extracellular, transmembrane, or intracellular portions of the TCR constant region of the engineered TCR polypeptide. In some embodiments, the amino acid substitutions comprise one or more conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference in its entirety. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Further, one of skill in the art can consider, based on knowledge and comparison of conserved protein domains, for example, among proteins with similar function as well as, for example, known crystal structures and modeling, which regions of a protein are unlikely to result in disrupting a desired function of the protein and may therefore be modified without deleterious affects.

### Single Chain Variable Fragment Antibody (ScFv)

The scFv moiety of an engineered TCR subunit as provided herein is an scFv that specifically binds a target antigen, which can be, for example, a protein of an infectious agent, such as a virus, bacterium, or parasite, a tumor specific antigen, a tumor associated antigen, or a checkpoint protein. A tumor specific antigen can be, for example, a mutated or aberrantly spliced protein expressed by a tumor, abnormal glycosylation moieties, or a protein expressed by a tumor that is not expressed by other tissues. Tumor associated antigens include antigens overexpressed by a tumor that have restricted expression in normal tissues. Nonlimiting examples of TSAs and TAAs that may be specifically bound by an scFv include, without limitation, BCMA, CD19, CD20, CD22, CD38, CD44, CD123, CD125, CEA, claudin 18.2, EGFR VIII, ErbB2, GD2, GPC3, HER2 mesothelin, MUC1, PSMA, ROR1, TROP2, and VEGFR. In some examples an scFv of an engineered TCR or TCR polypeptide can specifically bind BCMA, CD19, CD20, CD22, CD38, CD123, Claudin 18.2, EGFRVIII, GPC3, mesothelin, MUC1, or PSMA.

Checkpoint proteins may be expressed on cancer cells, leading to downregulation of the immune response. ScFvs of the scFv-TCRs provided herein can in some embodiments be directed toward checkpoint proteins such as, for example, PD-L1, PD-L2, CTLA-4, TIM-3, LAG-3, TIGIT, BTLA, and VISTA. In some examples, an scFv of an scFv-TCR polypeptide specifically binds PD-L1 or B7H3.

In some embodiments the scFv of a scFv-TCR specifically binds PD-L1. Many PD-L1 antibodies are known (see, for example, U.S. Pat. Nos. 9,175,082 and 10,118,963) and may be used in designing an scFv-TCR as disclosed herein. In illustrative embodiments provided herein the scFv has a heavy chain variable region and light chain variable region derived from antibody SH1E2 (U.S. Pat. No. 10,058,609), and has a heavy chain variable region having at least 95% identity to SEQ ID NO:1 and a light chain variable region having at least 95% identity to SEQ ID NO:2.

The heavy chain variable region and light chain variable region are preferably joined by a linker, such as a GS linker, e.g., a (G4S) 3 linker (SEQ ID NO:3), in either the heavy chain-linker-light chain configuration (e.g., SEQ ID NO:4) or light chain-linker-heavy chain configuration (e.g., SEQ ID NO:5). Various other linkers may be used to connect the heavy and light chain regions of the ScFv, which generally include a number of glycine (G) residues for flexibility and can include other polar amino acids such as serine(S) to enhance solubility. For example, a linker used in an scFv can have multimers of (G4S) ranging from 2-20 units, or can have the alternative linkers of SEQ ID NO:44 (GGGSGGGSGGGSGGGSG) or SEQ ID NO:45 ((GGGSE)n) where n ranges from 1 to 20, or variations thereof, as nonlimiting examples.

In some embodiments, the scFv specifically binds PD-L1 and comprises a heavy chain variable region comprising a heavy chain complementarity determining region (HCDR1) comprising SEQ ID NO:46, an HCDR2 comprising SEQ ID NO:47, and an HCDR3 comprising SEQ ID NO:48, and a light chain variable region comprising a light chain complementarity determining region (LCDR1) comprising SEQ ID NO:49, an LCDR2 comprising SEQ ID NO:50, and an LCDR3 comprising SEQ ID NO:51. In some embodiments, the scFv that specifically binds PD-L1 comprises a heavy chain variable region having at least 95% identity to SEQ ID NO:1 and a light chain variable region having at least 95% identity to SEQ ID NO:2, optionally wherein the heavy chain variable region comprises the sequence of SEQ ID NO:1 and the light chain variable region comprises the sequence of SEQ ID NO:2. In some embodiments, the scFv comprises an amino acid sequence having at least 95% identity to SEQ ID NO:4, optionally wherein the scFv comprises the sequence of SEQ ID NO:4.

In some embodiments, the scFv specifically binds CD19 and comprises a heavy chain variable region having at least 95% identity to SEQ ID NO:58 and a light chain variable region having at least 95% identity to SEQ ID NO:59, optionally wherein the heavy chain variable region comprises the sequence of SEQ ID NO:58 and the light chain variable region comprises the sequence of SEQ ID NO:59. In some embodiments, the scFv comprises an amino acid sequence having at least 95% identity to SEQ ID NO:60, optionally wherein the scFv comprises the sequence of SEQ ID NO:60.

An scFv moiety of an scFv-TCR, for example, an scFv-TCRγ or scFv-TCRδ, can include an N-terminal heavy chain variable region of an antibody followed by a linker and then followed by a light chain variable region of the antibody, or can include an N-terminal light chain variable region of an antibody followed by a linker and then followed by a heavy chain variable region of the antibody. In some embodiments described herein, an anti-PD-L1 scFv of an anti-PD-L1 scFv-TCR comprises SEQ ID NO:4 or comprises an amino acid sequence that has at least 95%, at least 96%, at least 97%, at least 98%, or at least 99% identity to SEQ ID NO:4. In alternative embodiments described herein, an anti-PD-L1 scFv of an anti-PD-L1 scFv-TCR comprises SEQ ID NO:5 or comprises an amino acid sequence that has at least 95%, at least 96%, at least 97%, at least 98%, or at least 99% identity to SEQ ID NO:5.

### Nucleic Acid Molecules

Nucleic acid molecules encoding the described engineered scFv-TCR polypeptides are also provided herein.

In one aspect, provided herein are one or more recombinant nucleic acid molecules that encode an ScFv γδTCR such as any disclosed herein; i.e., one or more recombinant nucleic acid molecules that encode a first subunit of an ScFv γδTCR and a second subunit of the ScFv γδTCR, such as any disclosed herein.

In some embodiments, one or more nucleic acid molecules encode a first engineered TCR subunit as described hereinabove that comprises or consists essentially of (from N to C terminus) an ScFv fused to a TCRδ connecting peptide (SEQ ID NO:14) or a peptide having at least 95% identity thereto, followed by a TCRδ transmembrane domain (SEQ ID NO:15) or an amino acid sequence having at least 95% identity thereto. The encoded first engineered TCR subunit is preferably a precursor subunit that includes an N-terminal signal peptide. The one or more nucleic acid molecules further encode a second engineered TCR subunit that includes (from N to C terminus) the connecting peptide of a TCRγ subunit (SEQ ID NO:9) or a peptide having at least 95% identity thereto; the transmembrane domain of the TCRγ subunit (SEQ ID NO:10) or a transmembrane domain having at least 95% identity thereto; and the intracellular domain of the TCRγ subunit (SEQ ID NO:11) or an intracellular domain having at least 95% identity thereto. The encoded second engineered TCR subunit can be a precursor subunit that includes an N-terminal signal peptide.

Signal peptides are well-known in the art and can be or can be derived from a signal peptide of a secreted or membrane-directed polypeptide. Nonlimiting examples of signal peptides include SEQ ID NO:8, SEQ ID NO:13, SEQ ID NO:22, and SEQ ID NO:23.

One or more nucleic acid molecules as provided herein can include a nucleic acid sequence encoding a first engineered scFv-TCR polypeptide and a nucleic acid sequence encoding a second engineered TCR polypeptide where the first and second polypeptides, when synthesized by a cell, can associate with one another to form an scFv-TCR.

In some embodiments, two nucleic acid molecules can together encode a core TCR having first and second engineered TCR polypeptides, where, for example, the first nucleic acid molecule encodes a first scFv-TCR polypeptide and the second nucleic acid molecule encodes a truncated TCR polypeptide, where the first and second engineered TCR polypeptides, when produced by a host cell that includes the two nucleic acid molecules, associate with one another in a core TCR complex. Each of the two nucleic acid molecules can include a promoter operably linked to the sequence encoding the engineered TCR polypeptide. Examples of promoters that may be used include, without limitation, without limitation, a CMV promoter (e.g., SEQ ID NO:36), a CAG promoter, an EF1α promoter, a retroviral promoter, an HTLV promoter, an EF1α/HTLV hybrid promoter, and a JeT promoter (e.g., SEQ ID NO:35). The constructs can also optionally include a polyadenylation sequence, such as, for example, a BGH, SV40, HGH, or RBG polyadenylation sequence. In various embodiments cells may be transfected or transduced with the two nucleic acid molecules sequentially or simultaneously (e.g., in the same transfection).

In other embodiments, a single nucleic acid molecule can encode both engineered TCR polypeptides of a scFv-TCR. In some embodiments, each polypeptide-encoding sequence on the single nucleic acid molecule may be operably linked to its own promoter. In alternative embodiments, the two polypeptide-encoding sequences may be linked in a single transcriptional unit, for example by an IRES or 2A sequence, and may be operably linked to a single promoter. Nonlimiting examples of 2A sequences include SEQ ID NO:12, SEQ ID NO:52, SEQ ID NO:53, and SEQ ID NO:54.

In some embodiments, a combination of two nucleic acid molecules encode a scFv-TCR. In some embodiments, a first nucleic acid molecule encodes a scFv-TCRγ subunit and a second nucleic acid molecules encode an NT-TCRδ subunit.

In some embodiments, a nucleic acid molecule comprises (1) a promoter operably linked to the sequence encoding the scFv-γTCR and (2) a promoter operably linked to the sequence encoding the NT-TCRδ subunit. In some embodiments, the nucleic acid molecules comprise a combination of (1) a first nucleic acid molecule comprises a promoter operably linked to the sequence encoding the scFv-γTCR and (2) a second nucleic acid molecule comprising a promoter operably linked to the sequence encoding the NT-TCRδ subunit. In some embodiments, the sequence encoding the scFv-γTCR subunit and the sequence encoding the NT-TCRδ subunit are linked in the same open reading frame by a 2A sequence, and the nucleic acid molecule comprises a single promoter operably linked to the sequence encoding the scFv-γTCR subunit and the N-terminally truncated TCRδ subunit.

Specifically provided herein in some embodiments are constructs that encode an scFv-TCRγ polypeptide and do not encode an additional engineered TCR subunit polypeptide and constructs that encode an scFv-TCRδ polypeptide as disclosed herein but do not an additional engineered TCR subunit polypeptide.

A nucleic acid molecule as provided herein can be RNA, DNA, or a mixture of RNA and DNA, and can also include synthetic nucleotides with non-natural backbones such as those of PNAs, LNA, and the like. One or more nucleic acid molecules as provided herein can be in a vector, for example, a plasmid or an adenoviral, AAV, retroviral, or lentiviral vector. The vector can include one or more of integration or recombination sequences, autonomous replication sequences, and selectable markers. Alternatively, a nucleic acid molecule as provided herein can also be a linear fragment, which may be single-stranded or double-stranded. In some embodiments, a nucleic acid molecule as provided herein includes a construct encoding one or more scFv-TCR subunit polypeptides, where the DNA molecule includes homology arms flanking the TCR subunit-encoding sequences. The homology arms can be sequences of a genetic locus, such as but not limited to a TCRα (TRAC) or TCRβ (TRBC) gene.

In some embodiments, one or more nucleic acid molecules includes homology regions of the human genome flanking the open reading frame that encodes one or more scFv-TCR polypeptides. In some embodiments, the nucleic acid molecule is a plasmid. In some embodiments, the nucleic acid molecule is a linear nucleic acid molecule. In some embodiments, the nucleic acid molecule includes one or more modified nucleotides. For example, a nucleic acid molecule designed to be transfected into a population of cells for Cas-mediated integration into the genome can in some embodiments be a nucleic acid fragment which may be double-stranded or single-stranded, that may include one or more modified nucleotides.

### Host Cells

Also provided are transgenic cells that include one or more nucleic acid molecules encoding any of the scFv-TCR or an scFv-TCR subunit as described herein.

For example, a host cell provided herein can include an exogenous nucleic acid molecule that encodes an scFv-TCRγ polypeptide such as any described hereinabove, such as, for example, a polypeptide that includes an scFv moiety that specifically binds a tumor associated antigen or checkpoint inhibitor protein connected to a transmembrane domain and intracellular domain of a TCRγ polypeptide, optionally where the scFv is connected to a portion of a TCRγ polypeptide (having the amino acid sequence of a native TCRγ polypeptide or an amino acid sequence having at least 95% identity thereto) that includes the connecting peptide, transmembrane domain, and intracellular domain of a native TCRγ polypeptide or an amino acid sequence having at least 95% identity thereto.

Further provided herein are cells that include an exogenous nucleic acid molecule that encodes an scFv-TCRγ polypeptide, where the cells do not include an exogenous nucleic acid sequence encoding an engineered or nonengineered TCRδ polypeptide. For example, in some embodiments transgenic host cells are provided that include an exogenous nucleic acid sequence that encodes an scFv-TCRγ polypeptide as disclosed herein but do not include an exogenous nucleic acid sequence that encodes a polypeptide that includes a TCRδ polypeptide or a polypeptide that includes at least a portion of a TCRδ constant region, such as, for example, a TCRδ connecting peptide or a TCRδ transmembrane domain, or a substantial portion of any thereof. As demonstrated in the Examples, host cells that express a single engineered TCRγδ subunit in the absence of a further engineered or nonengineered subunit, such as, for example, host cells that express an scFv-TCRγ polypeptide, but do not express an engineered TCRδ polypeptide, can exhibit potent antitumor activity.

In some embodiments, a host cell as provided herein can include a nucleic acid molecule that encodes an scFv-TCRγ polypeptide and can lack an exogenous nucleic acid sequence that encodes an engineered or non-engineered TCRδ polypeptide.

In some embodiments, host cells that express an scFv-TCRγ polypeptide, and do not include an exogenous nucleic acid sequence encoding a second TCR polypeptide, can include a disrupted TCRα (TRAC) or TCRβ (TRBC) gene. For example, a population of host cells that includes a nucleic acid molecule encoding an scFv-TCRγ polypeptide can have the TRAC or TRBC gene knocked out prior to or simultaneous with the introduction of a nucleic acid molecule encoding the scFv-TCRγ polypeptide. In some embodiments a nucleic acid molecule encoding the scFv-TCRγ polypeptide is inserted into the TRAC or TRBC gene, inactivating the TRAC or TRBC gene. In various embodiments, a population of host cells is provided in which at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% of the cell population express an scFv-TCRγ polypeptide and do not express a TCR subunit comprising at least a portion of a TCRδ chain constant region and do not express an αβTCR. In some embodiments, a population of host cells is provided in which at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% of the cell population express an scFv-TCRδ polypeptide and do not express a TCR subunit comprising at least a portion of the constant region of a TCRγ chain and do not express an αβTCR.

Host cells as provided herein can include a nucleic acid molecule encoding an engineered scFv-TCRγ subunit, where the host cells do not include an introduced nucleic acid sequence encoding an engineered or nonengineered TCRδ subunit. An engineered or nonengineered TCRδ subunit includes at least a TCRδ connecting peptide (e.g., SEQ ID NO:14) or an amino acid sequence having at least 95% identity to a TCRδ connecting peptide or a substantial portion (at least 20 amino acids) thereof and a TCRδ transmembrane domain (e.g., SEQ ID NO:15) or an amino acid sequence having at least 95% identity to a TCRδ transmembrane domain. The host cells can be T cells, for example, primary human T cells, and in various embodiments the scFv-TCRγ subunit construct has been targeted to the TCRα or TCRβ locus, where the TCRα or TCRβ gene is disrupted.

For example, a population of host cells, such as T cells, is provided where a construct encoding an engineered scFv-TCRγ subunit as described herein has been introduced into the population, and where a nucleic acid sequence encoding an engineered or non-engineered TCRδ subunit has not been introduced into the population, in which at least 10%, at least 20%, at least 30%, at least 40%, at least 50% at least 60%, at least 65%, at least 70%, at least 75%, at least 80%, at least 85%, at least 90%, or at least 95%, of the host cell population expresses the scFv-TCRγ. In various embodiments a population of host cells, such as T cells, is provided where a construct encoding an engineered scFv-TCRγ subunit has been introduced into the population, and where a nucleic acid sequence encoding an engineered or non-engineered TCRδ subunit has not been introduced into the population, in which at least 10%, at least 20%, at least 30%, at least 40%, at least 50% at least 60%, at least 65%, at least 70%, at least 75%, at least 80%, at least 85%, at least 90%, or at least 95%, of the host cell population expresses the scFv-TCRγ and does not express an engineered TCR subunit comprising a connecting peptide and transmembrane domain of a TCRδ subunit or sequences homologous thereto. For example, a population of host cells, such as T cells can include an introduced construct encoding an engineered scFv-TCRγ subunit, where the population of host cells has not been modified (e.g., transfected or transduced) to include a nucleic acid sequence encoding an engineered or non-engineered TCRδ subunit, in which at least 10%, at least 20%, at least 30%, at least 40%, at least 50% at least 60%, at least 65%, at least 70%, at least 75%, at least 80%, at least 85%, at least 90%, or at least 95%, of the host cell population expresses the scFv-TCRγ and does not express an engineered or native TCR subunit comprising a connecting peptide (SEQ ID NO:14) or transmembrane domain of a TCRδ subunit (SEQ ID NO:15) or sequences having 95% identity to SEQ ID NO:14 or SEQ ID NO:15. In various examples, a population of host cells, such as T cells, is provided where a construct encoding an engineered scFv-TCRγ subunit has been introduced into the population, and where a nucleic acid sequence encoding an engineered or non-engineered TCRδ subunit has not been introduced into the population, in which at least 10%, at least 20%, at least 30%, at least 40%, at least 50% at least 60%, at least 65%, at least 70%, at least 75%, at least 80%, at least 85%, at least 90%, or at least 95%, of the host cell population expresses the scFv-TCRγ and does not express an engineered or native TCR subunit comprising domains of a TCRδ subunit or sequences homologous thereto and does not express a native or non-native TCRαβ receptor.

In another aspect, a transgenic cell as provided herein can an scFv-TCRδ polypeptide and an N-terminally truncated TCRγ polypeptide as described hereinabove. A transgenic cell that produces an scFv-TCR polypeptide and an N-terminally truncated TCR polypeptide can include one or more nucleic acids that encode the two polypeptides of the scFv-TCR in any configuration that allows for the production of two polypeptides by the cell. For example, a host cell can include an exogenous nucleic acid molecule as described hereinabove that encodes both polypeptides of an scFv-γδTCR. In some examples, a host cell includes an exogenous nucleic acid molecule that encodes an scFv-TCRδ polypeptide and an NT-TCRγ polypeptide.

Alternatively, a host cell can include a first exogenous nucleic acid molecule that encodes a first polypeptide of an scFv-γδTCR and a second exogenous nucleic acid molecule that encodes a second polypeptide of an scFv-γδTCR. In some examples, a host cell includes a first exogenous nucleic acid molecule that encodes an scFv-TCRγ polypeptide and a second exogenous nucleic acid molecule that encodes a NT-TCRδ polypeptide.

Nucleic acid molecules encoding a first and second polypeptide of an scFv-TCRγδ as provided herein has been introduced into the cells and at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% of the cell population express the scFv-TCRγδ.

One or more exogenous (introduced) nucleic acid molecule can be integrated into the genome of any of the host cells described herein, for example, by retroviral or lentiviral transduction or by the use of TALENs, zinc finger nucleases, transposases, or CRISPR cas systems.

The host cells provided herein that include one or more exogenous nucleic acid molecules that encode one or more TCR polypeptides can include a disrupted TCRα (TRAC) or TCRβ (TRBC) gene. For example, a population of host cells that includes one or more exogenous nucleic acid molecules encoding a first and second engineered scFv-γδTCR polypeptide can have the TRAC or TRBC gene knocked out prior to or simultaneous with the introduction of a nucleic acid molecule encoding one or more scFv-TCRγδ polypeptides. In some embodiments a nucleic acid molecule encoding one or more scFv-TCRγδ polypeptides is inserted into the TRAC or TRBC gene, inactivating the gene. In various embodiments, a population of host cells is provided in which at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% of the cell population express am scFv-TCR and do not express an αβTCR.

Transgenic host cells can be prepared by transducing host cells (such as but not limited to PBMCs or T cells) with a retroviral vector carrying a nucleic acid encoding the engineered polypeptides of an scFv-TCR. For example, the transduction can be performed essentially as described in Ma et al., 2004 The Prostate 61:12-25; and Ma et al., The Prostate 74(3):286-296, 2014 (the disclosures of which are incorporated by reference herein in their entireties). The retroviral vector can be transfected into a Phoenix-Eco cell line (ATCC) using FuGene reagent (Promega, Madison, WI) to produce Ecotropic retrovirus, then harvest transient viral supernatant (Ecotropic virus) can be used to transduce PG13 packaging cells with Gal-V envelope to produce retrovirus to infect human cells. Viral supernatant from the PG13 cells can be used to transduce activated T cells (or PBMCs) two to three days after CD3 or CD3/CD28 activation. Activated human T cells can be prepared by activating normal healthy donor peripheral blood mononuclear cells (PBMC) with 100 ng/ml mouse anti-human CD3 antibody OKT3 (Orth Biotech, Rartian, NJ) or anti-CD3, anti-CD28 TransAct (Miltenyi Biotech, German) as manufacturer's manual and 300-1000 U/ml IL2 in AIM-V growth medium (GIBCO-Thermo Fisher scientific, Waltham, MA) supplemented with 5% FBS for two days. Approximately 5×10⁶ activated human T cells can be transduced in a 10 μg/ml retronectin (Takara Bio USA) pre-coated 6-well plate with 3 ml viral supernatant and centrifuged at 1000 g for about 1 hour at approximately 32° C. After transduction, the transduced T cells can be expanded in AIM-V growth medium supplemented with 5% FBS and 300-1000 U/ml IL2.

Transgenic host cells can also be prepared using non-viral methods, including well-known designer nucleases including zinc finger nucleases, TALENS or CRISPR/Cas. A transgene can be introduced into a host cell's genome using genome editing technologies such as zinc finger nuclease. A zinc finger nuclease includes a pair of chimeric proteins each containing a non-specific endonuclease domain of a restriction endonuclease (e.g., FokI) fused to a DNA-binding domain from an engineered zinc finger motif. The DNA-binding domain can be engineered to bind a specific sequence in the host's genome and the endonuclease domain makes a double-stranded cut. The donor DNA carries the transgene, for example any of the nucleic acids encoding a scFv-TCR or scFv-TCR construct described herein, and flanking sequences that are homologous to the regions on either side of the intended insertion site in the host cell's genome. The host cell's DNA repair machinery enables precise insertion of the transgene by homologous DNA repair. Transgenic mammalian host cells have been prepared using zinc finger nucleases (U.S. Pat. Nos. 9,597,357, 9,616,090, 9,816,074 and 8,945,868). A transgenic host cell can be prepared using TALEN (Transcription Activator-Like Effector Nucleases) which are similar to zinc finger nucleases in that they include a non-specific endonuclease domain fused to a DNA-binding domain which can deliver precise transgene insertion. Like zinc finger nucleases, TALEN also introduce a double-strand cut into the host's DNA.

Transgenic host cells can be prepared using CRISPR (Clustered Regularly Interspaced Short Palindromic Repeats). CRISPR employs a Cas endonuclease coupled to a guide RNA for target specific donor DNA integration. The guide RNA includes a conserved multi-nucleotide containing protospacer adjacent motif (PAM) sequence upstream of the gRNA-binding region in the target DNA and hybridizes to the host cell target site where the Cas endonuclease cleaves the double-stranded target DNA. The guide RNA can be designed to hybridize to a specific target site. Similar to zinc finger nuclease and TALEN, the CRISPR/Cas system can be used to introduce site specific insertion of donor DNA having flanking sequences that have homology to the insertion site. Examples of CRISPR/Cas systems used to modify genomes are described for example in U.S. Pat. Nos. 8,697,359, 10,000,772, 9,790,490, and U.S. Patent Application Publication No. US 2018/0346927.

As exemplified herein, CRISPR/Cas methods that simultaneous knock out an endogenous gene of the host cells when an exogenous construct is inserted at the locus can be employed (see, for example, US 2020/0224160 and WO 2020/185867, both of which are incorporated by reference herein in their entireties). Transgenic host cells produced by such methods can incorporate a nucleic acid molecule encoding scFv-TCR polypeptides while losing expression of, for example, the TRAC (TCR alpha chain) gene. The transgenic host cells can be T cells, for example, can be CD3+ cells (expressing the T cell receptor) isolated from PBMCs prior to transfection. Further, following transfection with an scFv-TCR construct and Cas RNP targeting the TRAC locus, the transfected culture can be expanded and then CD3+ cells (i.e., cells that express the T cell receptor) can be depleted, for example, using magnetic beads conjugated to a CD3 antibody, resulting in cultures enriched for cells in which the TRAC gene has been knocked out by incorporation of the construct encoding the engineered TCR polypeptide(s).

The donor DNA can include for example any of the nucleic acids encoding a scFv-TCR polypeptides described herein, including an scFv-TCRγ polypeptide. Various delivery methods to co-deliver into the host cell the donor DNA with the zinc finger nuclease, TALEN or CRISPR/Cas system may be used, including, without limitation, electroporation, LNPs, nucleofection, and lipofection. Other methods of integrating a construct encoding a scFv-TCR polypeptide or scFv-TCR construct can include using transposases such as Sleeping Beauty or Piggy Back, or transposases or genome-modifying enzymes derived therefrom.

Provided herein are cell cultures wherein at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% of the cells of the culture express an scFv-TCR polypeptide, such as any disclosed herein, including, for example, an scFv-TCRγ polypeptide. The cell cultures can be T cell cultures, for example, primary T cell cultures and can be cultures of primary human T cells. In various embodiments less than 10%, less than 8%, less than 7%, less than 5%, less than 3%, less than 2%, less than 1.5%, less than 1%, or less than 0.5% of the T cell cultures are CD3+ cells, e.g., less than 10%, less than 8%, less than 7%, less than 5%, less than 3%, less than 2%, less than 1.5%, less than 1%, or less than 0.5% of the cells of the culture express the endogenous T cell receptor. Provided herein is a population of T cells in which at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% of the cells of the culture express an scFv-TCR as disclosed herein and do not express the endogenous T cell receptor and a pharmaceutical composition comprising such a cell population. The cell population can be provided as a composition formulated for intravenous infusion or injection, for example.

In various embodiments, a host cell can be introduced with an expression vector or nucleic acid fragment in which a promoter is operably linked to a nucleic acid sequence encoding a scFv-TCR or engineered TCR polypeptide thereby generating a transfected/transformed host cell which is cultured under conditions suitable for expression of the scFv-TCR or engineered TCR polypeptide by the transfected/transformed host cell.

Typically, a host cell is a cultured cell that can be transformed or transfected with a polypeptide-encoding nucleic acid, which can then be expressed in the host cell. The phrase “transgenic host cell” or “recombinant host cell” can be used to denote a host cell that has been introduced (e.g., transduced, transformed, or transfected) with a nucleic acid to be expressed. A host cell also can be a cell that comprises the nucleic acid but does not express it at a desired level unless a regulatory sequence is introduced into the host cell such that it becomes operably linked with the nucleic acid. It is understood that the term host cell refers not only to the particular subject cell but also to the progeny or potential progeny of such a cell. Because certain modifications may occur in succeeding generations due to, e.g., mutation or environmental influence, such progeny may not, in fact, be identical to the parent cell, but are still included within the scope of the term as used herein. A host cell, or a population of host cells, harboring a vector (e.g., an expression vector) operably linked to at least one nucleic acid encoding one or more engineered TCR polypeptides that make up a engineered TCR polypeptide TCR are described herein.

In various embodiments the host cell or the population of host cells can comprise T lymphocytes (e.g., T cells, regulatory T cells, gamma-delta T cells, and cytotoxic T cells), NK (natural killer) cells, macrophages, dendritic cells, mast cells, eosinophils, B lymphocytes, monocytes. In some embodiments, the NK cells comprise cord blood-derived NK cells, or placental derived NK cells. A population of host cells can comprise human T lymphocytes or human NK cells, for example, can be primary human T cells or NK cells. In various exemplary embodiments provided herein, primary human scFv-TCR-T cells, i.e., primary human T cells transfected with scFv-TCR polypeptide construct(s) and expressing a scFv-TCR polypeptide, are provided. The host cells provided herein can be a cell population where the population of host cells has been positively selected (for example, selected by magnetic beads conjugated to a binding partner or by the use of other capture reagents of formats and/or by flow cytometry) and/or may be a population of host cells from which some cell types have been removed or depleted (subtracted) for example, by magnetic bead capture, flow cytometry, or other methods. The cells may be selected or enriched by use of a binding partner that is bound by the expression of the construct (scFv-TCR or scFv-TCR polypeptide) transfected into cells. Further, a population of host cells may be selectively expanded, for example, by culturing in the presence of a binding partner for the scFv-TCR polypeptide expressed by the transfected cells of the population, or in the presence of cells that express the binding partner.

Cell populations that comprise transgenic cells expressing a scFv-TCR or scFv-TCR polypeptide (e.g., an scFv-TCRγ polypeptide) as provided herein can be provided as pharmaceutical compositions, for example, for injection or infusion. In some embodiments, the cells are T cells or NK cells. In some embodiments, the cells are T cells that do not express an endogenous T cell receptor. In some embodiments, a pharmaceutical preparation includes a population of primary T cells, such as human primary T cells, where at least 20% of the cells of the population express an scFv-TCR polypeptide and less than 5%, less than 2%, less than 1%, or less than 0.5% of the cells of the population express an endogenous T cell receptor.

A population of scFv-TCR or scFv-TCR polypeptide-expressing cells, such as any described herein, for example scFv-TCRγ-T cells, can be provided for infusion (e.g., intravenous or intraarterial infusion) or injection (e.g., one or more intravenous, intratumoral, or subcutaneous injections). The cell formulation can be frozen for storage and shipping and can optionally provide cells for multiple treatments with the same cell preparation. The cells can be packaged as a product or kit in vials, bags, or tubes, for example. Instructions (e.g., written instructions) may be provided on the use of the cells.

In various embodiments cell populations are provided in which the cells have been transfected or transduced with a nucleic acid construct that encodes a scFv-TCRγ polypeptide, where at least 20%, at least 30%, at least 40%, at least 50%, at least 60%, at least 70%, at least 80%, or at least 90% of the cells express the scFv-TCRγ polypeptide as assessed by flow cytometry. In some embodiments, at least a portion of the cells of the population do not express the T cell receptor, for example, are knocked out in the TRAC gene. The population of T cells having at least 20% of the cells expressing scFv-TCR and may be depleted of CD3 (T cell receptor) positive cells, e.g., less than 5%, less than 2%, less than 1%, or less than 0.5% of the cell population can express the T cell receptor.

In some embodiments the cells are T cells. In some embodiments the cells are primary T cells (scFv-TCR cells).

### Methods of Treatment

Provided herein are methods of treating cancer comprising administering to a subject a therapeutically-effective amount of a population of cells as provided herein that express a scFv-TCR or scFv-TCRγ polypeptide, such as any described herein. The cells may be T cells and at least 10% of the cell population can express the scFv-TCR or scFv-TCRγ polypeptide.

The cancer or tumor includes cancer of the prostate, breast, ovary, head and neck, bladder, skin, anus, rectum, pancreas, lung (including non-small cell lung and small cell lung cancers), bone, leiomyoma, brain (including glioma and glioblastoma), esophagus, liver, kidney, stomach, colon, cervix, uterus, endometrium, vulva, larynx, vagina, bone, nasal cavity, paranasal sinus, nasopharynx, oral cavity, oropharynx, hypolarynx, salivary glands, ureter, urethra, penis and testis.

In further embodiments, the cancer comprises a hematological cancer, including leukemias, lymphomas, myelomas, and B cell lymphomas. Hematologic cancers include multiple myeloma (MM), non-Hodgkin's lymphoma (NHL) including Burkitt's lymphoma (BL), B chronic lymphocytic leukemia (B-CLL), systemic lupus erythematosus (SLE), B and T acute lymphocytic leukemia (ALL), acute myeloid leukemia (AML), chronic lymphocytic leukemia (CLL), diffuse large B cell lymphoma, chronic myelogenous leukemia (CML), hairy cell leukemia (HCL), follicular lymphoma, Waldenstrom's Macroglobulinemia, mantle cell lymphoma, Hodgkin's Lymphoma (HL), plasma cell myeloma, precursor B cell lymphoblastic leukemia/lymphoma, plasmacytoma, giant cell myeloma, plasma cell myeloma, heavy-chain myeloma, light chain or Bence-Jones myeloma.

In some embodiments the cancer is neuroblastoma, glioblastoma, glioma, a neurocytoma, astrocytoma, medulloblastoma, melanoma, breast cancer, small-cell lung cancer, pancreatic cancer, medullablastoma, osteosarcoma or other soft tissue sarcoma, a hematological cancer, bladder cancer, a chondrosarcoma, colorectal cancer, esophageal cancer, gastric cancer, head and neck cancer, kidney cancer, a leiomyoma, a leiomyosarcoma, liver cancer, lung cancer, mesothelioma, an osteosarcoma, ovarian cancer, prostate cancer, rhabdosarcoma, renal cancer, testicular cancer, or uterine cancer.

The cells can be administered for at least about 30 seconds, 1 minute, 10 minutes, 30 minutes, 1 hour, 2 hours, 3 hours, 4 hours, 5 hours, 10 hours, 12 hours, or 24 hours in a single dosing. Cells can be administered in a single dose or in multiple doses over minutes, hours, days, weeks, or months. A population of cells as described herein can be administered before, during, or after the occurrence of a disease or condition, and the timing of administering a pharmaceutical composition containing the cell population can vary. The initial administration can be via any route practical, such as by any route described herein using any formulation described herein. In some examples, the administration is an intravenous administration. In some embodiments, one or multiple dosages of the T-cell population can be administered after the onset of a cancer and optionally for a length of time necessary for the treatment of the disease.

### EXAMPLES

The following examples can be used to further understand embodiments of the present disclosure. The examples are meant to be illustrative and should not be construed as limiting the scope of the present teachings in any way.

### Example 1: Isolation of Human PBMC Cells and Primary T Cells

Primary human T cells were isolated from healthy human donors either from buffy coats (San Diego blood bank), fresh blood, or leukapheresis products (STEMCELL Technologies, Vancouver, CA or HemaCare, Northridge, CA). Peripheral blood mononuclear cells (PBMCs) were isolated by density gradient centrifugation and used directly or frozen in CyroStor CD10 (Sigma-Aldrich) prior to isolation of T cells.

T cells were isolated from PBMCs by magnetic negative selection using EASYSEP Human T Cell Isolation Kit (STEMCELL Technologies). After isolation or thawing of frozen cells, T cells were activated with DYNABEADS Human T-Expander CD3/CD28 (Thermo Fisher Scientific, Waltham, MA, USA) according to manufacturer's instructions for three days, after which the beads were magnetically removed and cells were cultured one more day prior to transfection.

### Example 2: Primary T Cell Culture

Primary T cells were cultured in GIBCO OpTmizer™ CTS™ T-Cell Expansion serum-free medium (ThermoFisher) supplemented with 5% human AB serum (Valley Biomedical, Winchester, VA, USA) and 300 U/mL IL-2 (Miltenyi Biotec) at a density of approximately 10⁶ cells per mL. Following transfection, T cells were cultured in media with IL-2 at 300 U/mL.

### Example 3: scFv-TCR Constructs

An αPDL1-δ-TCRγδ construct (SEQ ID NO:6) was generated that included a sequence encoding, as a first engineered TCR subunit, a chimeric anti-PD-L1 scFv-TCRδ subunit (αPDL1-TCRδ), and as a second engineered TCR subunit, an N-terminally truncated TCRγ subunit (NT-TCRγ). The construct was configured to have the sequence encoding the NT-TCRγ subunit followed by the sequence encoding the αPDL1-TCR& subunit, where the two subunits were connected by a sequence encoding a T2A peptide to allow the two subunits to be transcribed from the same promoter. The NT-TCRγ included a TCRγ9 extracellular connecting peptide (SEQ ID NO:9) at the N-terminus followed by the TCRγ9 subunit transmembrane domain (SEQ ID NO:10) and the TCRγ9 subunit intracellular domain (SEQ ID NO:11). The sequence encoding the mature NT-TCRγ subunit (SEQ ID NO:17) was preceded by a sequence encoding a signal peptide (SEQ ID NO:8) positioned at the N-terminus of the protein. The sequence encoding the second subunit was followed by a sequence encoding a T2A peptide (SEQ ID NO:12) which was in turn followed by a sequence encoding the first engineered TCR subunit, which included an anti-PD-L1 scFv antibody (SEQ ID NO:4) having the heavy chain variable region of SEQ ID NO:1 linked via a (G4S)₃ linker (SEQ ID NO:3) to the light chain variable region of the antibody (SEQ ID NO:2). The sequence encoding the anti-PD-L1 scFv antibody was followed by a sequence encoding a TCRδ2 extracellular connecting peptide (SEQ ID NO:14), which was followed by the TCRδ2 transmembrane domain (SEQ ID NO:15). The sequence encoding the mature αPDL1-TCRδ subunit (SEQ ID NO:19) was preceded by a sequence encoding a signal peptide (SEQ ID NO:13) positioned at the N-terminus to generate a sequence encoding a precursor αPDL1-TCRδ subunit (signal peptide-anti-PD-L1 scFv-TCRδ connecting peptide-TCRδ transmembrane domain; SEQ ID NO:18).

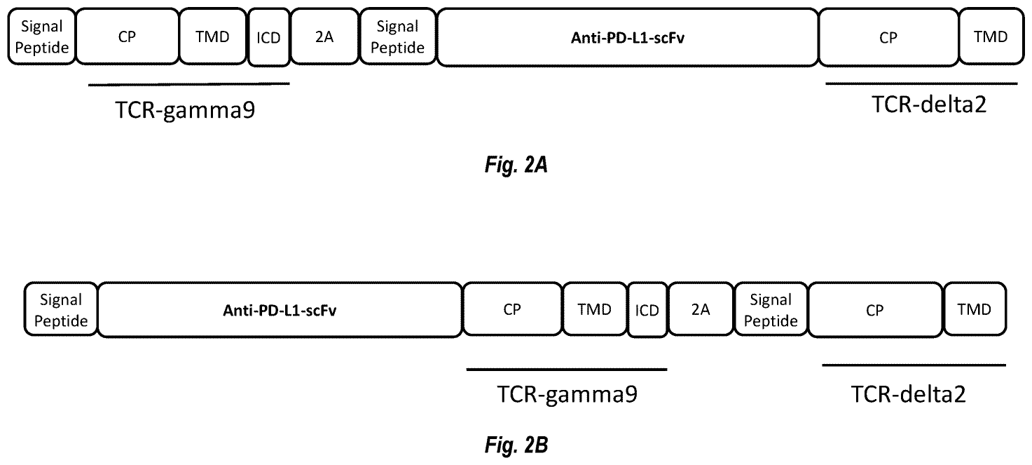

The entire αPDL1-δ-TCRγδ construct (FIG. 2A) encoding both engineered TCR subunits connected by the T2A peptide (SEQ ID NO:12), was given the name GD102 and cloned in a vector downstream of the JeT promoter (SEQ ID NO:35; U.S. Pat. No. 6,555,674).

A second anti-PD-L1 scFv-TCRγδ construct (SEQ ID NO:20; FIG. 2B) included a sequence encoding a first subunit (αPDL1-TCRγ) in which a sequence encoding the same anti-PD-L1 scFv antibody (SEQ ID NO:4) was followed by the extracellular connecting peptide of the TCRγ9 subunit (SEQ ID NO:9), which was followed by the TCRγ9 subunit transmembrane domain (SEQ ID NO:10) and then the TCRγ9 subunit intracellular domain (SEQ ID NO:11). The sequence encoding the mature αPDL1-TCRγ subunit (SEQ ID NO: 25) was preceded by a sequence encoding a signal peptide (SEQ ID NO:22) positioned at the N-terminus of the ScFv moiety of the polypeptide. The nucleic acid sequence encoding the first subunit precursor was followed by a sequence encoding a T2A “self-cleaving” peptide (SEQ ID NO:12), and the first subunit precursor-encoding sequence was then followed by a sequence encoding the second subunit of the αPDL1-γ-TCRγδ. The second subunit was essentially an N-terminally truncated TCRδ2 chain, and included the TCRδ2 extracellular connecting peptide (SEQ ID NO:14), followed by the TCRδ2 transmembrane domain (SEQ ID NO:15). The sequence encoding the mature second subunit (SEQ ID NO:27) was preceded also by a sequence encoding a signal peptide (SEQ ID NO:23) positioned at the N-terminus of the encoded NT-TCRδ2 precursor protein (SEQ ID NO:26). The entire αPDL1-γ-TCRγδ construct, including sequences encoding the first and second PD-L1 scFv-TCRγδ subunits connected by the T2A peptide was given the name GD109 and cloned in a vector downstream of the JeT promoter (SEQ ID NO:35; U.S. Pat. No. 6,555,674).

A PD-L1 CAR construct was also made. The PD-L1 CAR-ζFL (SEQ ID NO:29, encoded by SEQ ID NO:28) also included the anti PD-L1 scFv antibody (SEQ ID NO:4) that included the heavy chain variable region of the SH1E2 antibody (SEQ ID NO:1) linked via a GS linker (SEQ ID NO:3) to the light chain variable region of the SH1E2 antibody (SEQ ID NO:2). Following the light chain variable region was a hinge region that included the hinge sequence of CD8 (SEQ ID NO:30) followed by the hinge sequence of CD28 (SEQ ID NO:31) which was then followed by the CD28 transmembrane domain (SEQ ID NO:32) and then by an intracellular domain that included the co-stimulatory domain of 4-1BB (SEQ ID NO:34) and the full-length signaling domain of CD3ζ (SEQ ID NO:33). The nucleic acid construct encoding the PD-L1 CAR-ζFL (SEQ ID NO:28) also included a sequence encoding a signal peptide (SEQ ID NO:22) positioned at the N-terminus of the protein. The entire PD-L1 CAR-ζFL construct (SEQ ID NO:28) was cloned in a vector downstream of a JeT promoter (SEQ ID NO:35).

TABLE 1
ScFv Constructs.

| Construct | Encoded engineered | | |
|---|---|---|---|
| Name | receptor | First Polypeptide | Second Polypeptide |
| GD102 | αPDL1-δ-TCRγδ | αPDL1-TCRδ | NT-TCRγ |
| (SEQ ID NO: 6) | (scFv-δ-TCRγδ) | (SEQ ID NO: 19) | (SEQ ID NO: 17) |
| | | (scFv-TCRδ) | |
| GD109 | αPDL1-γ-TCRγδ | αPDL1-TCRγ | NT-TCRδ |
| (SEQ ID NO: 20) | (scFv-γ-TCRγδ) | (SEQ ID NO: 25) | (SEQ ID NO: 27) |
| | | (scFv-TCRγ) | |

### Example 4. Preparation of CAR-T Cells and scFv-TCR-T Cells

T cells isolated were individually transfected with a Cas9 RNP including a guide targeting Exon 1 of the TRAC gene and a donor DNA that included engineered anti-PD-L1 scFvTCR subunits.

Activated T cells (approximately 8×10⁶ cells) were transfected with a Cas9 RNP targeting the TRAC locus and a DNA fragment that included the GD102 construct (SEQ ID NO:6) having a sequence encoding a chimeric αPDL1-TCRδ subunit (SEQ ID NO:19) and an N-terminally truncated TCRγ subunit (SEQ ID NO:17).

Another aliquot of activated T cells was transfected with a Cas9 RNP targeting the TRAC locus and a DNA fragment that included the GD109 construct (SEQ ID NO:20) having a sequence encoding a chimeric αPDL1-TCRγ subunit (SEQ ID NO:25) and an N-terminally truncated TCRδ subunit (SEQ ID NO:27).

As a control, a third aliquot of T cells was transfected with the TRAC gene-targeting RNP only (designated TRAC knockout or simply KO cells).

In a second set of transfections, separate aliquots of activated T cells (approximately 8×10⁶ cells) were transfected with nucleic acids that included the GD109 construct, the GD102 construct, and the PD-L1 CAR-ζFL construct (SEQ ID NO:28), in each case with an RNP targeting the TRAC locus. As a control, a fourth aliquot of T cells was transfected with the RNP only (TRAC knockout or KO cells).

For each construct (PD-L1 CAR-ζFL (SEQ ID NO:28), GD102 (SEQ ID NO:6), and GD109 (SEQ ID NO:20), the expression cassette having an operably linked promoter at the 5′ end was cloned in a vector between 5′ and 3′ homology arms (SEQ ID NO:37 and SEQ ID NO:38, respectively) that flanked a Cas9 target site (SEQ ID NO:39) in the TRAC gene. To generate a double-stranded donor fragment, PCR was performed using a modified forward primer (SEQ ID NO:40) and a reverse primer (SEQ ID NO:41) as described in US Patent Application Publication No. US 2020/0224160, incorporated herein by reference in its entirety, to generate a donor fragment that included the expression construct flanked by homology arms of 171 and 161 bps (SEQ ID NO:42 and SEQ ID NO:43). The resulting double stranded donor DNA fragments were 2.405, 2.141, and 2.138 Kilobases in size and were used to independently transfect activated T cells as double-stranded DNA molecules together with a Cas9 RNP targeting the T cell receptor alpha (TRAC) locus.

To transfect isolated T cells with the engineered receptor constructs, 4×10⁶ cells were mixed with a Cas9 RNP that included a tracr RNA and a guide RNA targeting exon 1 of the TRAC locus (target sequence of SEQ ID NO:39) (IDT, Coralville, IA, USA) and 5 μg of the double-stranded donor DNA (including either the PD-L1 CAR-ζFL construct, the GD102 construct, or the GD109 construct), were then added. The cells were electroporated with 1700 V. 20 ms pulse width, 1 pulse using Neon Transfection System (Thermo Fisher Scientific, Waltham, MA, USA) and 100 μL tips. As controls, one aliquot of T cells from each PBMC donor source was transfected with the Cas9 RNP but without a donor DNA fragment. In the absence of a donor DNA, the RNP will disrupt the targeted gene but no expression construct is inserted. The cells transfected with a targeting RNP but without a donor DNA are therefore referred to as TCR knockout (KO) controls. Additionally, as further controls, aliquots of T cells from each donor source were cultured with IL-2 identically to the transfected T cells but were not transfected (activated T cells or ATCs).

Following electroporation with the Cas9 RNP and donor fragment, cells were diluted into culture medium and incubated at 37° C., 5% CO₂ in OpTmizer™ T Cell Expansion SFM supplemented with 5% human AB serum (Valley Biomedical, Winchester, VA. USA) and 300U/ml IL2 in 37° C. Once cells were in expansion cultures, cell counts were obtained every 2 to 3 days and the cell concentration was maintained at 5×10⁵ (5e5) to 1×10⁶ (1e6) per mL.

### Example 5: Analysis of Receptor Expression of CAR-T Cells and scFv-TCRγδ Cells

Flow cytometry was used to analyze the transfected T cells fourteen days after transfection by cell surface staining using antibodies to CD3, TCRαβ, the variable domains of TCRδ2 and TCRγ9, CD4 and CD8, and the PD-L1 protein.

To detect the CD3ε protein and the αδTCR, aliquots of about 1×10⁵ cells were washed with DPBS/5% human serum albumin, then stained with APC/Cyanine7 anti-human CD3ε antibody (BioLegend clone UCHT1), and PerCP/Cyanine5.5 anti-human TCR a/B antibody (BioLegend clone IP26), together with antibodies to detect TCRδ2 (Brilliant Violet 421™ anti-human TCR Vδ2 antibody), TCRγ9 (PE anti-human TCR Vγ9 antibody), CD4 (FITC BioLegend anti CD4 clone RPA-T4),) and CD8 (BV605 BioLegend antiCD8a clone RPA-T8). The incubation also included Fc-fused PD-L1 protein to detect anti-PD-L1 antibody (Sino Biological). The cells were washed and flow cytometry was performed by an Attune flow cytometer (Thermo Fisher Scientific, Waltham, MA, USA).

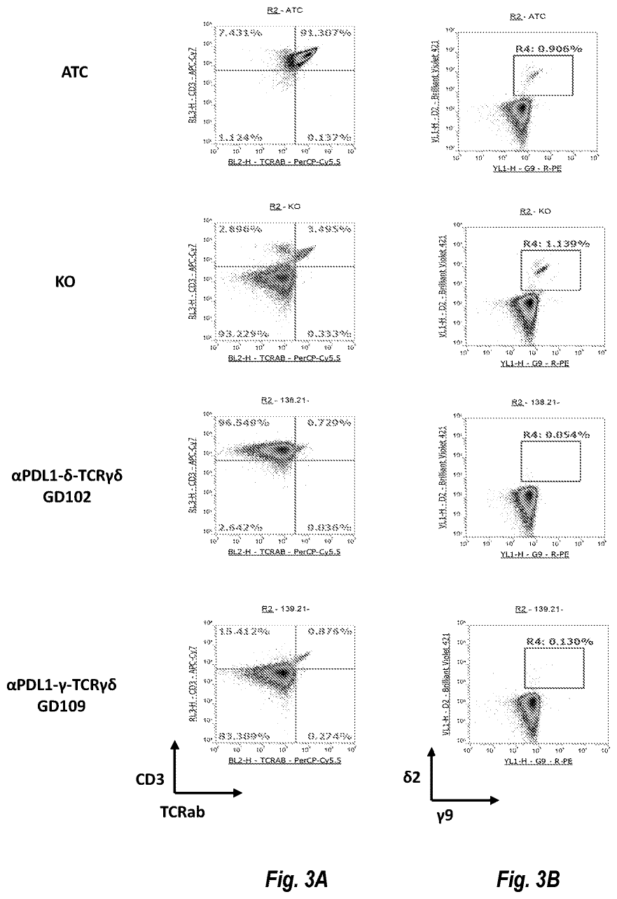

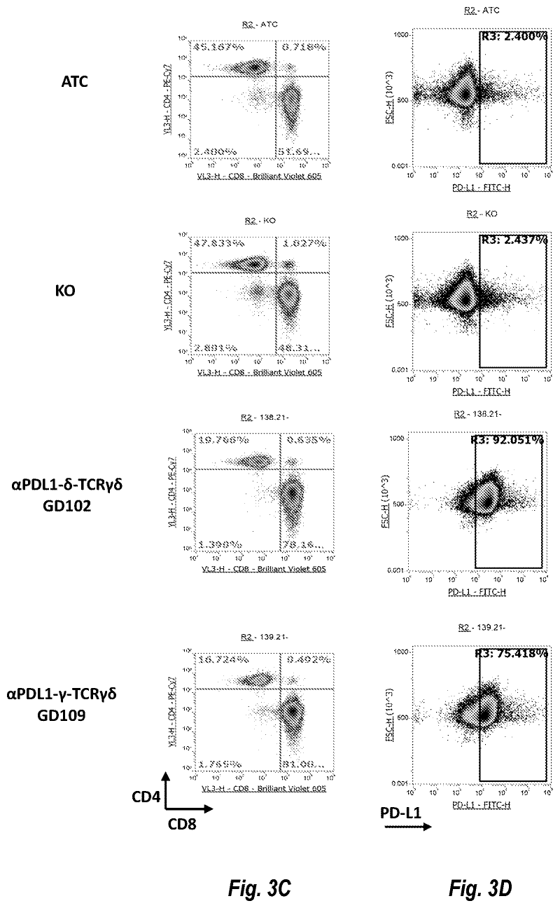

FIGS. 3A-3D show the results of flow cytometry on cells 14 days after transfection. Approximately 91% of the non-transfected ATCs expressed both the αβTCR and CD3, whereas only approximately 3.5% of the TRAC knockout (KO) cell population expressed the αβTCR, with CD38 detected on only about 6.5% of the population (FIG. 3A). In contrast, approximately 96.55% of the GD102 (αPDL1-δ-TCRγδ) transfected cell population were positive for CD38 while not expressing the αβTCR, indicating that CD3 was able to associate with the engineered TCRγδ. The GD109 (αPDL1-γ-TCRγδ) transfected cells, which also lacked αβTCR expression, were found to be largely negative for CD3ε.

FIG. 3B shows that only about 1% or less of the nontransfected (ATC), KO, GD102, or GD109 transfected cell populations expressed a native γδTCR, i.e., a TCR subunit having extracellular sequences of a native y or & TCR subunit. FIG. 3C shows that approximately 45-48% of the nontransfected ATCs or KO cells were CD4+, with about 49-53% being CD8+ cells. In contrast, approximately 78-82% of the cells transfected with the GD102 TCR construct or GD109 TCR construct were CD8+, and only about 17-20% were CD4+.

FIG. 3D shows that the majority of the cells of cultures transfected with the GD102 TCR construct expressed the GD102 construct and the majority of the cells of cultures transfected with the GD109 TCR construct expressed the GD109 construct (about 92% and 75%, respectively), as diagnosed by staining with FITC-conjugated PD-L1, whereas only a very small percentage (about 2.4%) of the nontransfected and KO populations bound PD-L1.

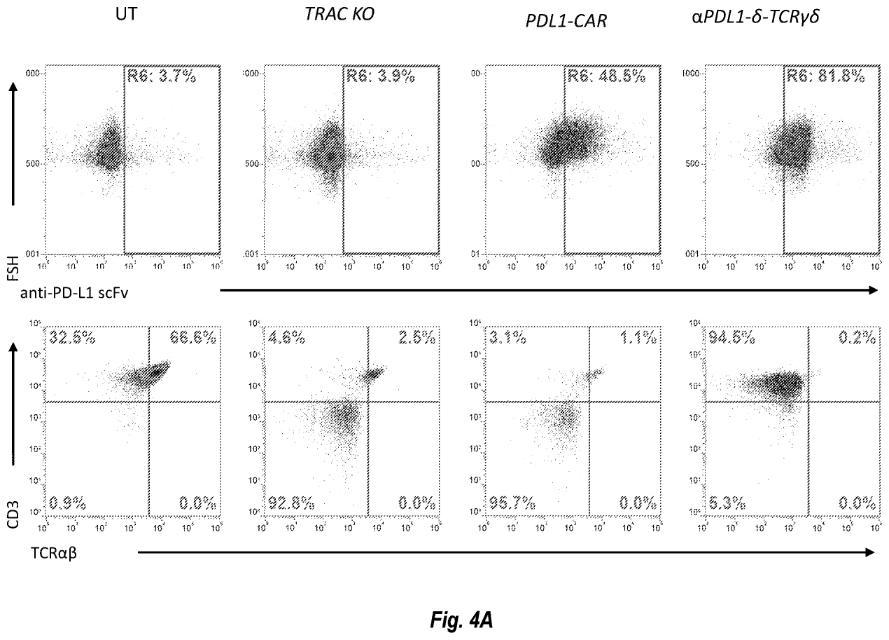

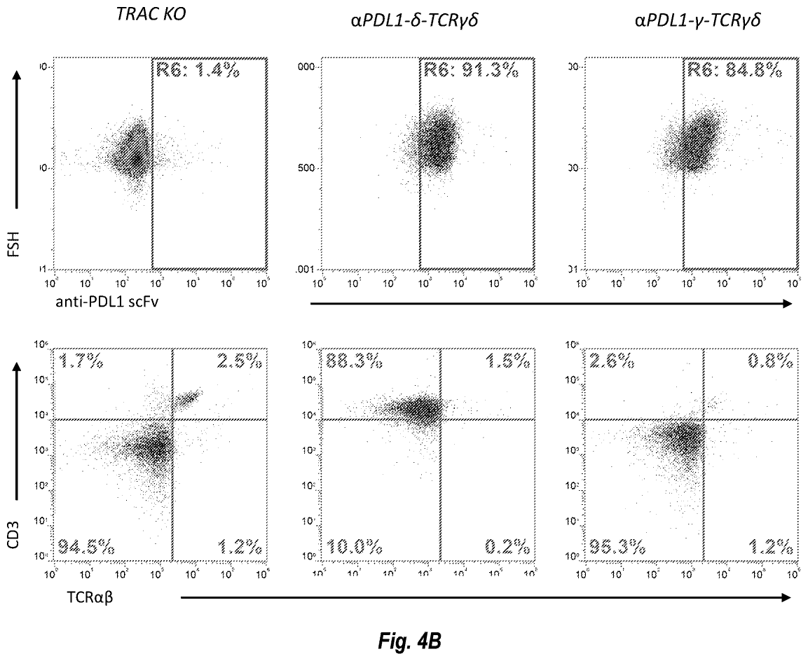

FIG. 4A compares flow cytometry results of T cells ten days after another transfection with the PDL1 CAR construct and the GD102 construct (encoding αPDL1-8-TCRγδ). Control cells that were either untransfected (UT) or TRAC knockout cells (no construct) were also analyzed. While nearly 50% of the PDL1 CAR-T cells bound PD-L1 on Day 10, the percentage of GD102 cells that bound PD-L1 was about 80%, while very few (approximately 4%) of untransfected and TRAC knockout cells bound PD-L1. None of the transfected populations expressed a significant amount of TCRαβ (which was detected on approximately 66% of nontransfected cells), and CD38 was detected on nearly all nontransfected cells but only approximately 7% of the cells transfected with the RNP only (TRAC knockout population). Fewer than 5% of cells transfected with the PDL1 CAR construct stained positively for CD38 while approximately 95% of the cells transfected with the PD-L1 GD102 stained positively for CD38, suggesting CD3 complex association with the recombinant receptor having the scFv linked to TCRδ sequences.

FIG. 4B compares flow cytometry results of T cells ten days after another transfection with the GD102 construct having the anti-PD-L1 scFv on the delta chain and the GD109 construct having the anti-PD-L1 scFv on the gamma chain. TRAC knockout control cells were also analyzed. In this analysis, approximately 91% of the GD102 cells and approximately 85% of the GD109 cells bound PD-L1 (indicating expression of the recombinant receptor) whereas the TRAC knockout control cells did not bind PD-L1. Surprisingly, although approximately 88% of the GD102 T cell population expressed CD38 (while not expressing the TCRαβ), approximately 95% of the GD109 T cell population did not express CD3ε (while not expressing TCRαβ), suggesting that CD3ε, and other CD3 subunits, assembled with the recombinant γδTCR that included the anti-PD-L1 scFv on the 8 chain polypeptide to form the TCR-CD3 complex, but did not stably assemble with the recombinant γδTCR that included the anti-PD-L1 scFv on the γ chain polypeptide.

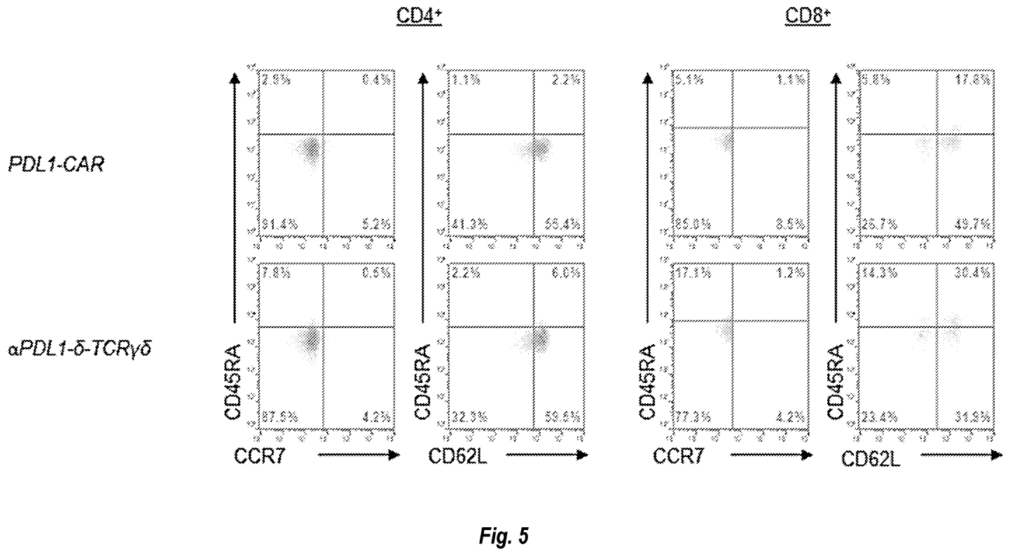

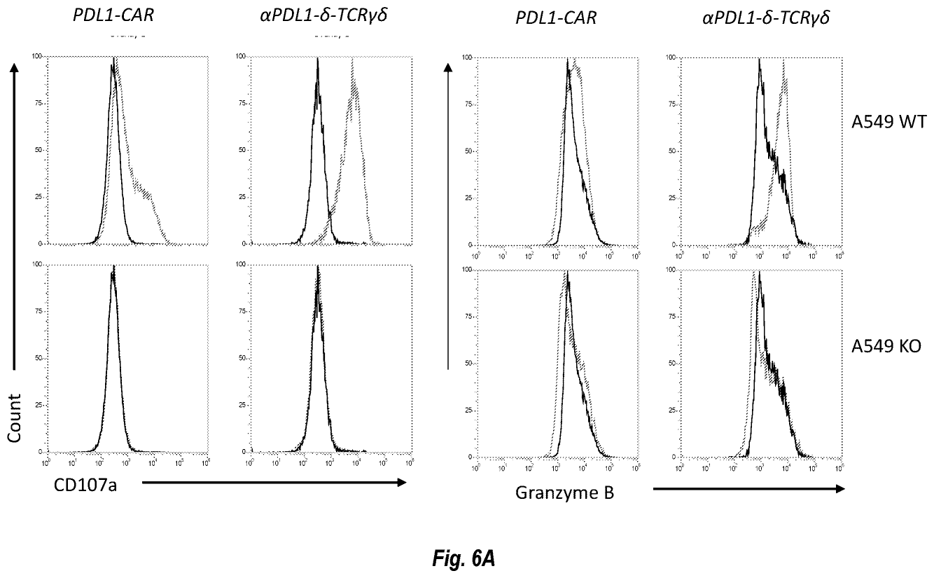

Expression of memory markers by the T cells having engineered γδTCRs were also assessed by flow cytometry. T cells were gated for CD4 and CD8 expression and examined for expression of CD45RA, CCR7, and CD62L. All antibodies were from BioLegend (CD4, clone RPA-T4; CD8, clone RPA-T8; CD45RA, clone HI100; CD62L, clone DREG-56; CCR7, clone G0443H7) FIG. 5 shows that GD102 αPDL1-δ-TCRγδ-T cells showed a similar pattern of memory T cell marker expression to PD-L1 CAR-T cells.

### Example 10. Degranulation, Cytokine Release, and Cytotoxicity Assays Using αPDL1-8-TCRγδ-T Cells

To test tumor killing function of the T cells expressing the αPDL1-δ-TCRγδ recombinant receptor, the engineered T cells were stimulated with A549 lung carcinoma cells and SK-MEL-5 melanoma cells, both of which express PD-L1.

In degranulation assays using a CD107a antibody. T cells knocked out for the αβ TCR but not expressing an engineered receptor (designated TRAC-KO), T cells expressing the PD-L1 CAR, and T cells expressing the GD102 construct encoding the αPDL1-δ-TCRγδ receptor were incubated with A549 or SK-MEL-5 target cells at a 1:1 ratio. Engineered T cells were cultured in the absence of target cells as controls. The cells were incubated at 37° C. in 5% CO₂ in the presence of the CD107a antibody for 2 hours, after which time Brefeldin A was added and the cells were cultured a further 3 hours. The cells were then harvested and surface stained. The cells were also stained with a granzyme B antibody (Becton Dickinson) after fixation and permeabilization using Cytofix/Cytoperm Fixation/Permeabilization solution (Becton Dickinson).

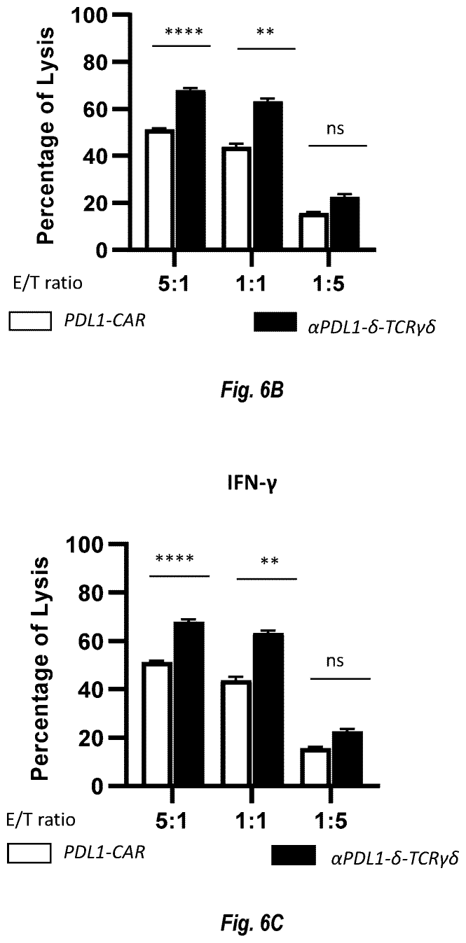

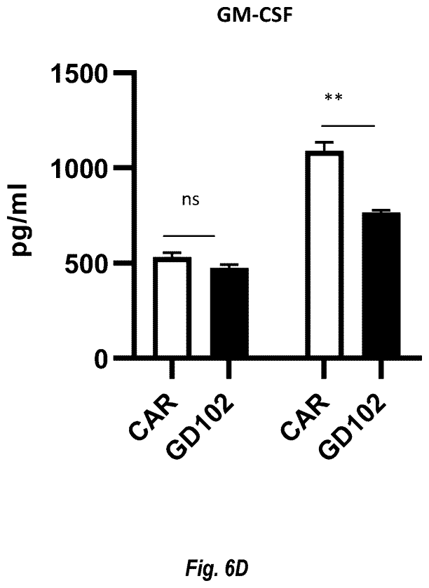

In assays with A549 wild type cells as targets, αPDL1-δ-TCRγδ-T cells showed significantly higher levels of degranulation, reflected by increased expression of CD107a and granzyme B, as compared to CAR-T cells (FIG. 6A, upper panels). On the other hand, when co-culturing with PD-L1-knockout A549 cells (FIG. 6A, lower panels), no elevated expression of granzyme B or CD107 was observed in either αPDL1-δ-TCRγδ-T or CAR-T cells, indicating that the activation of both types of engineered T cells was triggered by the targeted antigen.

Cytotoxicity assays were also performed with A549 target cell lines expressing GFP-Firefly luciferase (GFP-Fluc). Briefly, 105 tumor cells were plated per well in a 96 well round-bottom plate and engineered T cells (effector cells) were added at E:T ratios of 5:1. 1:1, 1:5, and 1:25. Cells were cocultured for 4 or 24 hours, after which time the cells were stained with fixable viability dye (ThermoFisher), washed with Binding buffer, and then stained with Annexin V and analyzed by flow cytometry. Cytotoxicity was calculated as the percent of Annexin V-positive live/dead cells gated on the target cells. The engineered αPDL1-δ-TCRγδ-T cells demonstrated efficient killing of A549 tumor cells in a dose-dependent manner, significantly outperforming CAR-T cells, with the strongest tumor killing activity recorded at the highest effector/target (E:T) ratio of 5:1 (FIG. 6B).

To measure secreted cytokines, 1×10⁵ anti-PD-L1 scFv expressing T cells were cocultured with 1×10⁵ A549 (WT) tumor cells in 96-well plate at 37° C. overnight. Supernatants were collected and cytokines were measured using IFN-γ and GM-CSF ELISA kits (Thermo Fisher) and read by Cytation5 imaging reader. The OD values were calculated, and the data were plotted using GEN5 software. FIGS. 6C and 6D show that when co-cultured with A549 WT cells, the amount of IFN-γ and GM-CSF produced by the αPDL1-δ-TCRγ8-T cells was lower than that produced by CAR-T cells.

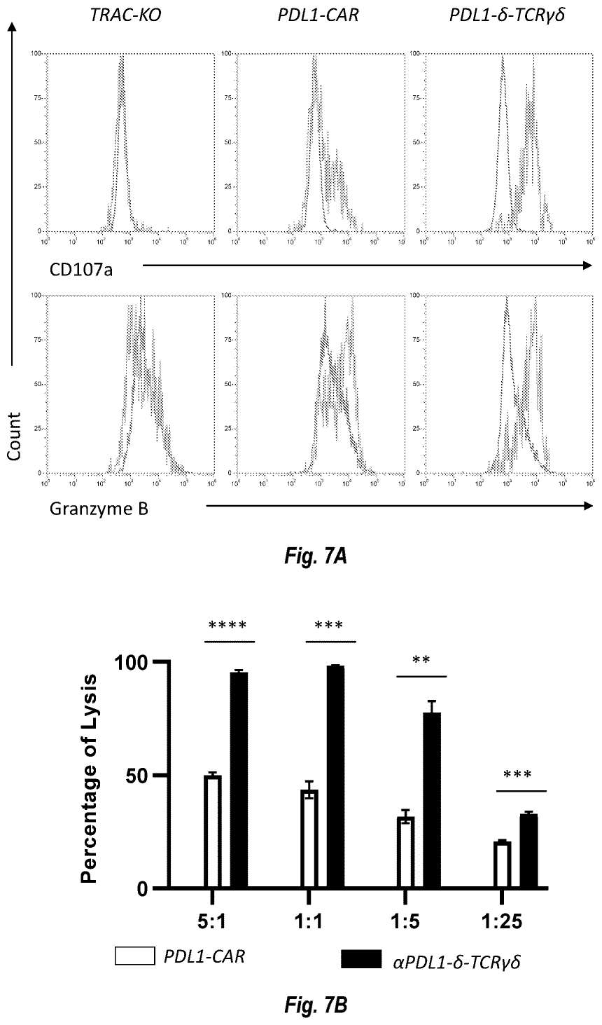

When co-cultured with PD-L1-expressing SK-MEL-5 melanoma cells, the PDL1 δ-TCRγδ-T cells demonstrated higher expression of the degranulation markers CD107a (upper panels) and granzyme B (lower panels), as assessed by flow cytometry, than PD-L1 CAR-T cells (FIG. 7A). FIG. 7B shows the results of cytotoxicity assays with varying ratios of effectors to SK-MEL-5 target cells. In these cytotoxicity assays the PD-L1 δ-TCRγδ-T cells significantly outperformed the PD-L1 CAR-T cells at all effector: target ratios.

### Example 11. In Vivo Anti-Tumor Efficacy of PD-L1-δ-γδTCR-T Cells in Lung Carcinoma, Melanoma, and Breast Cancer Models

To investigate whether αPDL1-δ-TCRγδ-T cells would show robust anti-tumor activity in vivo their ability to reduce tumor growth in a non-small cell lung cancer (NSCLC) xenograft mouse model was tested. Seven-week-old female NSG mice were purchased from The Jackson Laboratory, and 3×10⁶ A549 tumor cells mixed with Matrigel® (solubilized basement membrane matrix) were administered subcutaneously on the dorsal flank of each mouse. Mice with established tumors were randomly divided into different treatment groups (n=10) when xenograft tumor reached about 200 mm³. TRAC KO T cells, PDL1 CAR-T cells, and αPDL1-δ-TCRγδ-T cells were separately injected intravenously via tail vein (i.v.) to tumor bearing mice.

For the melanoma xenograft model, 4×10⁶ SK-MEL-5 tumor cells mixed with Matrigel® were administered subcutaneously on the dorsal flank of the mouse. Tumor grafted mice were divided into groups randomly when tumor reached about 200 mm³. TRAC KO T cells, PDL1 CAR-T cells, or αPDL1-δ-TCRγδ-T cells were injected intravenously into tumor bearing mice.

For the breast cancer xenograft model, 1×10⁶ MDA-MB-231 tumor cells mixed with Matrigel® were administered subcutaneously on the dorsal flank of each mouse. Tumor grafted mice were divided into groups randomly when tumor reached about 200 mm³. TRAC KO T cells, PDL1 CAR-T cells, or αPDL1-δ-TCRγδ-T cells were injected i.v. into tumor bearing mice.

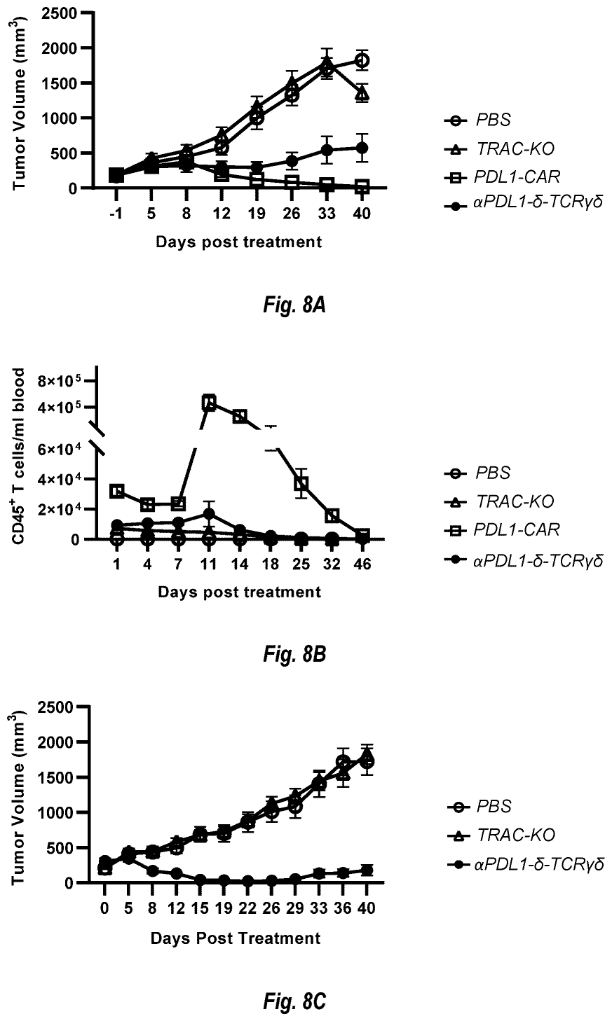

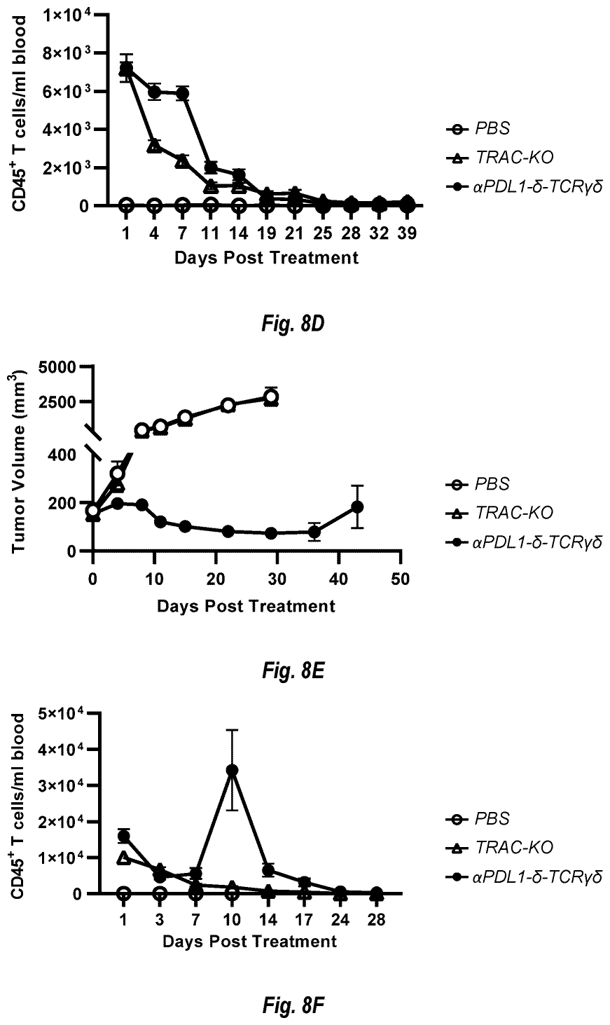

Tumor volume and body weight were measured twice a week (FIGS. 8A, 8C, and 8E). Additionally, to characterize the expansion of adoptive transferred T cells in vivo, the human CD45+ cell populations were monitored by analyzing the peripheral blood samples taken at the indicated time points (FIGS. 8B, 8D, and 8F).

FIG. 8A shows that treatment with the PD-L1 CAR-T or αPDL1-δ-TCRγ8-T cells suppressed tumor progression in mice bearing A549 tumors, with the PD-L1 CAR-T cells eradicating tumors by about 5 weeks. Flow cytometry data revealed a significant increase in CD45⁺ T cells from day 7 after adoptive CAR-T cell transfer, followed by a steady decline from day 11 to day 36, indicating a robust T cell expansion upon the encounter of tumor antigen and the subsequent contraction of T cells when tumor was cleared (FIG. 8B). In contrast to CAR-T, αPDL1-δ-TCRγδ-T cells exhibited limited T cell expansion in vivo (FIG. 8B).

In the melanoma xenograft model, the tumor grew rapidly in mice treated with PBS or TRAC-KO T cell controls, while αPDL1-δ-TCRγδ-T cells effectively repressed the tumor growth up to 40 days after infusion (FIG. 8C) and infused T cells in the peripheral blood were observed (FIG. 8D). In this case, transferred CAR-T cells failed to suppress the tumor progression (not shown), suggesting that αPDL1-δ-TCRγδ-T cells might be superior to CAR-T in certain solid tumors.

In the xenograft breast cancer model, αPDL1-δ-TCRγδ-T cells were able to suppress the tumor growth up to 36 days, with a small degree of T cell expansion occurring in the peripheral blood (FIG. 8E and FIG. 8F).

### Example 8: Degranulation and Expansion Assays Comparing αPDL1-δ-TCRγδ-T Cells and αPDL1-γ-TCRγδ-T Cells

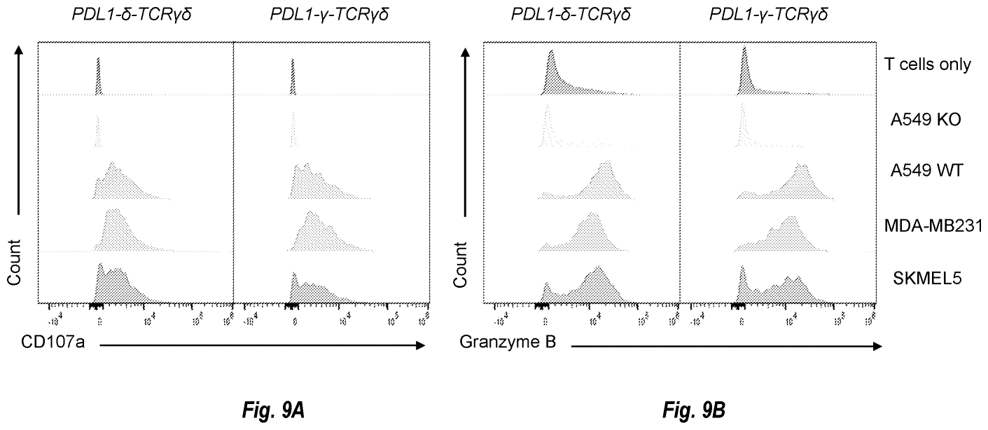

The expression of degranulation marker CD107a when cultured on various target cells was compared in cells expressing an engineered γδ receptor having an anti-PD-L1 scFv on the δ chain (αPDL1-δ-TCRγδ) and T cells expressing an engineered γδ receptor having an anti-PD-L1 scFv on the γ chain (αPDL1-γ-TCRγδ). FIG. 9A shows that both αPDL1-δ-TCRγδ-T cells and αPDL1-γ-TCRγδ-T cells increased expression of CD107a when cultured with A549 wild type cells, MDA-MB-231 cells, and SK-MEL5 cells in the presence of Brefeldin A, but not when cultured on PD-L1 knockout A549 cells or in the absence of target cells. FIG. 9B compares the expression of degranulation marker granzyme B by αPDL1-δ-TCRγδ-T cells and αPDL1-γ-TCRγδ-T cells after coculturing with target cells in the presence of Brefeldin A. In these assays, very little difference is seen between αPDL1-δ-TCRγδ-T cells and αPDL1-γ-TCRγδ-T cells in the expression of CD107a and granzyme B when cultured on tumor cells that express PD-L1 (A549 wild type, MDA-MB231, and SKMEL5 cells). The increased expression of these molecules was not observed when the cells expressing engineered receptors were cultured in the absence of tumor cells or with A549 PDL1 KO cells, demonstrating that the expression of degranulation markers was dependent on expression of PD-L1 by target cells.

To test expansion of the T cells expressing engineered αPD-L1 scFv-TCRs on PD-L1-expressing tumor cells, the GD102 (αPDL1-δ-TCRγδ expressing) T cells, GD109 (αPDL1-γ-TCRγδ expressing) T cells, and PDL1 CAR-T cells produced as in Examples 3 and 4 were cultured on A549 human lung carcinoma cells that express PD-L1. As a control, all three PD-L1 receptor-expressing cell types were cultured on A549 cells that had been knocked out for PD-L1 expression (A549KO cells; human CD274 (PD-L1) knockout A549 cell line; Abcam, Cambridge, UK). In addition, as further controls, ATCs and TRAC knockout cells were tested in identical assays as effector cells.

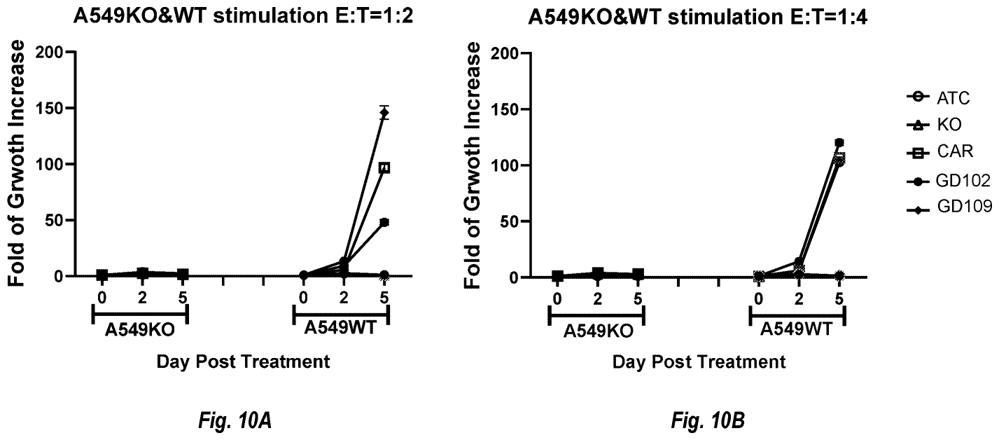

For the PD-L1-specific clonal expansion assay, A549 (engineered with GFP) and A549KO tumor cells were irradiated at 40GY to arrest cell cycle by using RS-2000 irradiator (Rad Source Technologies). A549 KO cells were prestained with CSFE (Thermo Fisher Scientific, Waltham, MA, USA) and then seeded in 48-well plate at 5×10⁵ cells/well. Either 2.5×10⁴ (effector to target (E:T) ratio of 1:2; FIG. 10A) or 1.25×10⁴ (E:T ratio of 1:4; FIG. 10B) of transfected GD102 T cells, GD109 T cells, or PDL1 CAR-T cells were added to each well of tumor cells and co-cultured at 37° C. for up to five days without IL-2. Cells were harvested and stained with APC-conjugated anti-AnnexinV and live/dead yellow (Thermo Fisher Scientific, Waltham, MA, USA) at days 0, 2, and 5 post tumor stimulation and analysed by flow cytometry for antigen-specific clonal expansion of T cells expressing engineered PD-L1 receptors.

FIG. 10A shows that T cells engineered to express each of the recombinant PD-L1 receptors (GD102 T cells, GD109 T cells, and PDL1 CAR-T cells) expanded on A549 wild type cells, but not on A549 PD-L1 KO cells, demonstrating that expansion of the T cells expressing recombinant receptors was PDL1 specific. At the 1:2 E:T ratio, the GD109 αPDL1-γ-TCRγδ-T cells expanded the most and the GD102 αPDL1-δ-TCRγδ-T cells expanded the least, while the PDL1 CAR-T cells demonstrated an intermediate level of expansion on PD-L1+ tumor cells relative to the αPDL1-TCRγδ-T cells. FIG. 10B shows that at the 1:4 E:T ratio, the GD102 T cells, GD109 T cells, and PD-L1 CAR-T cells all expanded equally well, showing greater than 100-fold expansion after seven days of co-culturing with PD-L1+ tumor cells, while showing no expansion on A549 PD-L1 knockout cells, demonstrating that expansion of the T cells with the engineered receptors was PD-L1-specific.

### Example 9. Cytotoxicity of αPDL1-8-TCRγδ-T Cells, αPDL1-γ-TCRγδ-T Cells, and PDL1 CAR-T Cells Toward A549 Human Lung Carcinoma Tumor Cells

Cytotoxicity assays using WT and KO A549 cells as targets were performed using the xCelligence Real-time Cell Analyzer Assay (Acea/Agilent). In an E-Plate View 96 (Acca Biosciences), A549 WT and A549 PD-L1 KO cells were seeded at 1×10⁴ cells per well and the plate was incubated overnight in the xCELLigence RTCA MP instrument (Acca Biosciences). After overnight incubation, the medium was changed to 100 μl fresh medium and 100 μl T cells were added to get E:T ratios of 5:1, 1:1, 1:5, and 1:25. For the ‘tumor only’ control 100 μl medium was added (the tumor lysis group was not included in this assay, since the highest E:T of GD109 demonstrated the reading of fully lysed tumor cells). The plate was returned to xCELLigence RTCA MP instrument (Acea Biosciences) for real-time impedance monitoring. Cytotoxicity was calculated using RTCA software pro (Acca Biosciences).

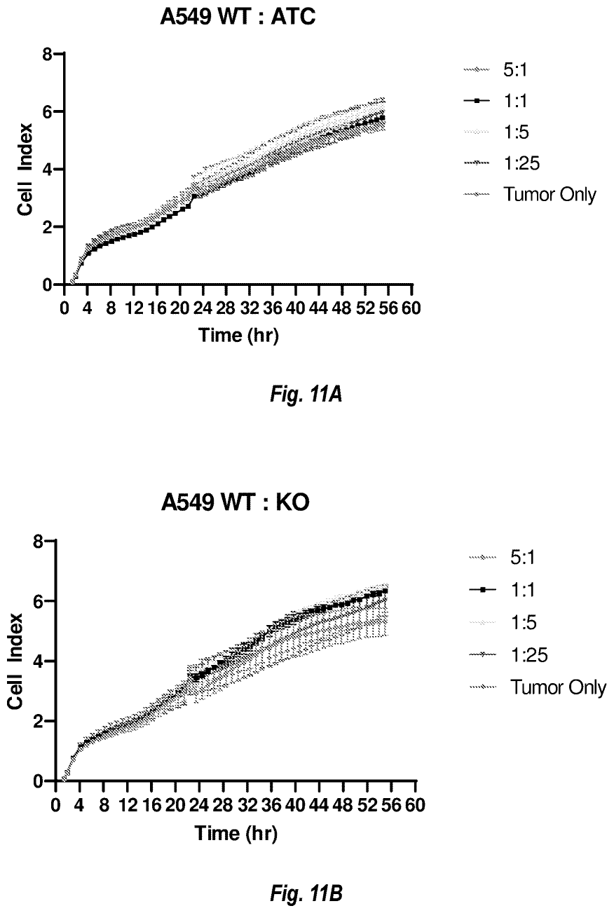

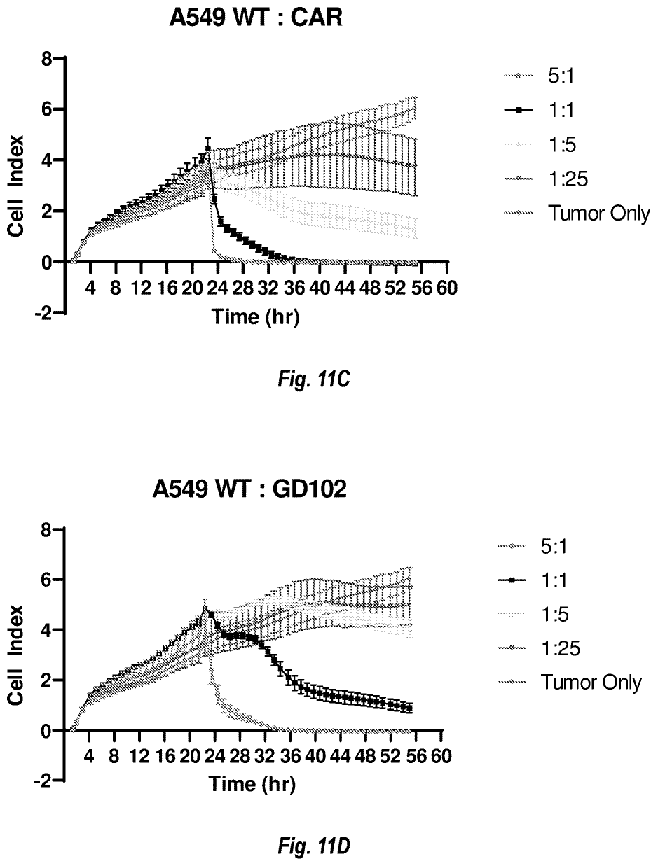

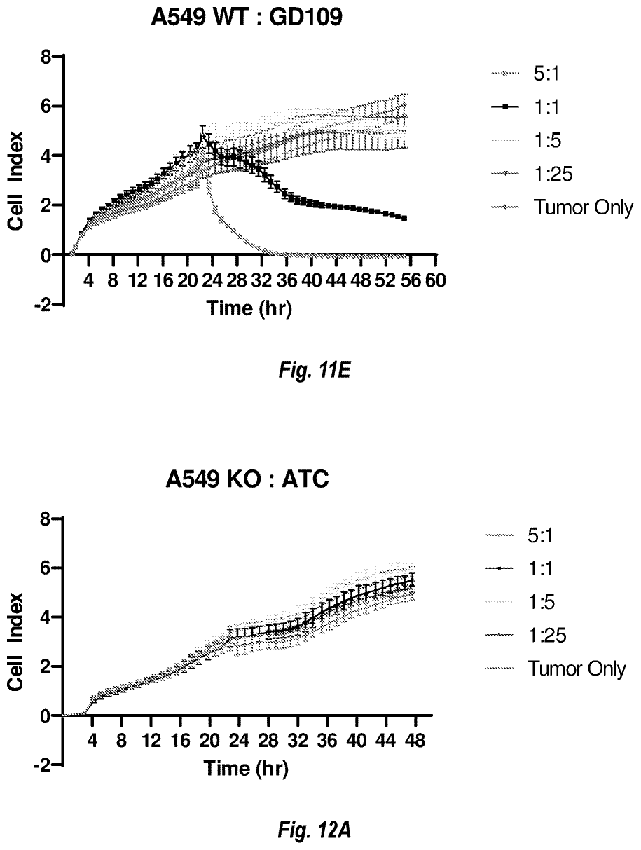

A549 WT cells were not killed by nontransfected T cells (ATCs) or TRAC knockout T cells, regardless of the E:T ratio (FIG. 11A and FIG. 11B). PDL1 CAR-T cells were highly effective at killing A549 WT cells at a 5:1 E:T beginning at about 24 hours, and were also effective at 1:1 E:T and somewhat less so at E:T of 1:5 (FIG. 11C). αPDL1-δ-TCRγδ-T cells and αPDL1-γ-TCRγδ-T cells at a 5:1 E:T were able to kill all of the target cells by 36 hours and at a 1:1 E:T, were able to kill most cells by about 48 hours into the assay (FIG. 11D and FIG. 11E).

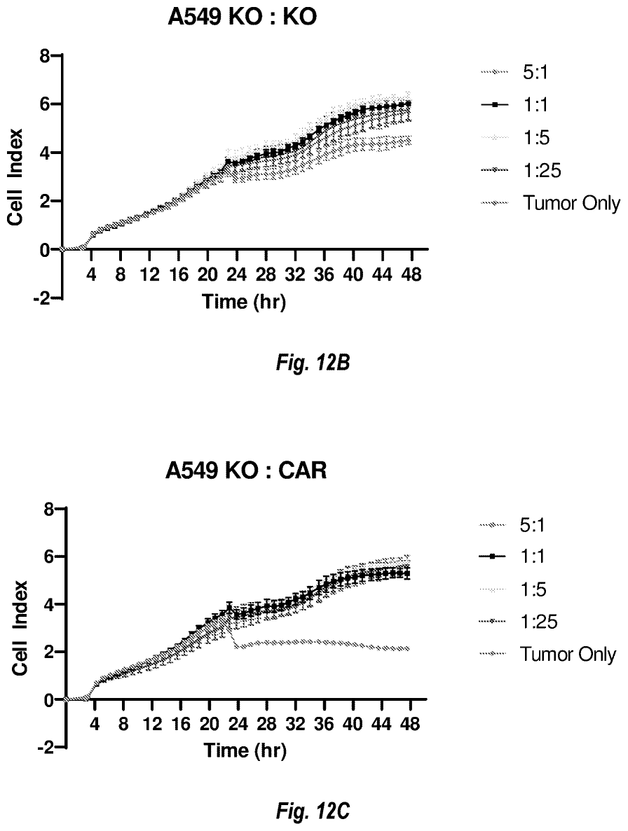

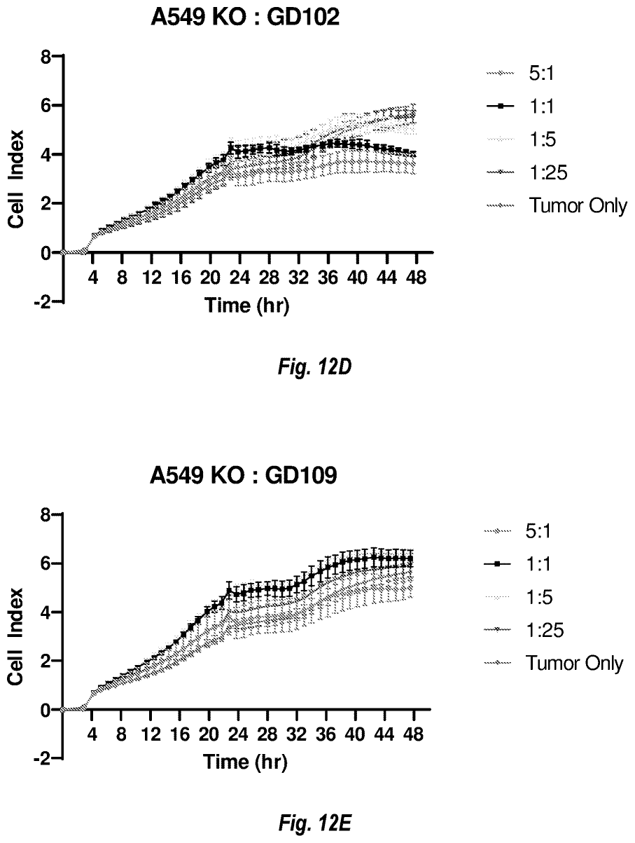

Results of the same assays using A549 PD-L1 knockout (KO) cells as targets are provided in FIGS. 12A-12E). In this case, there was no killing of A549 PD-L1 knockout targets by ATCs or TRAC knockout cells, regardless of E:T (FIG. 12A and FIG. 12B), nor were A549 PD-L1 knockout target cells killed by GD102 (αPD-L1-δ-TCRγδ expressing) T cells and GD109 (αPDL1-γ-TCRγδ expressing) T cells (FIG. 12D and FIG. 12E).

### Example 10. Cytotoxicity of αPDL1-δ-TCRγδ-T Cells, αPDL1-γ-TCRγδ-T Cells, and PDL1 CAR-T Cells Toward MDA-MB-231 Human Breast Cancer Tumor Cells

Cytotoxicity assays were also performed using MDA-MB 231 human breast cancer cells as targets were performed using the xCelligence Real-time Cell Analyzer Assay (Acca/Agilent). In E-Plate View 96 (Acea Biosciences) as described above in Example 9.

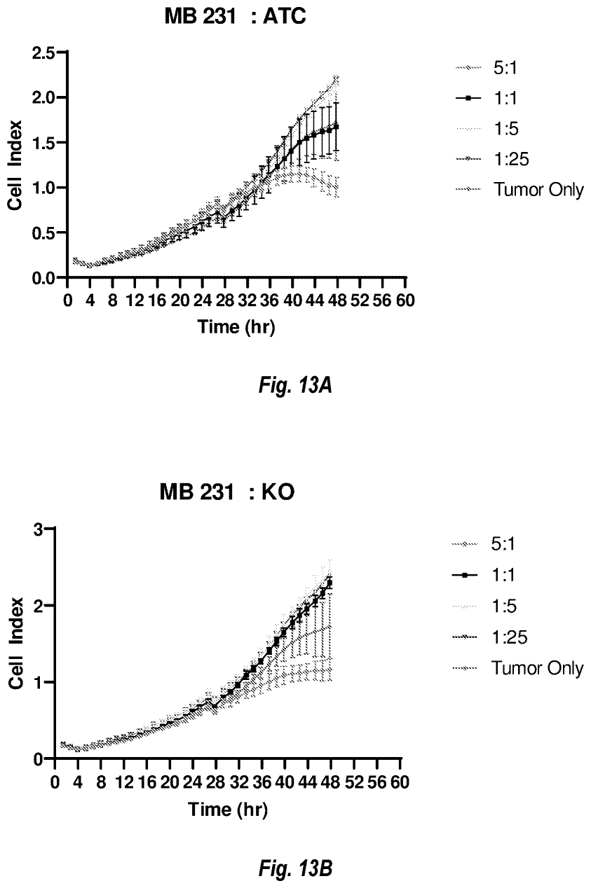

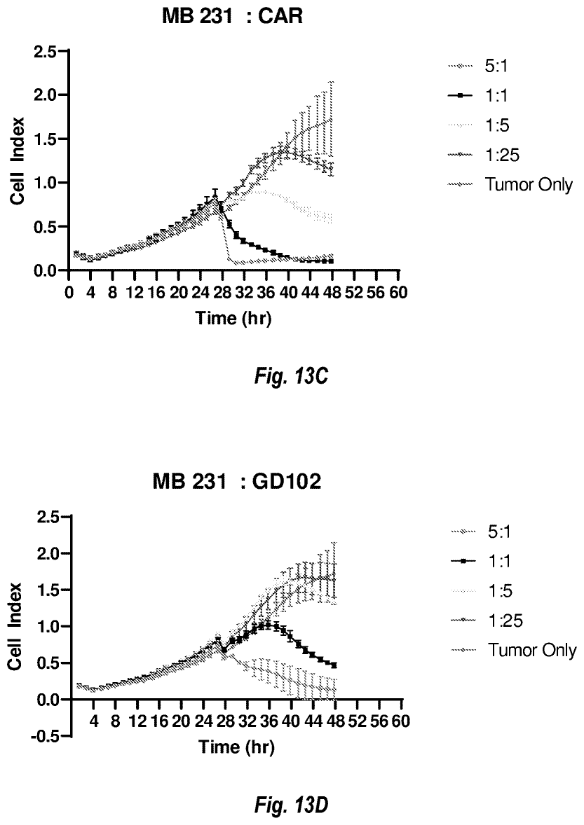

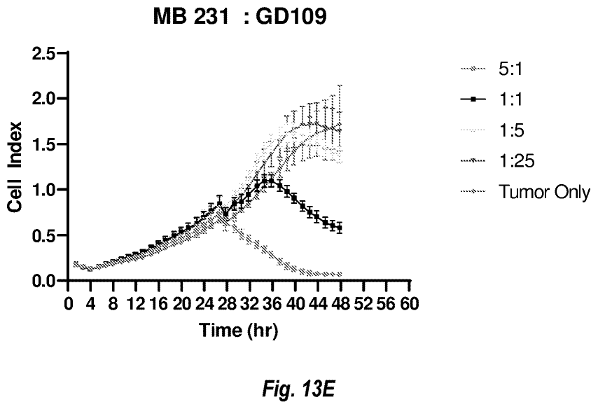

FIG. 13A and FIG. 13B show that MDA-MB 231 cells were not efficiently killed by nontransfected T cells (ATCs) or TRAC knockout T cells, although some killing was apparent over time at the highest E:T ratio (5:1). In contrast, all of the T cells expressing engineered PD-L1 receptors demonstrated cytotoxicity toward the MDA-MB 231 target cells. PD-L1 CAR-T cells were highly effective at killing A549 WT cells at a 5:1 E:T and a 1:1 E:T. and also demonstrated killing of the tumor cells at an E:T of 1:5 (FIG. 13C). αPDL1-δ-TCRγ-T cells and αPDL1-γ-TCRγδ-T cells demonstrated effective killing of the MDA-MB 231 tumor cells at 5:1 and 1:1 E:T ratios (FIG. 13D and FIG. 13E).

### Example 11. Cytokine Release Assays: αPDL1-δ-TCRγδ-T Cells and αPDL1-γ-TCRγδ-T Cells

Cytokine release assays were performed on engineered T cells co-cultured with A549 PD-L1 knockout cells and A549 wild type cells. Target cells were seeded in 96-well plate at 1×10⁵ cells/well. GD102 (αPDL1-δ-TCRγδ expressing) T cells, GD 109 (αPDL1-γ-TCRγδ expressing) T cells, or PD-L1 CAR-T cells, and as controls nontransfected T cells (ATCs) or TRAC knockout T cells, were added to each well of tumor cells at E:T ratios of 5:1, 1:1, 1:5, and 1:25. The cells were co-cultured at 37° C. overnight. Interferon gamma (IFNγ) and granulocyte macrophage colony stimulating factor (GM-CSF) were analyzed from the cell culture supernatants by using IFNγ and GM-CSF ELISA Kits (Thermo Fisher Scientific, Waltham, MA, USA).

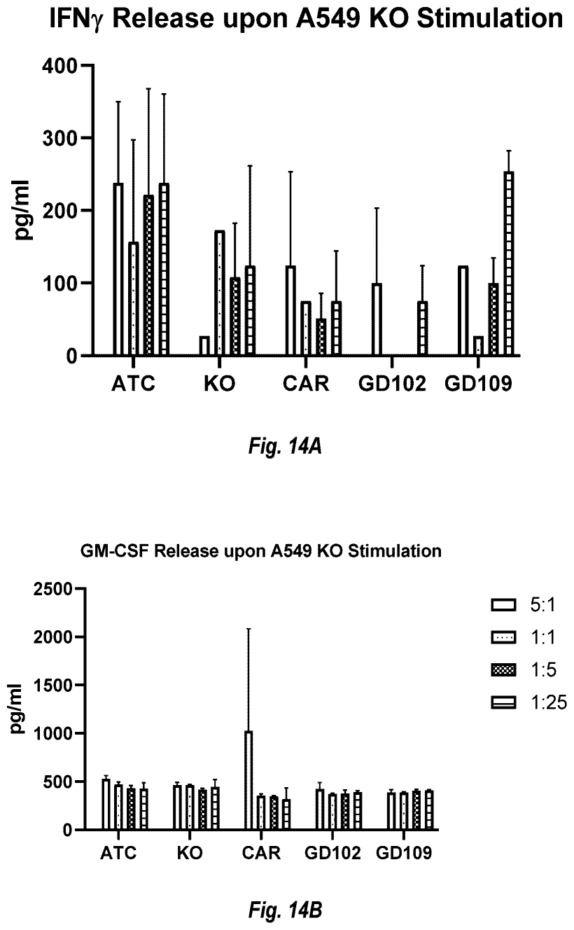

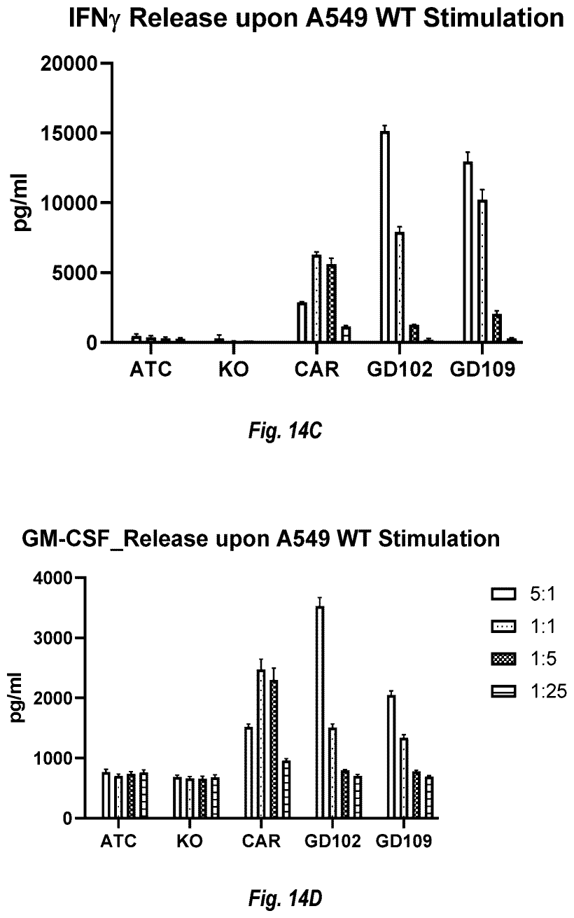

FIG. 14A shows the amount of IFNγ released by the T cells co-cultured with PD-L1 knockout tumor cells. Although the amount detected in the supernatants varied, overall the amounts detected were quite low, with none reaching more than about 250 μg/ml, when T cells were cultured with A549 wild type cells, the cells expressing engineered PD-L1 receptors (αPDL1-δ-TCRγδ (GD102), αPDL1-γ-TCRγδ (GD109), and PD-L1 CAR) released much greater amounts of IFNγ, up to 15,000 μg/ml, while ATCs and TRAC knockout cells released very small amounts of the cytokine (FIG. 14C).

The release of GM-CSF was also low for all co-cultures that included PD-L1 knockout A549 target cells, in almost all cases not exceeding 500 μg/ml (FIG. 14B). When T cells were co-cultured with wild type A549 cells, there was a much greater amount of GM-CSF produced by T cells expressing engineered PD-L1 receptors (FIG. 14D) The GD102 and GD109 cells stimulated with A549 wild type cells produced somewhat less GM-CSF than the CAR-T cells, except at the highest effector: target ratio of 5:1.

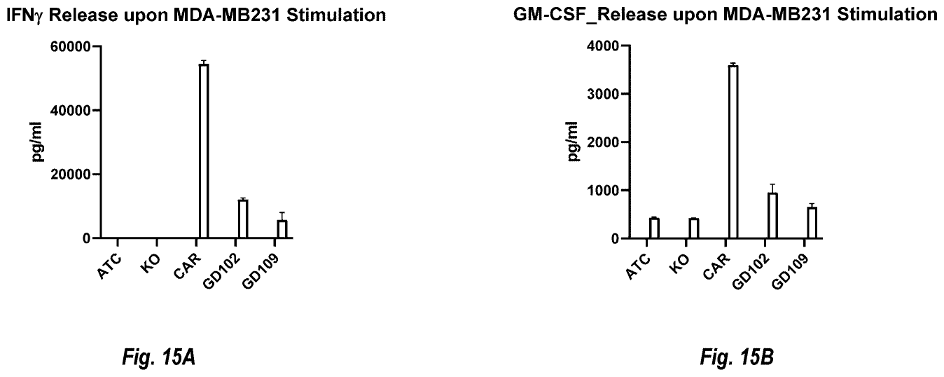

Cytokine release assays were also performed using MDA-MB-231 cells as targets. The MDA-MB-231 cells were co-cultured with T cells overnight at a 1:1 ratio. Cytokine release into supernatant was evaluated by ELISA. FIG. 15A shows that all of the T cells expressing engineered PD-L1 receptors (GD102 PD-L1 scFv-γδTCR, GD109 PD-L1 scFv-γδTCR, and PD-L1 CAR) released IFNγ in response to co-culture with MDA-MB-231 tumor cells, whereas none was detected from the ATC and TRAC knockout T cell co-culture supernatants. FIG. 15B shows that all of the T cells expressing engineered PD-L1 receptors (GD102, GD109, and PD-L1 CAR) released a greater amount of GM-CSF in response to co-culture with MDA-MB-231 tumor cells than was detected from the co-cultures with the ATCs and TRAC knockout T cells. T cells expressing either the GD102 (αPDL1-δ-TCRγδ) or the GD109 (αPDL1-γ-TCRγδ) engineered y& receptors expressed significantly less of both cytokines (IFNγ and GM-CSF) in co-culture with target cells than was expressed by PD-L1 CAR-T cells.

### Example 12. In Vivo Anti-Tumor Efficacy of αPD-L1 scFv-TCRγδ Cells in a Lung Carcinoma Model

Tumoricidal activity of transgenic T cells expressing engineered αPD-L1 scFv-TCRγδ T cells was investigated in a lung carcinoma mouse model in which the mice were injected with A549 human lung carcinoma cells.

Seven to nine week old female NSG mice were used for the study, with 10 mice per treatment group, where the groups included tumor-injected mice to be treated with: PBS only; TCR knockout (KO) cells, αPDL1-δ-TCRγδ-T cells, and αPDL1-γ-TCRγδ-T cells.

The tumor cells were prepared by transfecting cells of lung carcinoma cell line A549 expressing luciferase and GFP genes (A549-5-FLuc). To establish tumors in the mice, a total of 3×10⁶ cells of A549-5-FLuc were mixed with Matrigel® and then injected subcutaneously into the dorsal surfaces.

When post-inoculation tumor volume reached approximately 200 mm³, a single treatment of transgenic T cells engineered to express either: 1) GD102 (αPDL1-δ-TCRγδ); 2) GD109 (αPDL1-γ-TCRγδ); or 3) no engineered receptor, no T cell receptor (TRAC knockout) was administered. The αPDL1-δ-TCRγδ-T cells and αPDL1-γ-TCRγδ-T cells were administered to separate treatment groups in doses of 5×10⁵, 1×10⁶, 5×10⁶, and 1×10⁷ scFv-γδTCR-positive cells. The T cells were administered to the mice via the tail vein in PBS. A further injected sample included PBS only (no cells).

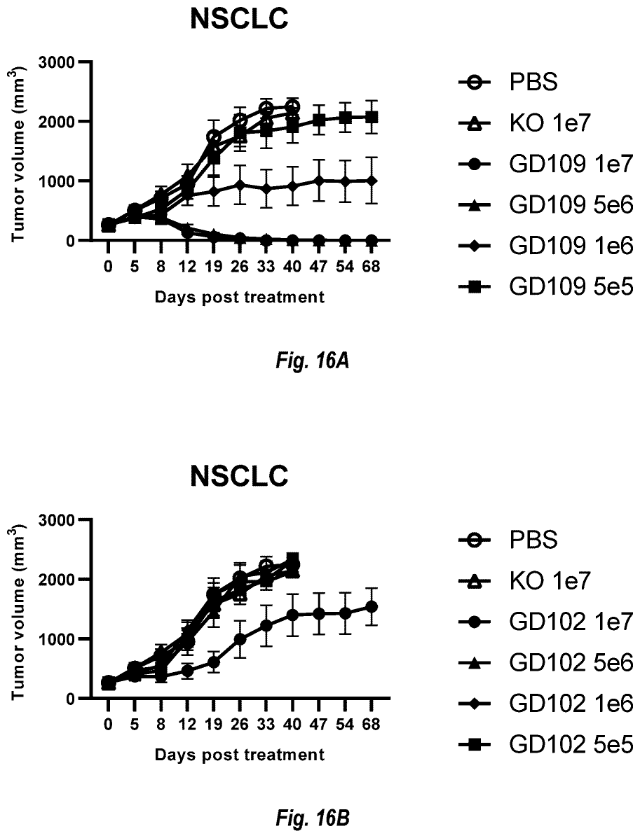

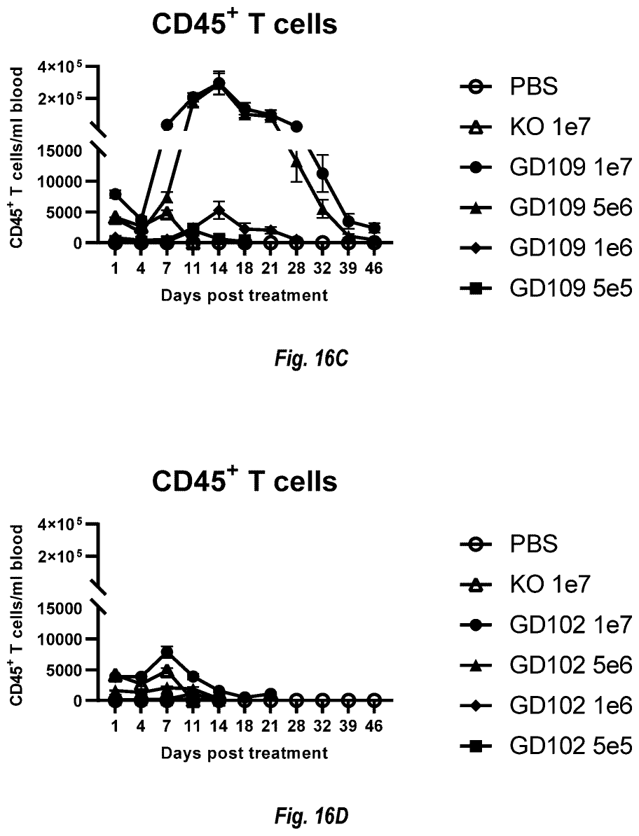

Tumors of tumor-bearing mice were measured with calipers on day 1 post treatment and twice per week thereafter. Mice were weighed weekly. The tumor volumes over the course of the experiment are shown in FIG. 16A (treatment with T cells expressing the GD109 (αPDL1-γ-TCRγδ) construct) and 16B (treatment with T cells expressing the GD102 (αPDL1-δ-TCRγδ) construct). In FIG. 16A, it can be seen that treatment with GD109 (αPDL1-γ-TCRγδ) T cells resulted in a dose-dependent suppression of tumor growth, and doses of 5×10⁶ and 1×10⁷ completely eradicated tumor by Day 33. The results with GD102 T cells were not as striking, with the highest dose (1×10⁷ cells) demonstrating reduction in tumor growth with respect to controls (FIG. 16B). FIG. 16C shows that the human T cells were detected in mouse blood for over 5 weeks after treatment when mice were treated with 5×10⁶ and 1×10⁷ GD109 T cells, while the transplanted GD102 T cells declined sharply in number beginning one week after treatment (FIG. 16D).

To study cell trafficking, subcutaneous tumors were removed on days 2, 4, and 7 post-treatment and numbers of CD45+ T cells in tumor tissue were analyzed by flow cytometry. FIG. 17A shows that γ-TCRγδ-T cells successfully infiltrated tumors 7 days after infusion, which coincided with the shrinkage of tumor mass observed from day 8 post treatment (FIG. 16A). In contrast, control T cells (TRAC knockout cells) failed to infiltrate into the tumor (FIG. 17A).

To observe infiltration of tumor by T cells in the treated mice, tumor tissues were removed and fixed with 10% formalin solution, and fixed tissue blocks were paraffin-embedded and sectioned. Slides were deparaffinized, rehydrated, and treated for antigen retrieval. Slides were blocked with normal goat serum for 20 min at room temperature and incubated with primary antibody against human CD45 overnight at 4° C. The slides were then incubated with biotinylated secondary antibody (Vector Laboratories) for 30 min at room temperature. Slides were incubated with VECTASTAIN Elite ABC Reagent peroxidase detection system and then incubated with peroxidase substrate solution for 10 min at room temperature until the desired intensity was attained. Slides were counterstained with hematoxylin QS (Vector Laboratories), dehydrated, mounted, and visualized using an Olympus CX33 Digital Microscope. FIG. 17B shows the striking contrast in T cell staining at Day 7 post-treatment between tumor in mice receiving control (TRAC KO) T cells and T cells expressing the recombinant αPDL1-γ-TCRγδ receptor, demonstrating infiltration of the tumor with infused αPDL1-Y-TCRγδ-T cells.

### Example 13. Anti-Tumor Efficacy of PD-L1 scFv-γδTCR Cells in Rechallenge with Lung Carcinoma Cells

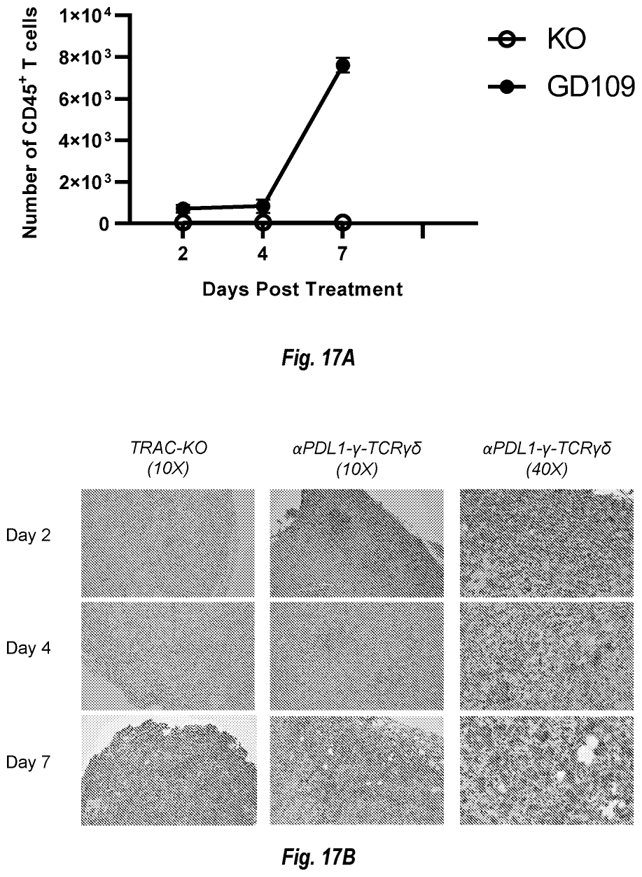

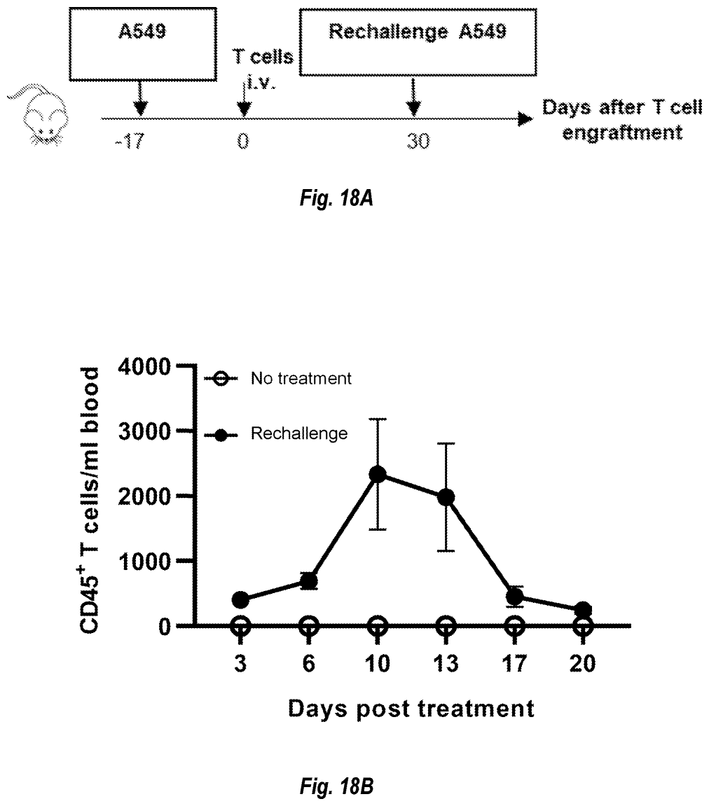

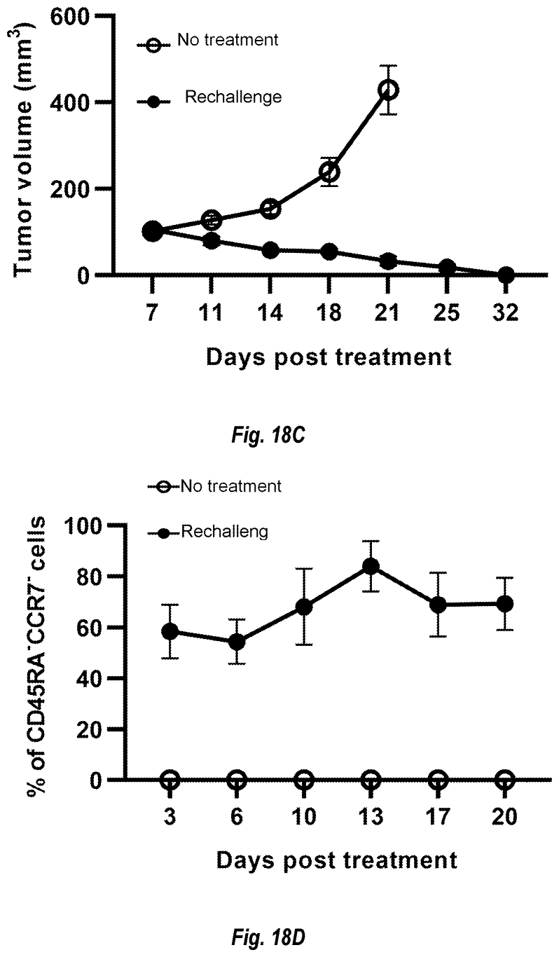

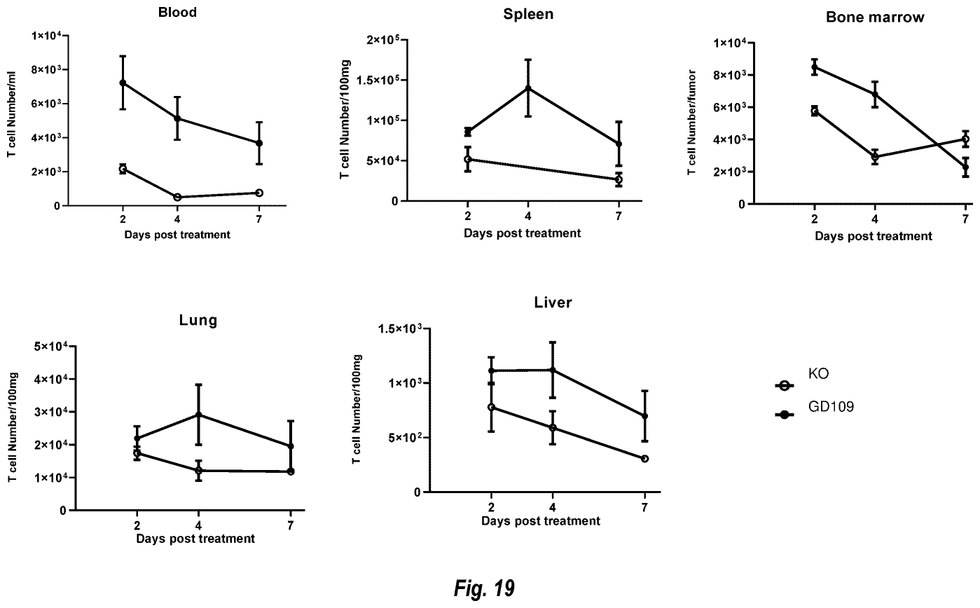

FIG. 18A shows the scheme of tumor re-challenge in the mice that were tumor free for five weeks after being treated with 1×10⁷ (GD109 αPDL1-γ-TCRγδ) T cells (Example 12). These mice were able to effectively eradicate tumor cells of a second inoculation (FIG. 18A) and showed proliferation of the T cells post rechallenge (FIG. 18C). The CD45⁺ cells underwent a rapid surge in numbers after re-encountering newly injected tumor cells, followed by the subsequent drop in the peripheral blood (FIG. 17B). Further analysis of the blood T cells by flow cytometry revealed that they exhibited effector/memory phenotype (FIG. 17D). FIG. 19 shows numbers of cells found in blood, spleen, bone marrow, lung, and liver of the re-challenged animals, demonstrating that in all cases, infiltration of T cells into tissues was found in animals that had received T cells expressing the recombinant αPD-L1-γ-TCRγδ receptors.

### Example 14. Transfection of T Cells with Constructs Encoding Single Engineered TCR Subunit Polypeptides

In further experiments, to better understand the assembly and functionality of TCR complexes that included engineered TCR subunits, constructs encoding only an engineered scFv-TCRδ polypeptide or only an engineered scFv-TCRγ polypeptide were independently transfected into isolated T cells.

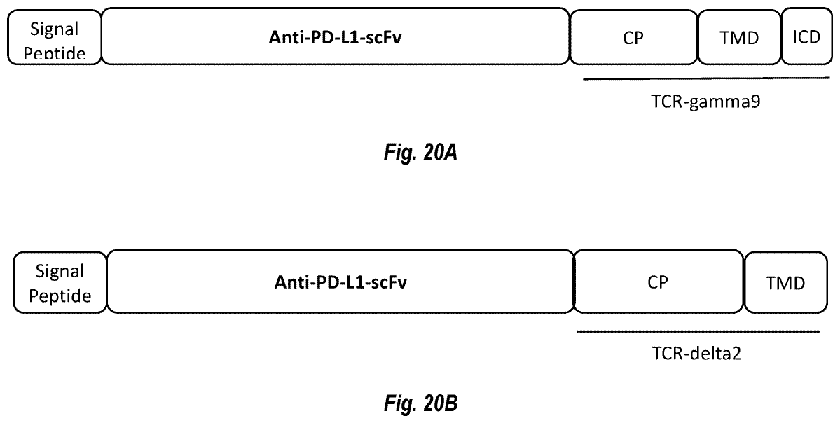

A first nucleic acid construct was generated that included a sequence encoding the αPDL1-TCRγ engineered subunit as described in Example 3 (SEQ ID NO:24; FIG. 20A). In this construct that encoded a single polypeptide, an anti-PD-L1 scFv antibody (SEQ ID NO:4) was followed by the extracellular connecting peptide of the TCRγ9 subunit (SEQ ID NO:9), the TCRγ9 subunit transmembrane domain (SEQ ID NO:10) and then the TCRγ9 subunit intracellular domain (SEQ ID NO:11), with a sequence encoding a signal peptide (SEQ ID NO:8) positioned at the N-terminus of the encoded precursor protein. The αPDL1-TCRγ construct (SEQ ID NO:24) was cloned in a vector downstream of a JeT promoter (SEQ ID NO:35).

A second nucleic acid construct was generated that also included a sequence encoding the αPDL1-TCRδ engineered subunit described in Example 3 (FIG. 20B). The same anti-PD-L1 scFv antibody (SEQ ID NO:4) was followed by the extracellular connecting peptide of the TCRδ2 subunit (SEQ ID NO:14) and then the TCR 82 subunit transmembrane domain (SEQ ID NO:15), with a sequence encoding a signal peptide (SEQ ID NO:13) positioned at the N-terminus of the encoded precursor protein. The αPDL1-TCRδ construct (SEQ ID NO:18) was also separately cloned in a vector downstream of a JeT promoter (SEQ ID NO:35).

Each construct, operably linked to the JeT promoter, was cloned in a vector between two flanking DNAs which were homology arms having sequences of the TRAC locus (SEQ ID NO:37 and SEQ ID NO:38). As described in Example 4, primers having homology to regions of the flanking sequences (SEQ ID NO:40 and SEQ ID NO:41) were used to generate donor fragments having homology arms of 171 bp and 161 bp (SEQ ID NO:42 and SEQ ID NO:43).

T cells were isolated and activated as described in Examples 1 and 2, and transfected with an RNP that included a guide RNA targeting exon 1 of the TRAC gene and one of the donor DNAs (either PD-L1 scFv-TCRγ or PD-L1 scFv-TCRδ, operably linked to the JeT promoter) as described in Example 4.

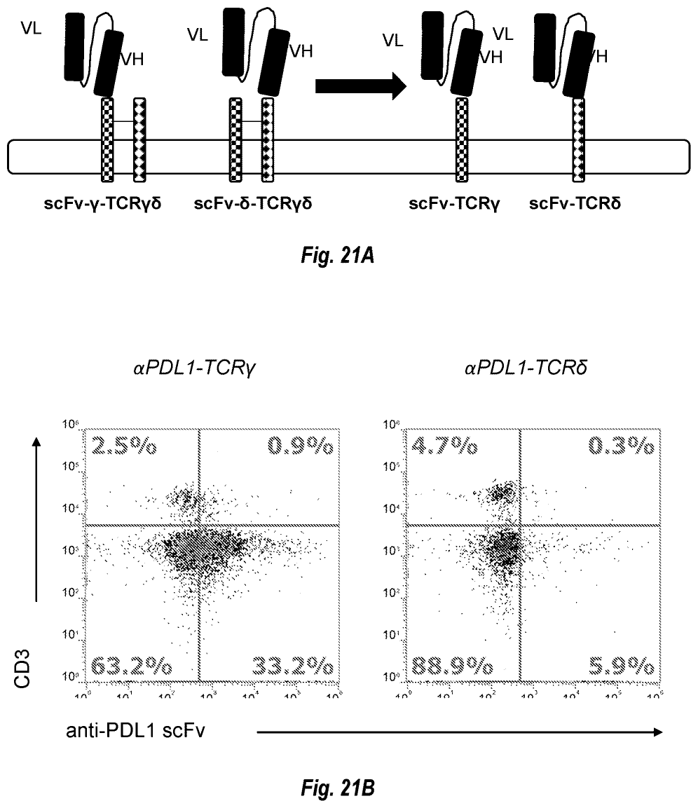

A schematic of scFv-TCRγ and scFv-TCRδ receptors that included single engineered polypeptide chains is compared with expressed scFv-γ-TCRγδ and scFv-8-TCRγδ receptors that included two engineered polypeptide chains as they would be expressed in cell membranes is provided in FIG. 21A. FIG. 21B provides the results of flow cytometry of cell populations 7 days after being independently transfected with the αPDL1-TCRγ construct and the αPDL1-TCRδ construct. Approximately 33% of the cell population transfected with the αPDL1-TCRγ construct expressed the construct while not expressing CD38, where essentially none of the cells that expressed the αPDL1-TCRγ construct expressed CD3ε. In contrast, the αPDL1-TCRδ construct was not stably expressed by the population transfected with this construct, with the transfected cells exhibiting neither the αPDL1-TCRδ polypeptide nor the CD3ε subunit.

### Example 15. Cytotoxicity of αPDL1-TCRγ-T Cells Toward A549 Human Lung Carcinoma Tumor Cells

Cytotoxicity assays were performed using T cells transfected with the construct encoding the αPDL1-TCRγ engineered polypeptide as effector cells. These effectors included the αPDL1-TCRγ construct but did not include a construct encoding any other engineered or non-engineered TCR subunits. Wild type (WT) and PD-L1 KO A549 cells were used as targets in these assays, where the PD-L1 gene was knocked out using a Cas9 RNP targeting the sequence of SEQ ID NO:39 in the TRAC gene. The assays were performed using the xCELLigence Real-time Cell Analyzer Assay (Acea/Agilent). In E-Plate View 96 (Acea Biosciences), A549 WT and A549 PD-L1 KO cells were seeded at 1×10⁴ cells per well and the plate was incubated overnight in the xCELLigence RTCA MP instrument (Acca Biosciences). After overnight incubation, the medium was changed to 100 μl fresh medium and 100 μl T cells were added to get E:T ratios of 5:1, 1:1, 1:5, and 1:25. For tumor only control 100 μl medium was added and for full lysis control 100 μl medium containing 0.5% Triton X-100 was added. The plate was returned to xCELLigence RTCA MP instrument (Acea Biosciences) for real-time impedance monitoring. Cytotoxicity was calculated using RTCA software pro (Acea Biosciences).

FIG. 22A shows that A549 PD-L1 KO cells were not killed by the PD-L1 scFv-TCRγ expressing cells, regardless of the E:T ratio. When wild type A549 cells were used as targets however, the αPDL1-TCRγ expressing cells were highly cytotoxic, killing the tumor cells at a 5:1 E:T and 1:1 E:T beginning at about 24 hours (FIG. 22B). The αPDL1-TCRγ cells were also effective at 1:5 E:T.

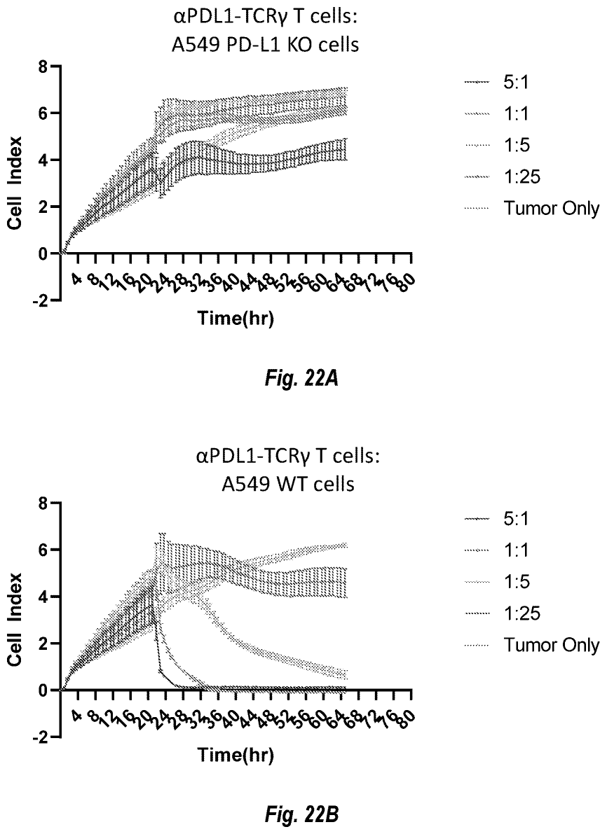

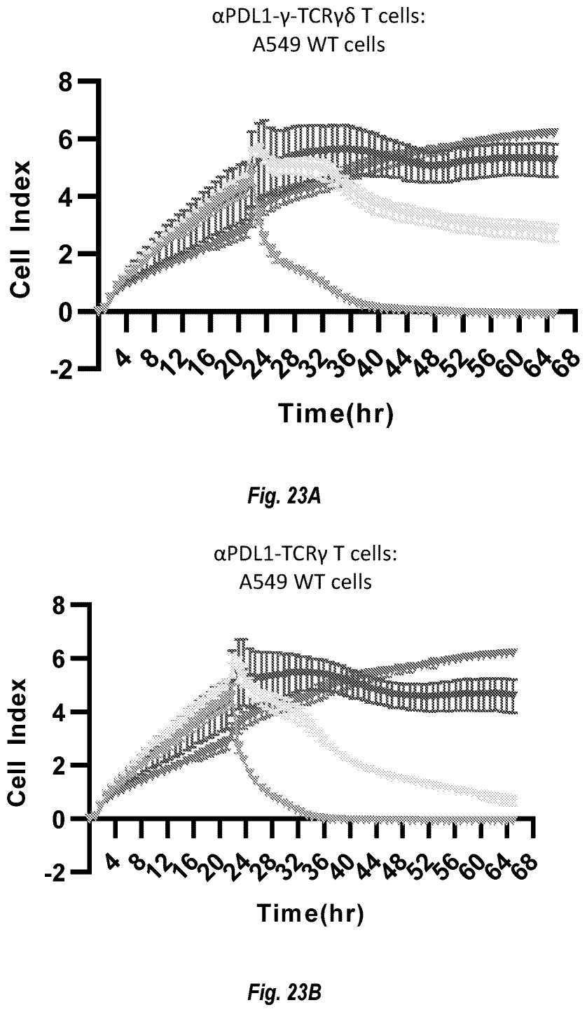

In separate assays, cytotoxicity of the αPDL1-TCRγ-T cells (transfected with a construct encoding a single polypeptide chain having the PDL1 scFv fused to the constant region of the TCRγ chain) was compared with cytotoxicity of αPDL1-γ-TCRγδ-T cells (transfected with a construct encoding a first polypeptide chain having the PDL1 scFv fused to the constant region of the TCRγ chain and a second polypeptide comprising a truncated TCRδ chain). The results of the RTCAs were performed as detailed above and provided remarkably similar results for the assays on A549 cells when αPDL1-γ-TCRγδ-T cells (FIG. 23A) and αPDL1-TCRγ-T cells (FIG. 23B) were used as effectors, where both types of engineered T cells showed dose-dependent killing.

### Example 16. In Vivo Anti-Tumor Efficacy of PD-L1 scFv-TCRγ-T Cells in a Lung Carcinoma Model

An in vivo study was performed to investigate the tumoricidal activity of T cells expressing the engineered PD-L1 scFv-TCRγ polypeptide in the absence of any other engineered receptor polypeptide. The study was performed essentially as described in Example 12, where seven to nine week old female NSG mice were injected with A549 human lung carcinoma cells and later treated with either 1×10⁷ T cells expressing either a single engineered TCR subunit with the αPDL1 scFv fused to γ chain sequences: αPDL1-TCRγ, or two engineered subunits: the αPDL1 scFv fused to γ chain sequences plus a truncated TCRδ chain (αPDL1-γ-TCRγδ) (10 mice per group).

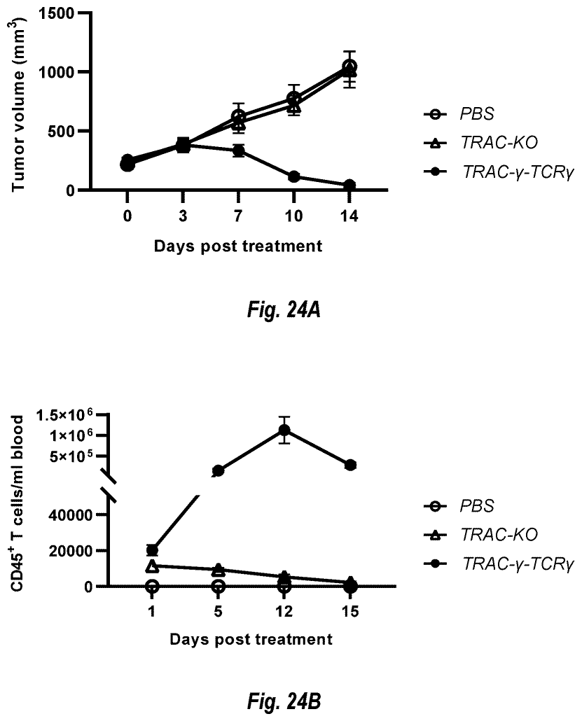

The tumor volumes over fourteen days are shown in FIG. 24A which shows that treatment with 1×10⁷ PD-L1 scFv-TCRγ-T cells essentially eradicated tumor by Day 14 post-treatment. FIG. 24B shows that the introduced human T cells expanded in the mouse during the experiment.

### Example 17. CD19 TCRγ Constructs

An αCD19 scFv-TCRγ construct was engineered to encode a single polypeptide analogous to the αPDL1-TCRγ polypeptide described in Example 14, except that instead of an anti-PDL1 single chain antibody (scFv), the αCD19 scFv-TCRγ polypeptide included an anti-CD19 single chain antibody (scFv) linked to sequences of the TCRγ chain. The anti-CD19 scFv (SEQ ID NO:60), based on the FMC63 anti-CD19 antibody, was followed by the extracellular connecting peptide of the TCRγ9 subunit (SEQ ID NO:9), which was followed by the TCRγ9 subunit transmembrane domain (SEQ ID NO:10) and then the TCRγ9 subunit intracellular domain (SEQ ID NO:11). The sequence encoding the mature αCD19-TCRγ polypeptide (SEQ ID NO:65) was preceded by a sequence encoding a signal peptide (SEQ ID NO:22) positioned at the N-terminus of the scFv moiety of the polypeptide. The nucleic acid sequence encoding the αCD19 scFv-TCRγ precursor polypeptide (SEQ ID NO:66) was cloned in a vector downstream of the JeT promoter (SEQ ID NO:35).

A CD19 CAR construct was also made. The CD19 CAR-ζFL (SEQ ID NO:29, encoded by SEQ ID NO:28) also included the anti CD19 scFv antibody (SEQ ID NO:4) that included the heavy chain variable region of the FMC63 antibody (SEQ ID NO:58) linked via a GS linker (SEQ ID NO:3) to the light chain variable region of the FMC63 antibody (SEQ ID NO:59). Following the light chain variable region was a CD8a hinge region (SEQ ID NO:30) followed by the CD8a transmembrane domain (SEQ ID NO:61) which was then followed by an intracellular domain that included the co-stimulatory domain of 4-1BB (SEQ ID NO:34) and the full-length signaling domain of CD35 (SEQ ID NO:33). The nucleic acid construct encoding the CD19 CAR-ζFL also included a sequence encoding a signal peptide (SEQ ID NO:22) positioned at the N-terminus of the protein. The entire CD19 CAR-ζFL construct was cloned in a vector downstream of a JeT promoter (SEQ ID NO:35).

The αCD19 scFv-TCRγ and CD19 CAR constructs were cloned between homology arms homologous to sequences of the TRAC gene and were separately transfected into primary human T cells from two different donors essentially according to methods described in Example 4.

### Example 18. Expansion of αCD19 scFv-TCRγ-T Cells on Target Cells

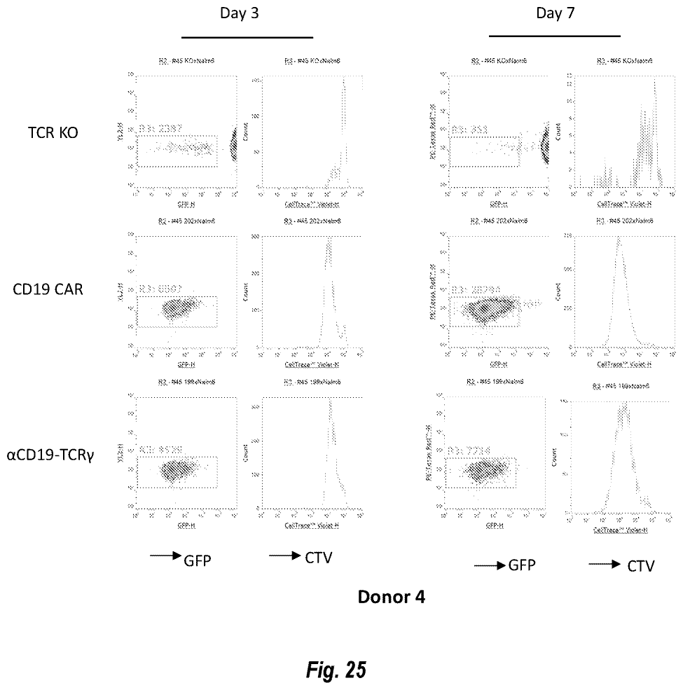

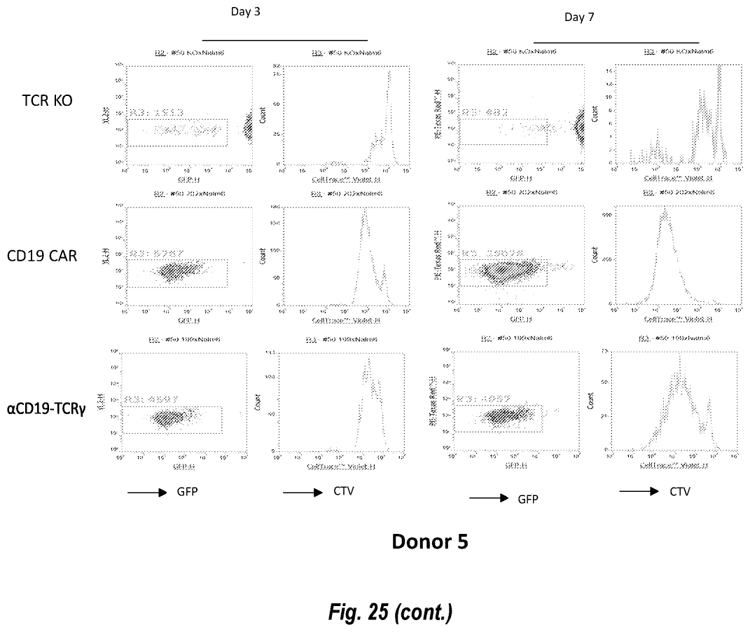

To test the ability of the αCD19 scFv-TCRγ-T cells to expand on target tumor cells, a population of CD19 positive Nalm-6 tumor cells carrying the GFP gene were plated at 5×10⁵ cells/well in wells of a 48-well plate. αCD19-TCRγ-T cells, CD19 CAR-T cells, or TCR knockout (KO) T cells that had been loaded with CTV dye were added to each well of tumor cells at a E:T ratio of 1:1 and the cells were co-cultured at 37° C. for up to seven days without IL-2. Cells were then analysed by flow cytometry by gating for GFP-negative cells and assessing CTV intensity. FIG. 25 shows that relative to the control TCR knockout cells, αCD19 scFv-TCRγ-T cells of Donor 4 had less of the CTV dye, indicating expansion on the target Nalm-6 cells, although to a slightly lesser degree than CD19 CAR-T cells. Comparison αCD19 scFv-TCRγ-T cells of Donor 5 with TCR knockout T cells of Donor 5 also showed the αCD19 scFv-TCRγ-T cells expanded on the CD19+ Nalm-6 cells.

### Example 19. Cytotoxicity Assay and Cytokine Release Assays Using αCD19 scFv-TCRγ-T Cells

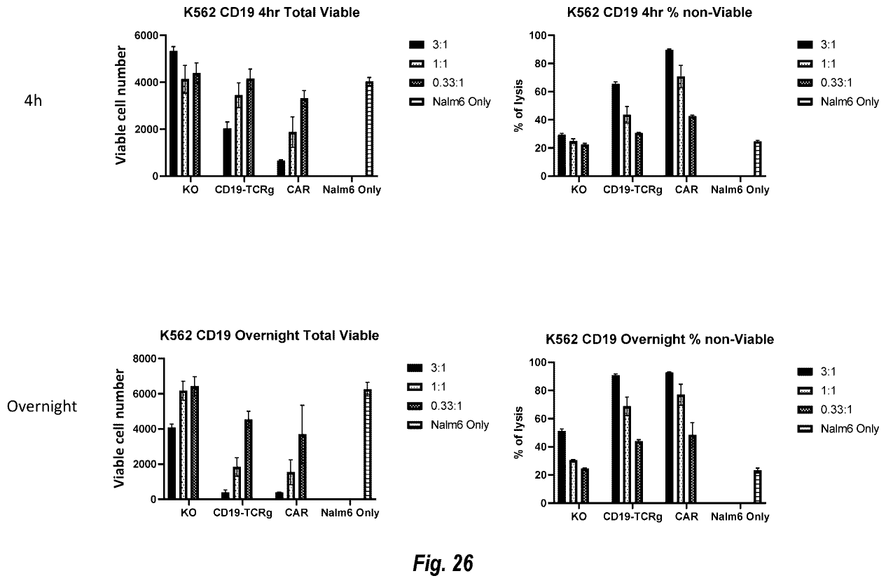

Cytotoxicity assays were also performed with K562 tumor cell lines engineered to express CD19 and GFP-Firefly luciferase (GFP-Fluc) as targets. Approximately 105 tumor cells were plated per well in a 96 well round-bottom plate and engineered αCD19 scFv-TCRγ-T cells (effector cells), CD19 CAR-T cells, or TRAC knockout (KO) T cells were added at E:T ratios of 3:1, 1:1, and 1:3. Cells were cocultured and after 4 hours and overnight coculturing the cells were stained with fixable viability dye (ThermoFisher), washed with Binding buffer, and then stained with Annexin V and analyzed by flow cytometry. Cytotoxicity was calculated as the percent of Annexin V-positive live/dead cells gated on the target cells. FIG. 26 shows that the engineered αCD19-TCRγ-T cells killed CD19-expressing K562 tumor cells in a dose-dependent manner. While the number of viable target cells after four hours of coculture was somewhat higher for the αCD19-TCRγ-T cells than for CD19-CAR-T cells, after 24 hours of coculturing the number of viable target cells for the TCRγ-T cells was highly similar to the number of viable cells in the CD19-CAR-T cell coculture (FIG. 26, left panels). This was reflected in the percent cytolysis data, which also showed the killing by αCD19-TCRγ-T cells caught up to that of CD19-CAR-T cells at 24 hours (FIG. 26, right panels).

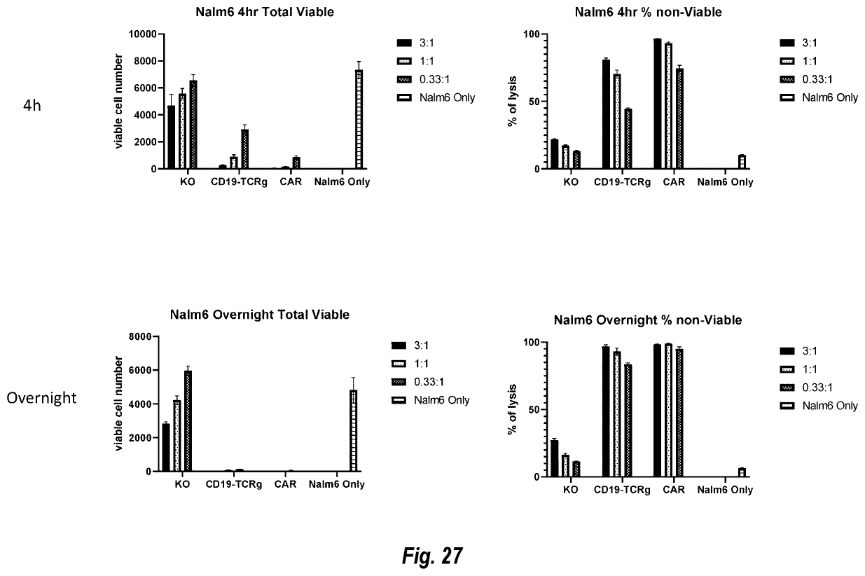

The same cytotoxicity assays were performed using GFP-expressing Nalm6 cells, cells of an acute lymphoblastic leukemia (ALL) cell line that express CD19, as targets. FIG. 27 (left panels) shows that the engineered αCD19-TCRγ-T cells killed Nalm6 tumor cells in a dose-dependent manner and were nearly as effective as αCD19-TCRγ-T cells. As was seen for K562 CD19+ targets (e.g., FIG. 26, left panels), after 24 hours of coculturing the number of viable target cells for the TCRγ-T cells was highly similar to the number of viable cells in the CD19-CAR-T cell coculture (FIG. 27, left panels). The percent cytolysis data was consistent with the viability data demonstrating the killing of Nalm6 leukemia cells by αCD19-TCRγ-T cells largely caught up to that of CD19-CAR-T cells by 24 hours (FIG. 27, right panels).

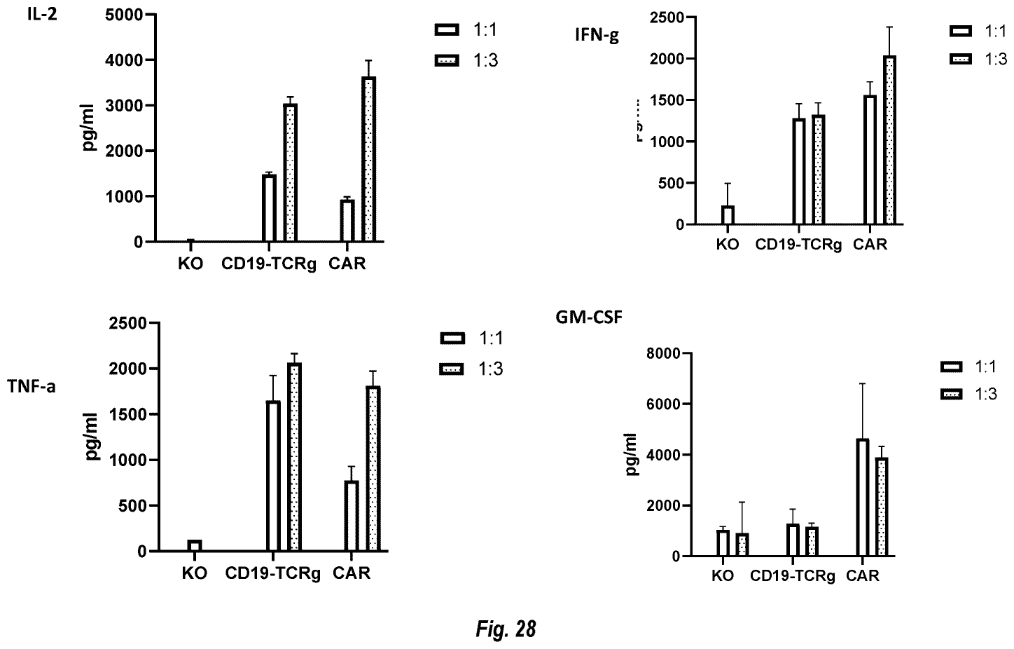

To measure secreted cytokines in cocultures of αCD19-TCRγ-T cells, CD19 CAR-T cells, and TRAC knockout (KO) T cells with Nalm6 target cells, 1×10⁵ effector cells were cocultured with 1×10⁵ tumor cells in 96-well plate at 37° C. overnight. Supernatants were collected and cytokines were measured using IFN-γ and GM-CSF ELISA kits (Thermo Fisher) and read by Cytation5 imaging reader. FIG. 28 shows that the αCD19-TCRγ-T cells produced somewhat less interleukin 2 (IL-2) and interferon gamma (IFN-g) and slightly more tumor necrosis factor alpha (TNF-α) than was produced by CD19 CAR-T cells. Notably, when co-cultured with Nalm6 target cells, the amount of GM-CSF produced by the αCD19-TCRγ-T cells was much lower than that produced by CAR-T cells.

### Example 20. In Vivo Anti-Tumor Efficacy of αCD19-TCRγ-T Cells in an ALL Model

Tumoricidal activity of transgenic T cells expressing engineered αCD19 scFv-TCRγ T cells was investigated in mice were injected with Nalm6 human leukemia cells.

Seven to nine week old female NSG mice were used for the study, with six mice per treatment group, where the groups included tumor-injected mice to be treated with: 1) 1×10⁷ TCR knockout (KO) T cells, 2) 1×10⁶ αCD19 scFv-TCRγ T cells, 3) 3×10⁶ αCD19 scFv-TCRγ T cells, 4) 1×10⁷ αCD19 scFv-TCRγ T cells, 5) 1×10⁶ CD19 CAR-T cells, 6) 3×10⁶ CD19 CAR-T cells, and 7) 1×10⁷ CD19 CAR-T cells.

To establish tumors in the mice, a total of 5×10⁵ of Nalm6 cells expressing luciferase and GFP genes (Nalm6-FLuc cells) were intravenously injected into each of the mice. Seven days later, on Day 0, mice were treated with T cells engineered to express either: 1) the GD102 αPDL1-δ-TCRγδ; 2) the GD109 αPDL1-γ-TCRγδ; or 3) no engineered receptor, no T cell receptor (TRAC knockout) was administered. The T cells were administered to the mice via the tail vein in PBS. Body weight and clinical behavior were monitored closely and blood sampling for detection of cytokines and introduced T cells was performed on Days 1, 3, 7, and 10 post-treatment. Imaging to monitor tumor size was performed twice a week.

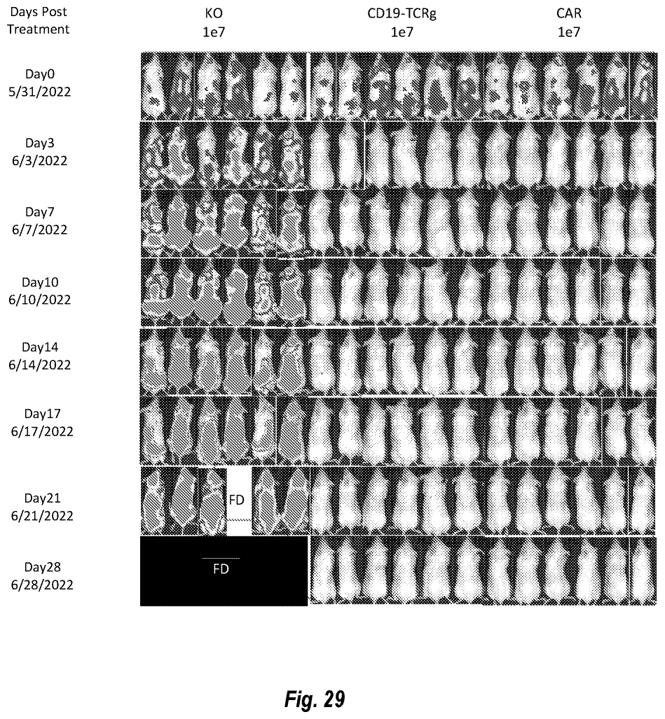

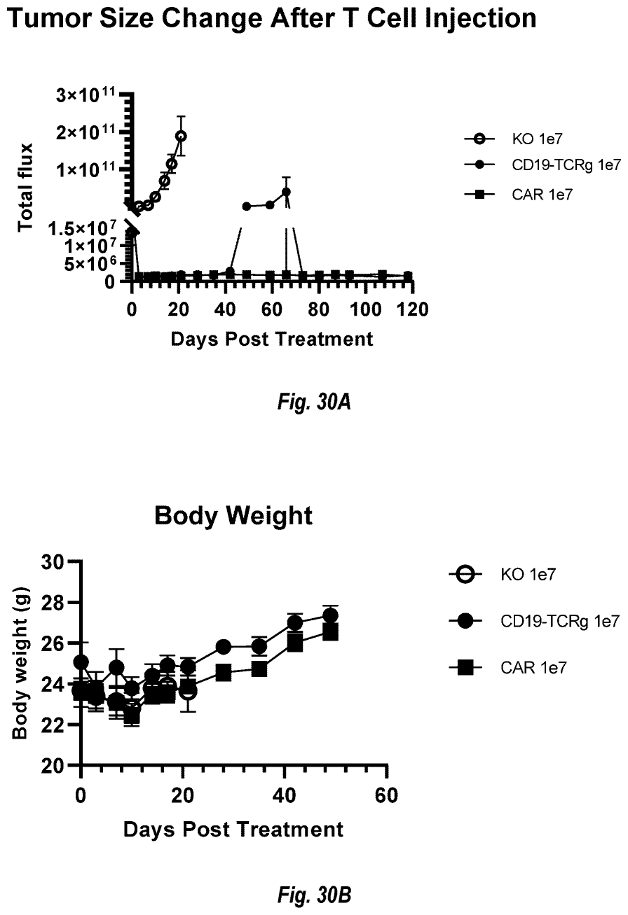

FIG. 29 provides the IVIS images of the treated mice through Day 28. Suppression of tumor growth can already be seen for both the αCD19-TCRγ-T cells and the CD19 CAR-T cell on Day 3 post-treatment. The CD19 CAR-T and the αCD19-TCRγ-T mice appear to be tumor free by Day 3. Recurrence of tumor occurred in one αCD19-TCRγ-T mouse on Day 42, with the remaining αCD19-TCRγ-T mice remaining tumor-free until the end of the study on Day 118. FIG. 30A provides the tumor volumes based on flux over the course of the experiment and FIG. 30B provides body weights over the same period.

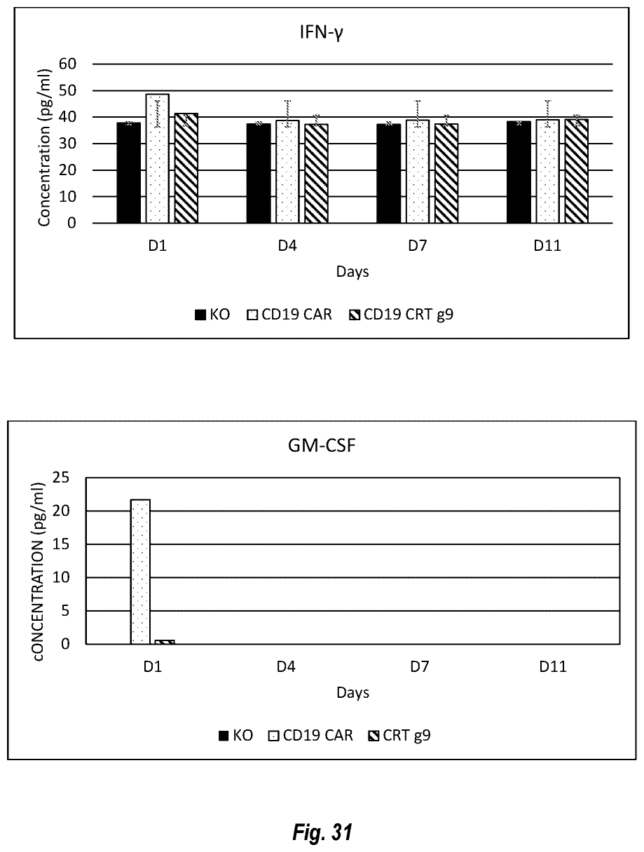

FIG. 31 provides the results of serum cytokine measurement performed on samples in the early stage of the study. All treated mice produced similar amounts of IFN-γ over the course of the 11 days; however, the difference in the amount of GM-CSF measured on Day 1 post-treatment was striking, where mice treated with αCD19-TCRγ-T cells at least ten-fold less GM-CSF than mice treated with CD19 CAR-T cells.

SEQUENCES
Protein
Artificial
SH1E2 PD-L1 antibody heavy chain variable region
SEQ ID NO: 1
EVQLVESGAEVKKPGSSVKVSCKASGGTFSSYAISWVRQAPGQGLEWMGGIIPIFGTANYAQ
KFQGRVTITADESTSTAYMELSSLRSEDTAVYYCARAPYYYYYMDVWGQGTTVTVSS
Protein
Artificial
SH1E2 PD-L1 antibody light chain variable region
SEQ ID NO: 2
QSALTQPASVSGSLGQSVTISCTGSSSDVGSYNLVSWYQQHPGKAPNLMIYDVSKRSGVSNRFSGSKS
GNTASLTISGLQAEDEADYYCSSYTGISTVVFGGGTKLTVL
Protein
Artificial
(G4S)₃ linker
SEQ ID NO: 3
GGGGSGGGGSGGGGS
Protein
Artificial
PD-L1 scFv
SH1E2 heavy chain-(G4S)₃ -SH1E2 light chain
SEQ ID NO: 4
EVQLVESGAEVKKPGSSVKVSCKASGGTFSSYAISWVRQAPGQGLEWMGGIIPIFGTANYAQKFQGRV
TITADESTSTAYMELSSLRSEDTAVYYCARAPYYYYYMDVWGQGTTVTVSSGGGGSGGGGSGGGGSQS
ALTQPASVSGSLGQSVTISCTGSSSDVGSYNLVSWYQQHPGKAPNLMIYDVSKRSGVSNRFSGSKSGN
TASLTISGLQAEDEADYYCSSYTGISTVVFGGGTKLTVL
Protein
Artificial
PD-L1 scFv
SH1E2 light chain-(G4S)₃ -SH1E2 heavy chain
SEQ ID NO: 5
QSALTQPASVSGSLGQSVTISCTGSSSDVGSYNLVSWYQQHPGKAPNLMIYDVSKRSGVSNRFSGSKS
GNTASLTISGLQAEDEADYYCSSYTGISTVVFGGGTKLTVLGGGGSGGGGSGGGGSEVQLVESGAEVK
KPGSSVKVSCKASGGTFSSYAISWVRQAPGQGLEWMGGIIPIFGTANYAQKFQGRVTITADESTSTAY
MELSSLRSEDTAVYYCARAPYYYYYMDVWGQGTTVTVSS
DNA
Artificial
Construct that includes sequence encoding Signal peptide-Truncated
gamma9 polypeptide-T2A sequence-Chimeric anti-PD-L1 ScFv/delta2
polypeptide
SEQ ID NO: 6
ATGTTGAGTTTGCTCCACACGTCAACATTGGCAGTCCTGGGGGCTCTGTGCGTGTACGGATTCCCGCC
CATCAAGACCGATGTGATCACAATGGACCCCAAGGACAACTGCAGCAAGGACGCCAACGATACCCTGC
TGCTGCAGCTGACCAACACCAGCGCCTACTACATGTATCTCCTGCTGCTGCTGAAGAGCGTGGTGTAC
TTCGCCATCATCACCTGCTGTCTGCTGCGGCGGACCGCCTTCTGCTGCAACGGCGAGAAGAGCGGCTC
TGGAGCTACTAACTTCAGCCTGCTGAAGCAGGCTGGAGACGTGGAGGAGAACCCTGGACCTATGTCCG
TCCCTACCCAGGTGCTGGGCCTGCTGCTGCTGTGGCTGACCGATGCTAGATGCGAAGTCCAACTGGTG
GAATCTGGAGCCGAGGTAAAGAAACCTGGCTCAAGTGTAAAAGTATCATGCAAAGCGTCCGGCGGCAC
GTTTTCAAGTTACGCCATAAGTTGGGTGAGACAAGCTCCCGGACAGGGACTTGAGTGGATGGGAGGAA
TTATACCTATTTTTGGGACTGCGAATTATGCACAAAAGTTCCAGGGAAGGGTTACTATCACAGCGGAC
GAGAGTACAAGTACAGCCTATATGGAGTTGTCTAGTCTCAGATCTGAGGACACTGCTGTCTATTATTG
TGCGCGAGCGCCTTACTATTACTACTATATGGACGTATGGGGCCAAGGCACTACGGTCACAGTAAGTT
CTGGCGGCGGCGGCAGCGGTGGCGGTGGCTCAGGTGGTGGTGGTTCTCAGAGTGCCCTTACTCAGCCT
GCCAGTGTATCCGGTTCACTTGGACAATCAGTTACAATATCTTGTACGGGCTCTTCATCCGACGTGGG
CAGTTATAATCTCGTTTCTTGGTATCAACAACACCCCGGCAAAGCACCCAATCTGATGATCTACGATG
TCAGTAAACGAAGCGGTGTCAGTAACAGGTTCTCAGGTTCAAAATCCGGCAATACCGCGAGTCTTACA
ATTTCCGGTTTGCAGGCGGAGGATGAGGCCGACTACTATTGTTCATCCTATACAGGTATTAGCACCGT
TGTGTTTGGCGGGGGCACTAAACTCACTGTTCTCGAGGTGAAAACCGACTCCACCGACCACGTGAAGC
CCAAAGAGACCGAGAACACCAAGCAGCCCAGCAAGAGCTGCCACAAGCCCAAGGCCATCGTGCACACC
GAGAAGGTGAACATGATGAGCCTGACCGTGCTGGGCCTGCGGATGCTGTTCGCCAAGACAGTGGCCGT
GAACTTCCTGCTGACCGCCAAGCTGTTCTTCCTG
Protein
Artificial
Precursor PD-L1 scFv-γδTCR polypeptide encoded by
SEQ ID NO: 7
MLSLLHTSTLAVLGALCVYGFPPIKTDVITMDPKDNCSKDANDTLLLQLTNTSAYYMYLLLL
LKSVVYFAIITCCLLRRTAFCCNGEKSGSGEGRGSLLTCGDVEENPGPMSVPTQVLGLLLLW
LTDARCEVQLVESGAEVKKPGSSVKVSCKASGGTFSSYAISWVRQAPGQGLEWMGGIIPIFG
TANYAQKFQGRVTITADESTSTAYMELSSLRSEDTAVYYCARAPYYYYYMDVWGQGTTVTVS
SGGGGSGGGGSGGGGSQSALTQPASVSGSLGQSVTISCTGSSSDVGSYNLVSWYQQHPGKAP
NLMIYDVSKRSGVSNRFSGSKSGNTASLTISGLQAEDEADYYCSSYTGISTVVFGGGTKLTV
LEVKTDSTDHVKPKETENTKQPSKSCHKPKAIVHTEKVNMMSLTVLGLRMLFAKTVAVNFLL
TAKLFFL
Protein
Artificial
Signal Peptide
SEQ ID NO: 8
MLSLLHTSTLAVLGALCVYGEP
Protein
Homo sapiens
TCR Gamma 9 chain extracellular connecting peptide
SEQ ID NO: 9
PIKTDVITMDPKDNCSKDANDTLLLQLTNTSA
Protein
Homo sapiens
TCR Gamma 9 chain transmembrane domain
SEQ ID NO: 10
YYMYLLLLLKSVVYFAIITCCLL
Protein
Homo sapiens
TCR Gamma 9 chain cytoplasmic domain
SEQ ID NO: 11
RRTAFCCNGEKS
Protein
Artificial
T2A self-cleaving sequence
SEQ ID NO: 12
GSGEGRGSLLTCGDVEENPGP
Protein
Artificial
Signal Peptide (Light chain IgG leader sequence)
SEQ ID NO: 13
MSVPTQVLGLLLLWLTDARC
Protein
Homo sapiens
TCRδ2 extracellular connecting peptide
SEQ ID NO: 14
EVKTDSTDHVKPKETENTKQPSKSCHKPKAIVHTEKVNMMSLTVLGLR
Protein
Homo sapiens
TCRδ2 transmembrane domain
SEQ ID NO: 15
MLFAKTVAVNELLTAKLFFL
Protein
Artificial
Precursor N-terminally truncated TCR Gamma 9 subunit
SEQ ID NO: 16
MLSLLHTSTLAVLGALCVYGFPPIKTDVITMDPKDNCSKDANDTLLLQLTNTSAYYMYLLLL
LKSVVYFAIITCCLLRRTAFCCNGEKS
Protein
Artificial
Mature N-terminally truncated TCR Gamma 9 subunit
SEQ ID NO: 17
PIKTDVITMDPKDNCSKDANDTLLLQLTNTSAYYMYLLLLLKSVVYFAIITCCLLRRTAFCC
NGEKS
Protein
Artificial
Precursor Chimeric anti-PD-L1 scFv-TCRδ subunit
SEQ ID NO: 18
MSVPTQVLGLLLLWLTDARCEVQLVESGAEVKKPGSSVKVSCKASGGTFSSYAISWVRQAPG
QGLEWMGGIIPIFGTANYAQKFQGRVTITADESTSTAYMELSSLRSEDTAVYYCARAPYYYY
YMDVWGQGTTVTVSSGGGGSGGGGSGGGGSQSALTQPASVSGSLGQSVTISCTGSSSDVGSY
NLVSWYQQHPGKAPNLMIYDVSKRSGVSNRFSGSKSGNTASLTISGLQAEDEADYYCSSYTG
ISTVVFGGGTKLTVLEVKTDSTDHVKPKETENTKQPSKSCHKPKAIVHTEKVNMMSLTVLGL
RMLFAKTVAVNFLLTAKLFFL
Protein
Artificial
Mature Chimeric anti-PD-L1 scFv-TCRδ subunit
SEQ ID NO: 19
EVQLVESGAEVKKPGSSVKVSCKASGGTFSSYAISWVRQAPGQGLEWMGGIIPIFGTANYAQKFQGRV
TITADESTSTAYMELSSLRSEDTAVYYCARAPYYYYYMDVWGQGTTVTVSSGGGGSGGGGSGGGGSQS
ALTQPASVSGSLGQSVTISCTGSSSDVGSYNLVSWYQQHPGKAPNLMIYDVSKRSGVSNRFSGSKSGN
TASLTISGLQAEDEADYYCSSYTGISTVVFGGGTKLTVLEVKTDSTDHVKPKETENTKQPSKSCHKPK
AIVHTEKVNMMSLTVLGLRMLFAKTVAVNFLLTAKLFFL
DNA
Artificial
Construct that includes sequence encoding Signal peptide - Chimeric
anti-PD-L1/ gamma9 polypeptide - T2A sequence - Signal peptide -
Truncated delta2 polypeptide
SEQ ID NO: 20
ATGGAGTGGTCCTGGGTGTTCCTGTTCTTTCTGTCCGTGACCACCGGTGTCCACTCTGAAGTCCAACT
GGTGGAATCTGGAGCCGAGGTAAAGAAACCTGGCTCAAGTGTAAAAGTATCATGCAAAGCGTCCGGCG
GCACGTTTTCAAGTTACGCCATAAGTTGGGTGAGACAAGCTCCCGGACAGGGACTTGAGTGGATGGGA
GGAATTATACCTATTTTTGGGACTGCGAATTATGCACAAAAGTTCCAGGGAAGGGTTACTATCACAGC
GGACGAGAGTACAAGTACAGCCTATATGGAGTTGTCTAGTCTCAGATCTGAGGACACTGCTGTCTATT
ATTGTGCGCGAGCGCCTTACTATTACTACTATATGGACGTATGGGGCCAAGGCACTACGGTCACAGTA
AGTTCTGGCGGCGGCGGCAGCGGTGGCGGTGGCTCAGGTGGTGGTGGTTCTCAGAGTGCCCTTACTCA
GCCTGCCAGTGTATCCGGTTCACTTGGACAATCAGTTACAATATCTTGTACGGGCTCTTCATCCGACG
TGGGCAGTTATAATCTCGTTTCTTGGTATCAACAACACCCCGGCAAAGCACCCAATCTGATGATCTAC
GATGTCAGTAAACGAAGCGGTGTCAGTAACAGGTTCTCAGGTTCAAAATCCGGCAATACCGCGAGTCT
TACAATTTCCGGTTTGCAGGCGGAGGATGAGGCCGACTACTATTGTTCATCCTATACAGGTATTAGCA
CCGTTGTGTTTGGCGGGGGCACTAAACTCACTGTTCTCCCCATCAAGACCGATGTGATCACAATGGAC
CCCAAGGACAACTGCAGCAAGGACGCCAACGATACCCTGCTGCTGCAGCTGACCAACACCAGCGCCTA
CTACATGTATCTCCTGCTGCTGCTGAAGAGCGTGGTGTACTTCGCCATCATCACCTGCTGTCTGCTGC
GGCGGACCGCCTTCTGCTGCAACGGCGAGAAGAGCGGCTCTGGAGCTACTAACTTCAGCCTGCTGAAG
CAGGCTGGAGACGTGGAGGAGAACCCTGGACCTATGCAGAGGATTTCCAGTCTCATTCACCTCAGCTT
GTTTTGGGCAGGCGTCATGTCTACTGACTTCGAGGTGAAAACCGACTCCACCGACCACGTGAAGCCCA
AAGAGACCGAGAACACCAAGCAGCCCAGCAAGAGCTGCCACAAGCCCAAGGCCATCGTGCACACCGAG
AAGGTGAACATGATGAGCCTGACCGTGCTGGGCCTGCGGATGCTGTTCGCCAAGACAGTGGCCGTGAA
CTTCCTGCTGACCGCCAAGCTGTTCTTCCTG
Protein
Artificial
Precursor PD-L1 scFv-γδTCR polypeptides encoded by
SEQ ID NO: 21
MEWSWVFLFFLSVTTGVHSEVQLVESGAEVKKPGSSVKVSCKASGGTFSSYAISWVRQAPGQ
GLEWMGGIIPIFGTANYAQKFQGRVTITADESTSTAYMELSSLRSEDTAVYYCARAPYYYYY
MDVWGQGTTVTVSSGGGGSGGGGSGGGGSQSALTQPASVSGSLGQSVTISCTGSSSDVGSYN
LVSWYQQHPGKAPNLMIYDVSKRSGVSNRFSGSKSGNTASLTISGLQAEDEADYYCSSYTGI
STVVFGGGTKLIVLPIKTDVITMDPKDNCSKDANDILLLQLTNTSAYYMYLLLLLKSVVYFA
IITCCLLRRTAFCCNGEKSGSGEGRGSLLTCGDVEENPGPMQRISSLIHLSLFWAGVMSTDF
EVKTDSTDHVKPKETENTKQPSKSCHKPKAIVHTEKVNMMSLTVLGLRMLFAKTVAVNFLLT
AKLFFL
Protein
Mus musculus
Signal peptide (Heavy chain IgG leader sequence)
SEQ ID NO: 22
MEWSWVFLFFLSVTTGVHS
Protein
Artificial
Signal peptide
SEQ ID NO: 23
MQRISSLIHLSLFWAGVMSTDE
Protein
Artificial
Precursor Chimeric anti-PD-L1 scFv-TCRγ subunit
SEQ ID NO: 24
MEWSWVFLFFLSVTTGVHSEVQLVESGAEVKKPGSSVKVSCKASGGTFSSYAISWVRQAPGQ
GLEWMGGIIPIFGTANYAQKFQGRVTITADESTSTAYMELSSLRSEDTAVYYCARAPYYYYY
MDVWGQGTTVTVSSGGGGSGGGGSGGGGSQSALTQPASVSGSLGQSVTISCTGSSSDVGSYN
LVSWYQQHPGKAPNLMIYDVSKRSGVSNRFSGSKSGNTASLTISGLQAEDEADYYCSSYTGI
STVVFGGGTKLTVLPIKTDVITMDPKDNCSKDANDTLLLQLTNTSAYYMYLLLLLKSVVYFA
IITCCLLRRTAFCCNGEKS
Protein
Artificial
Mature Chimeric anti-PD-L1 scFv-TCRγ subunit
SEQ ID NO: 25
EVQLVESGAEVKKPGSSVKVSCKASGGTFSSYAISWVRQAPGQGLEWMGGIIPIFGTANYAQKFQGRV
TITADESTSTAYMELSSLRSEDTAVYYCARAPYYYYYMDVWGQGTTVTVSSGGGGSGGGGSGGGGSQS
ALTQPASVSGSLGQSVTISCTGSSSDVGSYNLVSWYQQHPGKAPNLMIYDVSKRSGVSNRFSGSKSGN
TASLTISGLQAEDEADYYCSSYTGISTVVFGGGTKLTVLPIKTDVITMDPKDNCSKDANDTLLLQLTN
TSAYYMYLLLLLKSVVYFAIITCCLLRRTAFCCNGEKS
Protein
Artificial
Precursor N-terminally truncated TCRδ subunit
SEQ ID NO: 26
MQRISSLIHLSLFWAGVMSTDFEVKTDSTDHVKPKETENTKQPSKSCHKPKAIVHTEKVNMMSLTVLG
LRMLFAKTVAVNFLLTAKLFFL
Protein
Artificial
Mature N-terminally truncated TCRδ subunit
SEQ ID NO: 27
EVKTDSTDHVKPKETENTKQPSKSCHKPKAIVHTEKVNMMSLTVLGLRMLFAKTVAVNFLLTAKLFFL
DNA
Artificial
PD-L1 CAR construct
SEQ ID NO: 28
ATGGAGTGGTCCTGGGTGTTCCTGTTCTTTCTGTCCGTGACCACCGGTGTCCACTCTGAAGTCCAACT
GGTGGAATCTGGAGCCGAGGTAAAGAAACCTGGCTCAAGTGTAAAAGTATCATGCAAAGCGTCCGGCG
GCACGTTTTCAAGTTACGCCATAAGTTGGGTGAGACAAGCTCCCGGACAGGGACTTGAGTGGATGGGA
GGAATTATACCTATTTTTGGGACTGCGAATTATGCACAAAAGTTCCAGGGAAGGGTTACTATCACAGC
GGACGAGAGTACAAGTACAGCCTATATGGAGTTGTCTAGTCTCAGATCTGAGGACACTGCTGTCTATT
ATTGTGCGCGAGCGCCTTACTATTACTACTATATGGACGTATGGGGCCAAGGCACTACGGTCACAGTA
AGTTCTGGCGGCGGCGGCAGCGGTGGCGGTGGCTCAGGTGGTGGTGGTTCTCAGAGTGCCCTTACTCA
GCCTGCCAGTGTATCCGGTTCACTTGGACAATCAGTTACAATATCTTGTACGGGCTCTTCATCCGACG
TGGGCAGTTATAATCTCGTTTCTTGGTATCAACAACACCCCGGCAAAGCACCCAATCTGATGATCTAC
GATGTCAGTAAACGAAGCGGTGTCAGTAACAGGTTCTCAGGTTCAAAATCCGGCAATACCGCGAGTCT
TACAATTTCCGGTTTGCAGGCGGAGGATGAGGCCGACTACTATTGTTCATCCTATACAGGTATTAGCA
CCGTTGTGTTTGGCGGGGGCACTAAACTCACTGTTCTCGCTAAGCCGACCACGACACCGGCTCCAAGA
CCTCCGACGCCAGCTCCAACGATAGCGTCACAGCCATTGTCTCTCCGCCCTGAAGCCTGCCGGCCCGC
TGCGGGCGGCGCGGTTCATACCCGGGGATTGGACTTTGCCCCCAGAAAGATAGAGGTGATGTACCCTC
CCCCCTACTTGGACAACGAAAAGTCTAATGGCACTATCATTCACGTAAAGGGCAAACACCTTTGTCCA
AGTCCTTTGTTCCCAGGCCCATCTAAGCCGTTCTGGGTACTCGTGGTTGTGGGGGGCGTGCTCGCTTG
TTACTCACTGCTGGTGACGGTGGCCTTTATTATTTTCTGGGTTAAACGGGGCAGAAAGAAACTCCTGT
ATATATTCAAACAACCATTTATGAGACCAGTACAAACTACTCAAGAGGAAGATGGCTGTAGCTGCCGA
TTTCCAGAAGAAGAAGAAGGAGGATGTGAACTGAGGGTAAAATTTAGCAGGTCTGCAGATGCGCCTGC
GTATCAACAGGGTCAGAATCAGCTCTATAATGAGCTGAACCTCGGGCGGCGGGAAGAGTATGATGTTC
TCGATAAAAGGAGAGGACGAGACCCCGAAATGGGCGGCAAACCGAGACGCAAAAATCCTCAGGAGGGG
CTCTACAATGAACTTCAAAAAGACAAAATGGCCGAAGCATACTCAGAAATCGGAATGAAAGGGGAGAG
GAGACGCGGGAAGGGCCATGATGGACTGTATCAGGGACTTTCCACAGCCACCAAGGACACCTATGACG
CTCTCCACATGCAGGCCCTGCCCCCTCGC
Protein
Artificial
PD-L1 CAR (Precursor)
SEQ ID NO: 29
MEWSWVFLFFLSVTTGVHSEVQLVESGAEVKKPGSSVKVSCKASGGTFSSYAISWVRQAPGQGLEWMG
GIIPIFGTANYAQKFQGRVTITADESTSTAYMELSSLRSEDTAVYYCARAPYYYYYMDVWGQGTTVTV
SSGGGGSGGGGSGGGGSQSALTQPASVSGSLGQSVTISCTGSSSDVGSYNLVSWYQQHPGKAPNLMIY
DVSKRSGVSNRFSGSKSGNTASLTISGLQAEDEADYYCSSYTGISTVVFGGGTKLTVLAKPTTTPAPR
PPTPAPTIASQPLSLRPEACRPAAGGAVHTRGLDFAPRKIEVMYPPPYLDNEKSNGTIIHVKGKHLCP
SPLFPGPSKPFWVLVVVGGVLACYSLLVTVAFIIFWVKRGRKKLLYIFKQPFMRPVQTTQEEDGCSCR
FPEEEEGGCELRVKFSRSADAPAYQQGQNQLYNELNLGRREEYDVLDKRRGRDPEMGGKPRRKNPQEG
LYNELQKDKMAEAYSEIGMKGERRRGKGHDGLYQGLSTATKDTYDALHMQALPPR
Protein
Homo sapiens
CD8a hinge region
SEQ ID NO: 30
AKPTTTPAPRPPTPAPTIASQPLSLRPEACRPAAGGAVHTRGLDFA
Protein
Homo sapiens
CD28 hinge region
SEQ ID NO: 31
PRKIEVMYPPPYLDNEKSNGTIIHVKGKHLCPSPLFPGPSKP
Protein
Homo sapiens
CD28 transmembrane domain
SEQ ID NO: 32
FWVLVVVGGVLACYSLLVTVAFIIFWV
Protein
Homo sapiens
CD3zeta (includes ITAMs 1, 2, & 3)
SEQ ID NO: 33
RVKFSRSADAPAYQQGQNQLYNELNLGRREEYDVLDKRRGRDPEMGGKPRRKNPQEGLYNEL
QKDKMAEAYSEIGMKGERRRGKGHDGLYQGLSTATKDTYDALHMQALPPR
Protein
Homo sapiens
4-1BB co-stimulatory sequence
SEQ ID NO: 34
KRGRKKLLYIFKQPFMRPVQTTQEEDGCSCRFPEEEEGGCEL
DNA
Artificial
JeT promoter
SEQ ID NO: 35
GAATTCGGGCGGAGTTAGGGCGGAGCCAATCAGCGTGCGCCGTTCCGAAAGTTGCCTTTTAT
GGCTGGGCGGAGAATGGGCGGTGAACGCCGATGATTATATAAGGACGCGCCGGGTGTGGCAC
AGCTAGTTCCGTCGCAGCCGGGATTTGGGTCGCGGTTCTTGTTTGTGGATCCCTGTGATCGT
CAGTTGACA
DNA
Cytomegalovirus
CMV promoter
SEQ ID NO: 36
AAGCTTGGGAGTTCCGCGTTACATAACTTACGGTAAATGGCCCGCCTGGCTGACCGCCCAAC
GACCCCCGCCCATTGACGTCAATAATGACGTATGTTCCCATAGTAACGCCAATAGGGACTTT
CCATTGACGTCAATGGGTGGAGTATTTACGGTAAACTGCCCACTTGGCAGTACATCAAGTGT
ATCATATGCCAAGTACGCCCCCTATTGACGTCAATGACGGTAAATGGCCCGCCTGGCATTAT
GCCCAGTACATGACCTTATGGGACTTTCCTACTTGGCAGTACATCTACGTATTAGTCATCGC
TATTACCATGGTGATGCGGTTTTGGCAGTACATCAATGGGCGTGGATAGCGGTTTGACTCAC
GGGGATTTCCAAGTCTCCACCCCATTGACGTCAATGGGAGTTTGTTTTGGCACCAAAATCAA
CGGGACTTTCCAAAATGTCGTAACAACTCCGCCCCATTGACGCAAATGGGCGGTAGGCGTGT
ACGGTGGGAGGTCTATATAAGCAGAGCTCGTTTAGTGAACCGTCAGATCGCCTGGAGACGCC
ATCCACGCTGTTTTGACCTCCATAGAAGACACCGACTCTACTAGA
DNA
Homo sapiens
5′ homology arm from exon 1 of TRAC gene, Cas9 target site, 660 nt
SEQ ID NO: 37
GGCACCATATTCATTTTGCAGGTGAAATTCCTGAGATGTAAGGAGCTGCTGTGACTTGCTCA
AGGCCTTATATCGAGTAAACGGTAGTGCTGGGGCTTAGACGCAGGTGTTCTGATTTATAGTT
CAAAACCTCTATCAATGAGAGAGCAATCTCCTGGTAATGTGATAGATTTCCCAACTTAATGC
CAACATACCATAAACCTCCCATTCTGCTAATGCCCAGCCTAAGTTGGGGAGACCACTCCAGA
TTCCAAGATGTACAGTTTGCTTTGCTGGGCCTTTTTCCCATGCCTGCCTTTACTCTGCCAGA
GTTATATTGCTGGGGTTTTGAAGAAGATCCTATTAAATAAAAGAATAAGCAGTATTATTAAG
TAGCCCTGCATTTCAGGTTTCCTTGAGTGGCAGGCCAGGCCTGGCCGTGAACGTTCACTGAA
ATCATGGCCTCTTGGCCAAGATTGATAGCTTGTGCCTGTCCCTGAGTCCCAGTCCATCACGA
GCAGCTGGTTTCTAAGATGCTATTTCCCGTATAAAGCATGAGACCGTGACTTGCCAGCCCCA
CAGAGCCCCGCCCTTGTCCATCACTGGCATCTGGACTCCAGCCTGGGTTGGGGCAAAGAGGG
AAATGAGATCATGTCCTAACCCTGATCCTCTTGTCCCACA
DNA
Homo sapiens
3′ homology arm from exon 1 of TRAC gene, Cas9 target site, 650 nt
SEQ ID NO: 38
GATATCCAGAACCCTGACCCTGCCGTGTACCAGCTGAGAGACTCTAAATCCAGTGACAAGTC
TGTCTGCCTATTCACCGATTTTGATTCTCAAACAAATGTGTCACAAAGTAAGGATTCTGATG
TGTATATCACAGACAAAACTGTGCTAGACATGAGGTCTATGGACTTCAAGAGCAACAGTGCT
GTGGCCTGGAGCAACAAATCTGACTTTGCATGTGCAAACGCCTTCAACAACAGCATTATTCC
AGAGGACACCTTCTTCCCCAGCCCAGGTAAGGGCAGCTTTGGTGCCTTCGCAGGCTGTTTCC
TTGCTTCAGGAATGGCCAGGTTCTGCCCAGAGCTCTGGTCAATGATGTCTAAAACTCCTCTG
ATTGGTGGTCTCGGCCTTATCCATTGCCACCAAAACCCTCTTTTTACTAAGAAACAGTGAGC
CTTGTTCTGGCAGTCCAGAGAATGACACGGGAAAAAAGCAGATGAAGAGAAGGTGGCAGGAG
AGGGCACGTGGCCCAGCCTCAGTCTCTCCAACTGAGTTCCTGCCTGCCTGCCTTTGCTCAGA
CTGTTTGCCCCTTACTGCTCTTCTAGGCCTCATTCTAAGCCCCTTCTCCAAGTTGCCTCTCC
TTATTTCTCCCTGTCTGCCAAAAAATCTTT
DNA
Homo sapiens
Cas9 target site , TRAC locus
SEQ ID NO: 39
CAGGGTTCTGGATATCTGT
DNA
Artificial
Forward primer
SEQ ID NO: 40
A*TmC*mA*mCGAGCAGCTGGTTTCT
(*indicates phosphorothioate linkage; mA indicates
2′-O-methyladenosine; mC indicates 2′-O-methylcytidine)
DNA
Artificial
Reverse primer
SEQ ID NO: 41
GACCTCATGTCTAGCACAGTTTTG
DNA
Homo sapiens
171 bp 5′ homology region, Cas9 target site
SEQ ID NO: 42
ATCACGAGCAGCTGGITTCTAAGATGCTATTTCCCGTATAAAGCATGAGACCGTGACTTGCC
AGCCCCACAGAGCCCCGCCCTTGTCCATCACTGGCATCTGGACTCCAGCCTGGGTTGGGGCA
AAGAGGGAAATGAGATCATGTCCTAACCCTGATCCTCTTGTCCCACA
DNA
Homo sapiens
161 bp 3′ homology region, Cas9 target site
SEQ ID NO: 43
GATATCCAGAACCCTGACCCTGCCGTGTACCAGCTGAGAGACTCTAAATCCAGTGACAAGTCTGTCTG
CCTATTCACCGATTTTGATTCTCAAACAAATGTGTCACAAAGTAAGGATTCTGATGTGTATATCACAG
ACAAAACTGTGCTAGACATGAGGTC
Protein
Artificial
Alternative GS linker
SEQ ID NO: 44
GGGSGGGSGGGSGGGSG
Protein
Artificial
Alternative linker
SEQ ID NO: 45
(GGGSE)n
n ranges from 1 to 20
Protein
Artificial
Anti-PD-L1 Heavy Chain CDR1
SEQ ID NO: 46
SYAIS
Protein
Artificial
SEQ ID NO: 47
GIIPIFGTANYAQKFQG
Protein
Artificial
Anti-PD-L1 Heavy Chain CDR3
SEQ ID NO: 48
APYYYYYMDV
Protein
Artificial
Anti-PD-L1 Light Chain CDR1
SEQ ID NO: 49
TGSSSDVGSYNLVS
Protein
Artificial
Anti-PD-L1 Light Chain CDR2
SEQ ID NO: 50
DVSKRS
Protein
Artificial
Anti-PD-L1 Light Chain CDR3
SEQ ID NO: 51
SSYTGISTVV
Protein
Artificial
P2A self-cleaving sequence
SEQ ID NO: 52
GSGATNFSLLKQAGDVEENPGP
Protein
Artificial
E2A self-cleaving sequence
SEQ ID NO: 53
GSGQCTNYALLKLAGDVESNPGP
Protein
Artificial
F2A self-cleaving sequence
SEQ ID NO: 54
GSGVKQTLNFDLLKLAGDVESNPGP
Protein
Homo sapiens
CD8a leader sequence
SEQ ID NO: 55
MALPVTALLLPLALLLHAARP
Protein
Artificial
Signal Peptide
SEQ ID NO: 56
MEFGLSWVFLVALFRGVQCD
Protein
Artificial
Signal Peptide
SEQ ID NO: 57
METDTLLLWVLLLWVP
Protein
Artificial
Heavy chain variable region anti-CD19 FMC63 antibody
SEQ ID NO: 58
EVKLQESGPGLVAPSQSLSVTCTVSGVSLPDYGVSWIRQPPRKGLEWLGVIWGSETTYYNSA
LKSRLTIIKDNSKSQVFLKMNSLQTDDTAIYYCAKHYYYGGSYAMDYWGQGTSVTVSS
Protein
Artificial
Light chain variable region anti-CD19 FMC63 antibody
SEQ ID NO: 59
DIQMTQTTSSLSASLGDRVTISCRASQDISKYLNWYQQKPDGTVKLLIYHTSRLHSGVPSRF
SGSGSGTDYSLTISNLEQEDIATYFCQQGNTLPYTFGGGTKLEIT
Protein
Artificial
anti-CD19 ScFv
SEQ ID NO: 60
EVKLQESGPGLVAPSQSLSVTCTVSGVSLPDYGVSWIRQPPRKGLEWLGVIWGSETTYYNSA
LKSRLTIIKDNSKSQVFLKMNSLQTDDTAIYYCAKHYYYGGSYAMDYWGQGTSVTVSSGGGG
SGGGGSGGGGSDIQMTQTTSSLSASLGDRVTISCRASQDISKYLNWYQQKPDGTVKLLIYHT
SRLHSGVPSRFSGSGSGTDYSLTISNLEQEDIATYFCQQGNTLPYTFGGGTKLEIT
Protein
Homo sapiens
CD8a transmembrane domain
SEQ ID NO: 61
IYIWAPLAGTCGVLLLSLVITLY
Protein
Artificial
Mature Chimeric anti-CD19 scFv-TCRγ subunit
SEQ ID NO: 62
EVKLQESGPGLVAPSQSLSVTCTVSGVSLPDYGVSWIRQPPRKGLEWLGVIWGSETTYYNSA
LKSRLIIIKDNSKSQVFLKMNSLQTDDTAIYYCAKHYYYGGSYAMDYWGQGTSVTVSSGGGG
SGGGGSGGGGSDIQMTQTTSSLSASLGDRVTISCRASQDISKYLNWYQQKPDGTVKLLIYHT
SRLHSGVPSRFSGSGSGTDYSLTISNLEQEDIATYFCQQGNTLPYTFGGGTKLEITPIKTDV
ITMDPKDNCSKDANDTLLLQLTNTSAYYMYLLLLLKSVVYFAIITCCLLRRTAFCCNGEKS
Protein
Artificial
Precursor Chimeric anti-CD19 scFv-TCRγ subunit
SEQ ID NO: 63
MEWSWVFLFFLSVTTGVHSEVKLQESGPGLVAPSQSLSVTCTVSGVSLPDYGVSWIRQPPRK
GLEWLGVIWGSETTYYNSALKSRLTIIKDNSKSQVFLKMNSLQTDDTAIYYCAKHYYYGGSY
AMDYWGQGISVTVSSGGGGSGGGGSGGGGSDIQMTQTTSSLSASLGDRVTISCRASQDISKY
LNWYQQKPDGTVKLLIYHTSRLHSGVPSRFSGSGSGTDYSLTISNLEQEDIATYFCQQGNTL
PYTFGGGTKLEITPIKTDVITMDPKDNCSKDANDTLLLQLINTSAYYMYLLLLLKSVVYFAI
ITCCLLRRTAFCCNGEKS
DNA
Artificial
Encodes precursor Chimeric anti-CD19 scFv-TCRγ subunit
SEQ ID NO: 64
ATGTCCGTCCCTACCCAGGTGCTGGGCCTGCTGCTGCTGTGGCTGACCGATGCTA
GATGCGATATCCAGATGACACAGACCACCAGCAGCCTGAGCGCCAGCCTGGGCG
ACCGAGTGACTATCAGCTGCCGGGCATCCCAGGATATTTCTAAGTATCTGAACTG
GTACCAGCAGAAGCCCGACGGCACTGTCAAACTGCTGATCTACCACACCAGTAG
ACTGCATTCAGGGGTGCCTAGCAGGTTCTCCGGATCTGGCAGTGGGACTGACTAC
TCCCTGACCATCTCTAACCTGGAGCAGGAAGATATTGCCACCTATTTCTGCCAGC
AGGGCAATACACTGCCTTACACTTTTGGCGGGGGAACAAAGCTGGAGATCACTgg
cggcggcggcagcggtggcggtggctcaggtggtggtggttctGAGGTGAAACTGCA
GGAAAGCGGACCAGGACTGGTCGCACCTTCACAGAGCCTGTCCGTGACATGTACTGT
CTCCGGAGTGTCTCTGCCCGATTACGGCGTCTCTTGGATCCGGCAGCCCCCTAGAAA
GGGACTGGAGTGGCTGGGCGTGATCTGGGGAAGTGAAACTACCTACTATAATAGTGC
TCTGAAATCAAGACTGACCATCATTAAGGACAACTCTAAAAGTCAGGTGTTTCTGAA
GATGAATTCCCTGCAGACCGACGATACAGCAATCTACTATTGCGCCAAACACTACT
ATTACGGCGGGAGCTATGCCATGGATTACTGGGGGCAGGGAACTTCCGTCACCGT
GAGCAGCcccatcaagaccgatgtgatcacaatggaccccaaggacaactgcagcaa
ggacgccaacgataccctgctgctgcagctgaccaacaccagcgcctactacatgta
tctcctgctgctgctgaagagcgtggtgtacttcgccatcatcacctgctgtctgct
gcggcggaccgccttctgctgcaacggcgagaagagc
DNA
Artificial
Encodes mature Chimeric anti-CD19 scFv-TCRγ subunit
(without signal peptide)
SEQ ID NO: 65
GATATCCAGATGACACAGACCACCAGCAGCCTGAGCGCCAGCCTGGGCGACCGA
GTGACTATCAGCTGCCGGGCATCCCAGGATATTTCTAAGTATCTGAACTGGTACC
AGCAGAAGCCCGACGGCACTGTCAAACTGCTGATCTACCACACCAGTAGACTGC
ATTCAGGGGTGCCTAGCAGGTTCTCCGGATCTGGCAGTGGGACTGACTACTCCCT
GACCATCTCTAACCTGGAGCAGGAAGATATTGCCACCTATTTCTGCCAGCAGGGC
AATACACTGCCTTACACTTTTGGCGGGGGAACAAAGCTGGAGATCACTggcggcggc
ggcagcggtggcggtggctcaggtggtggtggttctGAGGTGAAACTGCAGGAAAGC
GGACCAGGACTGGTCGCACCTTCACAGAGCCTGTCCGTGACATGTACTGTCTCCGGA
GTGTCTCTGCCCGATTACGGCGTCTCTTGGATCCGGCAGCCCCCTAGAAAGGGACTG
GAGTGGCTGGGCGTGATCTGGGGAAGTGAAACTACCTACTATAATAGTGCTCTGAAA
TCAAGACTGACCATCATTAAGGACAACTCTAAAAGTCAGGTGTTTCTGAAGATGAA
TTCCCTGCAGACCGACGATACAGCAATCTACTATTGCGCCAAACACTACTATTAC
GGCGGGAGCTATGCCATGGATTACTGGGGGCAGGGAACTTCCGTCACCGTGAGC
AGCcccatcaagaccgatgtgatcacaatggaccccaaggacaactgcagcaaggac
gccaacgataccctgctgctgcagctgaccaacaccagcgcctactacatgtatctc
ctgctgctgctgaagagcgtggtgtacttcgccatcatcacctgctgtctgctgcgg
cggaccgccttctgctgcaacggcgagaagagc

1. A host cell, or population of host cells, comprising an exogenous nucleic acid sequence encoding a chimeric single chain antibody TCR gamma subunit (scFv-TCRγ), wherein the scFv-TCRγ subunit comprises, from the N-terminus to the C-terminus:
a) an scFv that binds a target antigen; and
b) a combined TCRγ connecting peptide, TCRγ transmembrane domain, and TCRγ intracellular domain sequence having at least 95% identity to SEQ ID NO:17;
or
a TCRγ connecting peptide having at least 95% identity to SEQ ID NO:9,
a TCRγ transmembrane domain having at least 95% identity to SEQ ID NO:10, and a TCRγ intracellular domain having at least 95% identity to SEQ ID NO:11;
wherein the host cell or population of host cells does not comprise an exogenous nucleic acid sequence encoding a polypeptide that comprises the transmembrane domain of a TCR delta (TCRδ) subunit or an amino acid sequence having at least 95% identity thereto or a polypeptide that comprises the connecting peptide of a TCRδ subunit or an amino acid sequence having at least 95% identity thereto.

2. The host cell or population of host cells of claim 1, wherein the host cell or population of host cells does not comprise an exogenous nucleic acid sequence encoding a polypeptide that comprises the transmembrane domain of a TCRδ subunit or an amino acid sequence having at least 95% identity to the transmembrane domain of a TCRδ subunit.

3. The host cell or population of host cells of claim 1, wherein the host cell or population of host cells does not comprise an exogenous nucleic acid sequence encoding a polypeptide that comprises the connecting peptide of a TCRδ subunit or an amino acid sequence having at least 95% identity to the connecting peptide of a TCRδ subunit.

4. The host cell or population of host cells of claim 1, wherein the host cell(s) do not express an engineered or non-engineered TCRδ subunit.

5. The host cell or population of host cells of claim 1, wherein the scFv-TCRγ subunit comprises, from the N-terminus to the C-terminus: an scFv that binds a target antigen; and a combined TCRγ connecting peptide, TCRγ transmembrane domain, and TCRγ intracellular domain sequence comprising an amino acid sequence having at least 95% identity to SEQ ID NO:17.

6. The host cell or population of host cells of claim 1, wherein the scFv-TCRγ subunit comprises, from the N-terminus to the C-terminus: an scFv that binds a target antigen; and a TCRγ connecting peptide having at least 95% identity to SEQ ID NO:9, a TCRγ transmembrane domain having at least 95% identity to SEQ ID NO:10, and a TCRγ intracellular domain having at least 95% identity to SEQ ID NO:11.

7. The host cell or population of host cells of claim 6, wherein the scFv-TCRγ subunit comprises, from the N-terminus to the C-terminus:
an scFv that binds a target antigen; and
a TCRγ connecting peptide having at least 95% identity to SEQ ID NO:9,
a TCRγ transmembrane domain having at least 95% identity to SEQ ID NO:10, and
a TCRγ intracellular domain comprising the sequence of SEQ ID NO:11.

8. The host cell or population of host cells of claim 6, wherein the scFv-TCRγ subunit comprises, from the N-terminus to the C-terminus:
an scFv that binds a target antigen; and
a TCRγ connecting peptide having at least 95% identity to SEQ ID NO:9,
a TCRγ transmembrane domain comprising the sequence of SEQ ID NO:10, and
a TCRγ intracellular domain having at least 95% identity to SEQ ID NO:11.

9. The host cell or population of host cells of claim 6, wherein the scFv-TCRγ subunit comprises, from the N-terminus to the C-terminus:
an scFv that binds a target antigen; and
a TCRγ connecting peptide comprising the sequence of SEQ ID NO:9;
a TCRγ transmembrane domain having at least 95% identity to SEQ ID NO:10; and
a TCRγ intracellular domain having at least 95% identity to SEQ ID NO:11.

10. (canceled)

11. The host cell or population of host cells of claim 1, wherein the target antigen is a tumor associated antigen or an immune checkpoint protein.

12. (canceled)

13. The host cell or population of host cells of claim 1, wherein the target antigen is PD-L1, B7H3, BCMA, CD19, CD20, CD22, CD38, CD123, Claudin 18.2, EGFRVIII, GPC3, mesothelin, MUC1, or PSMA.

14. The host cell or population of host cells of claim 13, wherein the target antigen is PD-L1.

15. A host cell, or population of host cells, comprising:
(a) an exogenous nucleic acid sequence encoding a chimeric single chain antibody TCR gamma subunit (scFv-TCRγ), wherein the scFv-TCRγ subunit comprises a combined scFv, TCRγ connecting peptide, TCRγ transmembrane domain, and TCRγ intracellular domain sequence having at least 95% identity to SEQ ID NO:25, wherein the host cell or population of host cells does not comprise an exogenous nucleic acid sequence encoding a polypeptide that comprises a transmembrane domain of a TCRδ subunit or a sequence having at least 95% identity thereto or a connecting peptide region of a TCRδ subunit or a sequence having at least 95% identity thereto; or
(b) an exogenous nucleic acid sequence encoding a chimeric single chain antibody TCR gamma subunit (scFv-TCRγ), wherein the scFv-TCRγ subunit comprises a combined scFv, TCRγ connecting peptide, TCRγ transmembrane domain, and TCRγ intracellular domain sequence having at least 95% identity to SEQ ID NO:62.

16. The host cell or population of host cells claim 15, wherein the scFv comprises a heavy chain variable region comprising a heavy chain complementarity determining region (HCDR1) comprising SEQ ID NO:46, an HCDR2 comprising SEQ ID NO:47, and an HCDR3 comprising SEQ ID NO:48, and a light chain variable region comprising a light chain complementarity determining region (LCDR1) comprising SEQ ID NO:49, an LCDR2 comprising SEQ ID NO:50, and an LCDR3 comprising SEQ ID NO:51.

17. The host cell or population of host cells of claim 15, wherein the scFv comprises a heavy chain variable region having at least 95% identity to SEQ ID NO:1 and a light chain variable region having at least 95% identity to SEQ ID NO:2, optionally wherein the heavy chain variable region comprises the sequence of SEQ ID NO:1 and the light chain variable region comprises the sequence of SEQ ID NO:2.

18. The host cell or population of host cells of claim 15, wherein the scFv comprises an amino acid sequence having at least 95% identity to SEQ ID NO:4, optionally wherein the scFv comprises the sequence of SEQ ID NO:4.

19. The host cell or population of host cells of claim 1, wherein the target antigen is CD19.

20-28. (canceled)

29. A pharmaceutical composition comprising a host cell or a population of host cells according to claim 1.

30-37. (canceled)

38. A method of treating a subject with cancer, comprising administering a pharmaceutical composition according to claim 29 to a subject with cancer.

39-44. (canceled)

45. A recombinant nucleic acid molecule encoding a chimeric scFv-TCRγ, wherein:
(a) the scFv-TCRγ subunit comprises, from the N-terminus to the C-terminus:
an scFv that binds a target antigen; and
a combined connecting peptide, transmembrane domain, and intracellular domain sequence having at least 95% identity to SEQ ID NO:17; or
a TCRγ connecting peptide having at least 95% identity to SEQ ID NO:9,
a TCRγ transmembrane domain having at least 95% identity to SEQ ID NO:10, and a TCRγ intracellular domain having at least 95% identity to SEQ ID NO:11;
(b) the chimeric scFv-TCRγ comprises a combined scFv, TCRγ connecting peptide, TCRγ transmembrane domain, and TCRγ intracellular domain sequence having at least 95% identity to SEQ ID NO:25; or
(c) the chimeric scFv-TCRγ comprises a combined scFv, TCRγ connecting peptide, TCRγ transmembrane domain, and TCRγ intracellular domain sequence having at least 95% identity to SEQ ID NO:63.

46-63. (canceled)


SEQUENCE LISTING (65 sequences)

SEQ ID NO: 1 - protein, 119 aa, synthetic construct
  REGION 1..119: note = SH1E2 PD-L1 antibody heavy chain variable region
EVQLVESGAEVKKPGSSVKVSCKASGGTFSSYAISWVRQAPGQGLEWMGGIIPIFGTANYAQKFQGRVTITADESTSTAYMELSSLRSEDTAVYYCARAPYYYYYMDVWGQGTTVTVSS

SEQ ID NO: 2 - protein, 109 aa, synthetic construct
  REGION 1..109: note = SH1E2 PD-L1 antibody light chain variable region
QSALTQPASVSGSLGQSVTISCTGSSSDVGSYNLVSWYQQHPGKAPNLMIYDVSKRSGVSNRFSGSKSGNTASLTISGLQAEDEADYYCSSYTGISTVVFGGGTKLTVL

SEQ ID NO: 3 - protein, 15 aa, synthetic construct
  REGION 1..15: note = (G4S)3 linker
GGGGSGGGGSGGGGS

SEQ ID NO: 4 - protein, 243 aa, synthetic construct
  REGION 1..243: note = PD-L1 scFv; SH1E2 heavy chain-(G4S)3 -SH1E2 light chain
EVQLVESGAEVKKPGSSVKVSCKASGGTFSSYAISWVRQAPGQGLEWMGGIIPIFGTANYAQKFQGRVTITADESTSTAYMELSSLRSEDTAVYYCARAPYYYYYMDVWGQGTTVTVSSGGGGSGGGGSGGGGSQSALTQPASVSGSLGQSVTISCTGSSSDVGSYNLVSWYQQHPGKAPNLMIYDVSKRSGVSNRFSGSKSGNTASLTISGLQAEDEADYYCSSYTGISTVVFGGGTKLTVL

SEQ ID NO: 5 - protein, 243 aa, synthetic construct
  REGION 1..243: note = PD-L1 scFv; SH1E2 light chain-(G4S)3 -SH1E2 heavy chain
QSALTQPASVSGSLGQSVTISCTGSSSDVGSYNLVSWYQQHPGKAPNLMIYDVSKRSGVSNRFSGSKSGNTASLTISGLQAEDEADYYCSSYTGISTVVFGGGTKLTVLGGGGSGGGGSGGGGSEVQLVESGAEVKKPGSSVKVSCKASGGTFSSYAISWVRQAPGQGLEWMGGIIPIFGTANYAQKFQGRVTITADESTSTAYMELSSLRSEDTAVYYCARAPYYYYYMDVWGQGTTVTVSS

SEQ ID NO: 6 - DNA, 1326 bp, synthetic construct
  misc_feature 1..1326: note = Construct that includes sequence encoding Signal peptide-Truncated gamma9 polypeptide-T2A sequence-Chimeric anti-PD-L1 ScFv/delta2 polypeptide
  source 1..1326: mol_type = other DNA
atgttgagtttgctccacacgtcaacattggcagtcctgggggctctgtgcgtgtacggattcccgcccatcaagaccgatgtgatcacaatggaccccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagcggctctggagctactaacttcagcctgctgaagcaggctggagacgtggaggagaaccctggacctatgtccgtccctacccaggtgctgggcctgctgctgctgtggctgaccgatgctagatgcgaagtccaactggtggaatctggagccgaggtaaagaaacctggctcaagtgtaaaagtatcatgcaaagcgtccggcggcacgttttcaagttacgccataagttgggtgagacaagctcccggacagggacttgagtggatgggaggaattatacctatttttgggactgcgaattatgcacaaaagttccagggaagggttactatcacagcggacgagagtacaagtacagcctatatggagttgtctagtctcagatctgaggacactgctgtctattattgtgcgcgagcgccttactattactactatatggacgtatggggccaaggcactacggtcacagtaagttctggcggcggcggcagcggtggcggtggctcaggtggtggtggttctcagagtgcccttactcagcctgccagtgtatccggttcacttggacaatcagttacaatatcttgtacgggctcttcatccgacgtgggcagttataatctcgtttcttggtatcaacaacaccccggcaaagcacccaatctgatgatctacgatgtcagtaaacgaagcggtgtcagtaacaggttctcaggttcaaaatccggcaataccgcgagtcttacaatttccggtttgcaggcggaggatgaggccgactactattgttcatcctatacaggtattagcaccgttgtgtttggcgggggcactaaactcactgttctcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctg

SEQ ID NO: 7 - protein, 441 aa, synthetic construct
  REGION 1..441: note = Precursor PD-L1 scFv-gamma-delta-TCR polypeptide encoded by SEQ ID NO:6
MLSLLHTSTLAVLGALCVYGFPPIKTDVITMDPKDNCSKDANDTLLLQLTNTSAYYMYLLLLLKSVVYFAIITCCLLRRTAFCCNGEKSGSGEGRGSLLTCGDVEENPGPMSVPTQVLGLLLLWLTDARCEVQLVESGAEVKKPGSSVKVSCKASGGTFSSYAISWVRQAPGQGLEWMGGIIPIFGTANYAQKFQGRVTITADESTSTAYMELSSLRSEDTAVYYCARAPYYYYYMDVWGQGTTVTVSSGGGGSGGGGSGGGGSQSALTQPASVSGSLGQSVTISCTGSSSDVGSYNLVSWYQQHPGKAPNLMIYDVSKRSGVSNRFSGSKSGNTASLTISGLQAEDEADYYCSSYTGISTVVFGGGTKLTVLEVKTDSTDHVKPKETENTKQPSKSCHKPKAIVHTEKVNMMSLTVLGLRMLFAKTVAVNFLLTAKLFFL

SEQ ID NO: 8 - protein, 22 aa, synthetic construct
  REGION 1..22: note = Signal Peptide
MLSLLHTSTLAVLGALCVYGFP

SEQ ID NO: 9 - protein, 32 aa, Homo sapiens
PIKTDVITMDPKDNCSKDANDTLLLQLTNTSA

SEQ ID NO: 10 - protein, 23 aa, Homo sapiens
YYMYLLLLLKSVVYFAIITCCLL

SEQ ID NO: 11 - protein, 12 aa, Homo sapiens
RRTAFCCNGEKS

SEQ ID NO: 12 - protein, 21 aa, synthetic construct
  REGION 1..21: note = T2A self-cleaving sequence
GSGEGRGSLLTCGDVEENPGP

SEQ ID NO: 13 - protein, 20 aa, synthetic construct
  REGION 1..20: note = Signal Peptide (Light chain IgG leader sequence)
MSVPTQVLGLLLLWLTDARC

SEQ ID NO: 14 - protein, 48 aa, Homo sapiens
EVKTDSTDHVKPKETENTKQPSKSCHKPKAIVHTEKVNMMSLTVLGLR

SEQ ID NO: 15 - protein, 20 aa, Homo sapiens
MLFAKTVAVNFLLTAKLFFL

SEQ ID NO: 16 - protein, 89 aa, synthetic construct
  REGION 1..89: note = Precursor N-terminally truncated TCR Gamma 9 subunit
MLSLLHTSTLAVLGALCVYGFPPIKTDVITMDPKDNCSKDANDTLLLQLTNTSAYYMYLLLLLKSVVYFAIITCCLLRRTAFCCNGEKS

SEQ ID NO: 17 - protein, 67 aa, synthetic construct
  REGION 1..67: note = Mature N-terminally truncated TCR Gamma 9 subunit
PIKTDVITMDPKDNCSKDANDTLLLQLTNTSAYYMYLLLLLKSVVYFAIITCCLLRRTAFCCNGEKS

SEQ ID NO: 18 - protein, 331 aa, synthetic construct
  REGION 1..331: note = Precursor Chimeric anti-PD-L1 scFv-TCR-delta subunit
MSVPTQVLGLLLLWLTDARCEVQLVESGAEVKKPGSSVKVSCKASGGTFSSYAISWVRQAPGQGLEWMGGIIPIFGTANYAQKFQGRVTITADESTSTAYMELSSLRSEDTAVYYCARAPYYYYYMDVWGQGTTVTVSSGGGGSGGGGSGGGGSQSALTQPASVSGSLGQSVTISCTGSSSDVGSYNLVSWYQQHPGKAPNLMIYDVSKRSGVSNRFSGSKSGNTASLTISGLQAEDEADYYCSSYTGISTVVFGGGTKLTVLEVKTDSTDHVKPKETENTKQPSKSCHKPKAIVHTEKVNMMSLTVLGLRMLFAKTVAVNFLLTAKLFFL

SEQ ID NO: 19 - protein, 311 aa, synthetic construct
  REGION 1..311: note = Mature Chimeric anti-PD-L1 scFv-TCR-delta subunit
EVQLVESGAEVKKPGSSVKVSCKASGGTFSSYAISWVRQAPGQGLEWMGGIIPIFGTANYAQKFQGRVTITADESTSTAYMELSSLRSEDTAVYYCARAPYYYYYMDVWGQGTTVTVSSGGGGSGGGGSGGGGSQSALTQPASVSGSLGQSVTISCTGSSSDVGSYNLVSWYQQHPGKAPNLMIYDVSKRSGVSNRFSGSKSGNTASLTISGLQAEDEADYYCSSYTGISTVVFGGGTKLTVLEVKTDSTDHVKPKETENTKQPSKSCHKPKAIVHTEKVNMMSLTVLGLRMLFAKTVAVNFLLTAKLFFL

SEQ ID NO: 20 - DNA, 1323 bp, synthetic construct
  misc_feature 1..1323: note = Construct that includes sequence encoding Signal peptide - Chimeric anti-PD-L1/ gamma9 polypeptide - T2A sequence - Signal peptide - Truncated delta2 polypeptide
  source 1..1323: mol_type = other DNA
atggagtggtcctgggtgttcctgttctttctgtccgtgaccaccggtgtccactctgaagtccaactggtggaatctggagccgaggtaaagaaacctggctcaagtgtaaaagtatcatgcaaagcgtccggcggcacgttttcaagttacgccataagttgggtgagacaagctcccggacagggacttgagtggatgggaggaattatacctatttttgggactgcgaattatgcacaaaagttccagggaagggttactatcacagcggacgagagtacaagtacagcctatatggagttgtctagtctcagatctgaggacactgctgtctattattgtgcgcgagcgccttactattactactatatggacgtatggggccaaggcactacggtcacagtaagttctggcggcggcggcagcggtggcggtggctcaggtggtggtggttctcagagtgcccttactcagcctgccagtgtatccggttcacttggacaatcagttacaatatcttgtacgggctcttcatccgacgtgggcagttataatctcgtttcttggtatcaacaacaccccggcaaagcacccaatctgatgatctacgatgtcagtaaacgaagcggtgtcagtaacaggttctcaggttcaaaatccggcaataccgcgagtcttacaatttccggtttgcaggcggaggatgaggccgactactattgttcatcctatacaggtattagcaccgttgtgtttggcgggggcactaaactcactgttctccccatcaagaccgatgtgatcacaatggaccccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagcggctctggagctactaacttcagcctgctgaagcaggctggagacgtggaggagaaccctggacctatgcagaggatttccagtctcattcacctcagcttgttttgggcaggcgtcatgtctactgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctg

SEQ ID NO: 21 - protein, 440 aa, synthetic construct
  REGION 1..440: note = Precursor PD-L1 scFv-gamma-delta-TCR polypeptides encoded by SEQ ID NO:20
MEWSWVFLFFLSVTTGVHSEVQLVESGAEVKKPGSSVKVSCKASGGTFSSYAISWVRQAPGQGLEWMGGIIPIFGTANYAQKFQGRVTITADESTSTAYMELSSLRSEDTAVYYCARAPYYYYYMDVWGQGTTVTVSSGGGGSGGGGSGGGGSQSALTQPASVSGSLGQSVTISCTGSSSDVGSYNLVSWYQQHPGKAPNLMIYDVSKRSGVSNRFSGSKSGNTASLTISGLQAEDEADYYCSSYTGISTVVFGGGTKLTVLPIKTDVITMDPKDNCSKDANDTLLLQLTNTSAYYMYLLLLLKSVVYFAIITCCLLRRTAFCCNGEKSGSGEGRGSLLTCGDVEENPGPMQRISSLIHLSLFWAGVMSTDFEVKTDSTDHVKPKETENTKQPSKSCHKPKAIVHTEKVNMMSLTVLGLRMLFAKTVAVNFLLTAKLFFL

SEQ ID NO: 22 - protein, 19 aa, Mus musculus
MEWSWVFLFFLSVTTGVHS

SEQ ID NO: 23 - protein, 22 aa, synthetic construct
  REGION 1..22: note = Signal peptide
MQRISSLIHLSLFWAGVMSTDF

SEQ ID NO: 24 - protein, 329 aa, synthetic construct
  REGION 1..329: note = Precursor Chimeric anti-PD-L1 scFv-TCR-gamma subunit
MEWSWVFLFFLSVTTGVHSEVQLVESGAEVKKPGSSVKVSCKASGGTFSSYAISWVRQAPGQGLEWMGGIIPIFGTANYAQKFQGRVTITADESTSTAYMELSSLRSEDTAVYYCARAPYYYYYMDVWGQGTTVTVSSGGGGSGGGGSGGGGSQSALTQPASVSGSLGQSVTISCTGSSSDVGSYNLVSWYQQHPGKAPNLMIYDVSKRSGVSNRFSGSKSGNTASLTISGLQAEDEADYYCSSYTGISTVVFGGGTKLTVLPIKTDVITMDPKDNCSKDANDTLLLQLTNTSAYYMYLLLLLKSVVYFAIITCCLLRRTAFCCNGEKS

SEQ ID NO: 25 - protein, 310 aa, synthetic construct
  REGION 1..310: note = Mature Chimeric anti-PD-L1 scFv-TCR-gamma subunit
EVQLVESGAEVKKPGSSVKVSCKASGGTFSSYAISWVRQAPGQGLEWMGGIIPIFGTANYAQKFQGRVTITADESTSTAYMELSSLRSEDTAVYYCARAPYYYYYMDVWGQGTTVTVSSGGGGSGGGGSGGGGSQSALTQPASVSGSLGQSVTISCTGSSSDVGSYNLVSWYQQHPGKAPNLMIYDVSKRSGVSNRFSGSKSGNTASLTISGLQAEDEADYYCSSYTGISTVVFGGGTKLTVLPIKTDVITMDPKDNCSKDANDTLLLQLTNTSAYYMYLLLLLKSVVYFAIITCCLLRRTAFCCNGEKS

SEQ ID NO: 26 - protein, 90 aa, synthetic construct
  REGION 1..90: note = Precursor N-terminally truncated TCR-delta subunit
MQRISSLIHLSLFWAGVMSTDFEVKTDSTDHVKPKETENTKQPSKSCHKPKAIVHTEKVNMMSLTVLGLRMLFAKTVAVNFLLTAKLFFL

SEQ ID NO: 27 - protein, 68 aa, synthetic construct
  REGION 1..68: note = Mature N-terminally truncated TCR-delta subunit
EVKTDSTDHVKPKETENTKQPSKSCHKPKAIVHTEKVNMMSLTVLGLRMLFAKTVAVNFLLTAKLFFL

SEQ ID NO: 28 - DNA, 1593 bp, synthetic construct
  misc_feature 1..1593: note = PD-L1 CAR construct
  source 1..1593: mol_type = other DNA
atggagtggtcctgggtgttcctgttctttctgtccgtgaccaccggtgtccactctgaagtccaactggtggaatctggagccgaggtaaagaaacctggctcaagtgtaaaagtatcatgcaaagcgtccggcggcacgttttcaagttacgccataagttgggtgagacaagctcccggacagggacttgagtggatgggaggaattatacctatttttgggactgcgaattatgcacaaaagttccagggaagggttactatcacagcggacgagagtacaagtacagcctatatggagttgtctagtctcagatctgaggacactgctgtctattattgtgcgcgagcgccttactattactactatatggacgtatggggccaaggcactacggtcacagtaagttctggcggcggcggcagcggtggcggtggctcaggtggtggtggttctcagagtgcccttactcagcctgccagtgtatccggttcacttggacaatcagttacaatatcttgtacgggctcttcatccgacgtgggcagttataatctcgtttcttggtatcaacaacaccccggcaaagcacccaatctgatgatctacgatgtcagtaaacgaagcggtgtcagtaacaggttctcaggttcaaaatccggcaataccgcgagtcttacaatttccggtttgcaggcggaggatgaggccgactactattgttcatcctatacaggtattagcaccgttgtgtttggcgggggcactaaactcactgttctcgctaagccgaccacgacaccggctccaagacctccgacgccagctccaacgatagcgtcacagccattgtctctccgccctgaagcctgccggcccgctgcgggcggcgcggttcatacccggggattggactttgcccccagaaagatagaggtgatgtaccctcccccctacttggacaacgaaaagtctaatggcactatcattcacgtaaagggcaaacacctttgtccaagtcctttgttcccaggcccatctaagccgttctgggtactcgtggttgtggggggcgtgctcgcttgttactcactgctggtgacggtggcctttattattttctgggttaaacggggcagaaagaaactcctgtatatattcaaacaaccatttatgagaccagtacaaactactcaagaggaagatggctgtagctgccgatttccagaagaagaagaaggaggatgtgaactgagggtaaaatttagcaggtctgcagatgcgcctgcgtatcaacagggtcagaatcagctctataatgagctgaacctcgggcggcgggaagagtatgatgttctcgataaaaggagaggacgagaccccgaaatgggcggcaaaccgagacgcaaaaatcctcaggaggggctctacaatgaacttcaaaaagacaaaatggccgaagcatactcagaaatcggaatgaaaggggagaggagacgcgggaagggccatgatggactgtatcagggactttccacagccaccaaggacacctatgacgctctccacatgcaggccctgccccctcgc

SEQ ID NO: 29 - protein, 531 aa, synthetic construct
  REGION 1..531: note = PD-L1 CAR (Precursor)
MEWSWVFLFFLSVTTGVHSEVQLVESGAEVKKPGSSVKVSCKASGGTFSSYAISWVRQAPGQGLEWMGGIIPIFGTANYAQKFQGRVTITADESTSTAYMELSSLRSEDTAVYYCARAPYYYYYMDVWGQGTTVTVSSGGGGSGGGGSGGGGSQSALTQPASVSGSLGQSVTISCTGSSSDVGSYNLVSWYQQHPGKAPNLMIYDVSKRSGVSNRFSGSKSGNTASLTISGLQAEDEADYYCSSYTGISTVVFGGGTKLTVLAKPTTTPAPRPPTPAPTIASQPLSLRPEACRPAAGGAVHTRGLDFAPRKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 46 aa, Homo sapiens
AKPTTTPAPRPPTPAPTIASQPLSLRPEACRPAAGGAVHTRGLDFA

SEQ ID NO: 31 - protein, 42 aa, Homo sapiens
PRKIEVMYPPPYLDNEKSNGTIIHVKGKHLCPSPLFPGPSKP

SEQ ID NO: 32 - protein, 27 aa, Homo sapiens
FWVLVVVGGVLACYSLLVTVAFIIFWV

SEQ ID NO: 33 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 42 aa, Homo sapiens
KRGRKKLLYIFKQPFMRPVQTTQEEDGCSCRFPEEEEGGCEL

SEQ ID NO: 35 - DNA, 195 bp, synthetic construct
  misc_feature 1..195: note = JeT promoter
  source 1..195: mol_type = other DNA
gaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcagttgaca

SEQ ID NO: 36 - DNA, 603 bp, unidentified
  source 1..603: mol_type = genomic DNA; note = Cytomegalovirus
aagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgactctactaga

SEQ ID NO: 37 - DNA, 660 bp, Homo sapiens
  source 1..660: mol_type = genomic DNA
ggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccaca

SEQ ID NO: 38 - DNA, 650 bp, Homo sapiens
  source 1..650: mol_type = genomic DNA
gatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatcttt

SEQ ID NO: 39 - DNA, 19 bp, Homo sapiens
  source 1..19: mol_type = genomic DNA
cagggttctggatatctgt

SEQ ID NO: 40 - DNA, 20 bp, synthetic construct
  misc_feature 1..20: note = Forward primer
  misc_feature 1..2: note = phosphorothioate linkage
  misc_feature 3: note = n = 2'-O-methylcytidine
  misc_feature 3..4: note = phosphorothioate linkage
  misc_feature 4: note = n = 2'-O-methyladenosine
  misc_feature 4..5: note = phosphorothioate linkage
  misc_feature 5: note = n = 2'-O-methylcytidine
  source 1..20: mol_type = other DNA
atnnngagcagctggtttct

SEQ ID NO: 41 - DNA, 24 bp, synthetic construct
  misc_feature 1..24: note = Reverse primer
  source 1..24: mol_type = other DNA
gacctcatgtctagcacagttttg

SEQ ID NO: 42 - DNA, 171 bp, Homo sapiens
  source 1..171: mol_type = genomic DNA
atcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccaca

SEQ ID NO: 43 - DNA, 161 bp, Homo sapiens
  source 1..161: mol_type = genomic DNA
gatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtc

SEQ ID NO: 44 - protein, 17 aa, synthetic construct
  REGION 1..17: note = Alternative GS linker
GGGSGGGSGGGSGGGSG

SEQ ID NO: 45 - protein, 100 aa, synthetic construct
  REGION 1..100: note = Alternative linker
  REGION 6..100: note = MISC_FEATURE - May be present or absent; (GGGSE)n where n is 1 to 20
GGGSEGGGSEGGGSEGGGSEGGGSEGGGSEGGGSEGGGSEGGGSEGGGSEGGGSEGGGSEGGGSEGGGSEGGGSEGGGSEGGGSEGGGSEGGGSEGGGSE

SEQ ID NO: 46 - protein, 5 aa, synthetic construct
  REGION 1..5: note = Anti-PD-L1 Heavy Chain CDR1
SYAIS

SEQ ID NO: 47 - protein, 17 aa, synthetic construct
  REGION 1..17: note = Anti-PD-L1 Heavy Chain CDR2
GIIPIFGTANYAQKFQG

SEQ ID NO: 48 - protein, 10 aa, synthetic construct
  REGION 1..10: note = Anti-PD-L1 Heavy Chain CDR3
APYYYYYMDV

SEQ ID NO: 49 - protein, 14 aa, synthetic construct
  REGION 1..14: note = Anti-PD-L1 Light Chain CDR1
TGSSSDVGSYNLVS

SEQ ID NO: 50 - protein, 6 aa, synthetic construct
  REGION 1..6: note = Anti-PD-L1 Light Chain CDR2
DVSKRS

SEQ ID NO: 51 - protein, 10 aa, synthetic construct
  REGION 1..10: note = Anti-PD-L1 Light Chain CDR3
SSYTGISTVV

SEQ ID NO: 52 - protein, 22 aa, synthetic construct
  REGION 1..22: note = P2A self-cleaving sequence
GSGATNFSLLKQAGDVEENPGP

SEQ ID NO: 53 - protein, 23 aa, synthetic construct
  REGION 1..23: note = E2A self-cleaving sequence
GSGQCTNYALLKLAGDVESNPGP

SEQ ID NO: 54 - protein, 25 aa, synthetic construct
  REGION 1..25: note = F2A self-cleaving sequence
GSGVKQTLNFDLLKLAGDVESNPGP

SEQ ID NO: 55 - protein, 21 aa, Homo sapiens
MALPVTALLLPLALLLHAARP

SEQ ID NO: 56 - protein, 20 aa, synthetic construct
  REGION 1..20: note = Signal Peptide
MEFGLSWVFLVALFRGVQCD

SEQ ID NO: 57 - protein, 16 aa, synthetic construct
  REGION 1..16: note = Signal Peptide
METDTLLLWVLLLWVP

SEQ ID NO: 58 - protein, 120 aa, synthetic construct
  REGION 1..120: note = Heavy chain variable region anti-CD19 FMC63 antibody
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 59 - protein, 107 aa, synthetic construct
  REGION 1..107: note = Light chain variable region anti-CD19 FMC63 antibody
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 60 - protein, 242 aa, synthetic construct
  REGION 1..242: note = anti-CD19 ScFv
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 61 - protein, 23 aa, Homo sapiens
IYIWAPLAGTCGVLLLSLVITLY

SEQ ID NO: 62 - protein, 309 aa, synthetic construct
  REGION 1..309: note = Mature Chimeric anti-CD19 scFv-TCR-gamma subunit
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITPIKTDVITMDPKDNCSKDANDTLLLQLTNTSAYYMYLLLLLKSVVYFAIITCCLLRRTAFCCNGEKS

SEQ ID NO: 63 - protein, 328 aa, synthetic construct
  REGION 1..328: note = Precursor Chimeric anti-CD19 scFv-TCR-gamma subunit
MEWSWVFLFFLSVTTGVHSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITPIKTDVITMDPKDNCSKDANDTLLLQLTNTSAYYMYLLLLLKSVVYFAIITCCLLRRTAFCCNGEKS

SEQ ID NO: 64 - DNA, 987 bp, synthetic construct
  misc_feature 1..987: note = Encodes precursor Chimeric anti-CD19 scFv-TCR-gamma subunit
  source 1..987: mol_type = other DNA
atgtccgtccctacccaggtgctgggcctgctgctgctgtggctgaccgatgctagatgcgatatccagatgacacagaccaccagcagcctgagcgccagcctgggcgaccgagtgactatcagctgccgggcatcccaggatatttctaagtatctgaactggtaccagcagaagcccgacggcactgtcaaactgctgatctaccacaccagtagactgcattcaggggtgcctagcaggttctccggatctggcagtgggactgactactccctgaccatctctaacctggagcaggaagatattgccacctatttctgccagcagggcaatacactgccttacacttttggcgggggaacaaagctggagatcactggcggcggcggcagcggtggcggtggctcaggtggtggtggttctgaggtgaaactgcaggaaagcggaccaggactggtcgcaccttcacagagcctgtccgtgacatgtactgtctccggagtgtctctgcccgattacggcgtctcttggatccggcagccccctagaaagggactggagtggctgggcgtgatctggggaagtgaaactacctactataatagtgctctgaaatcaagactgaccatcattaaggacaactctaaaagtcaggtgtttctgaagatgaattccctgcagaccgacgatacagcaatctactattgcgccaaacactactattacggcgggagctatgccatggattactgggggcagggaacttccgtcaccgtgagcagccccatcaagaccgatgtgatcacaatggaccccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagc

SEQ ID NO: 65 - DNA, 927 bp, synthetic construct
  misc_feature 1..927: note = Encodes mature Chimeric anti-CD19 scFv-TCR-gamma subunit (without signal peptide)
  source 1..927: mol_type = other DNA
gatatccagatgacacagaccaccagcagcctgagcgccagcctgggcgaccgagtgactatcagctgccgggcatcccaggatatttctaagtatctgaactggtaccagcagaagcccgacggcactgtcaaactgctgatctaccacaccagtagactgcattcaggggtgcctagcaggttctccggatctggcagtgggactgactactccctgaccatctctaacctggagcaggaagatattgccacctatttctgccagcagggcaatacactgccttacacttttggcgggggaacaaagctggagatcactggcggcggcggcagcggtggcggtggctcaggtggtggtggttctgaggtgaaactgcaggaaagcggaccaggactggtcgcaccttcacagagcctgtccgtgacatgtactgtctccggagtgtctctgcccgattacggcgtctcttggatccggcagccccctagaaagggactggagtggctgggcgtgatctggggaagtgaaactacctactataatagtgctctgaaatcaagactgaccatcattaaggacaactctaaaagtcaggtgtttctgaagatgaattccctgcagaccgacgatacagcaatctactattgcgccaaacactactattacggcgggagctatgccatggattactgggggcagggaacttccgtcaccgtgagcagccccatcaagaccgatgtgatcacaatggaccccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagc